(19) United States
(12) Patent Application Publication
(10) Pub. No.: US 2011/0172297 A1
(43) Pub. Date: Jul. 14, 2011

(54) Oxopiperdinyl And Pyranyl Sulfonamides and Pharmaceutical Compositions Thereof

(75) Inventors: Kimberly Gail Estep, Groton, CT (US); Antone F.J. Fliri, Stonington, CT (US); Randall James Gallaschun, Lebanon, CT (US); Christopher John O'Donnell, Mystic, CT (US); Nandini Chaturbhai Patel, Providence, RI (US); Jacob Bradley Schwarz, Waterford, CT (US); Longfei Xie, Groton, CT (US)

(73) Assignee: Pfizer Inc

(21) Appl. No.: 13/061,684

(22) PCT Filed: Sep. 18, 2009
(86) PCT No.: PCT/IB09/54109

§ 371 Date: Mar. 29, 2011

Related U.S. Application Data
(60) Provisional application No. 61/102,095, filed on Oct. 2, 2008.

(51) Int. Cl. A61K 31/351; C07D 309/14; C07D 409/10; C07D 405/10; C07D 211/56; A61K 31/381; A61P 25/00; A61P 25/28; A61P 9/10; A61P 27/02; A61P 25/16; A61P 25/08; A61P 25/06; A61P 25/18; A61P 25/22

(52) U.S. Cl. 514/444; 549/424; 549/419; 549/60; 546/282.1; 546/216; 546/213; 514/459

(57) ABSTRACT

The invention is directed to a class of compounds, including the pharmaceutically acceptable salts of the compounds, having the structure of Formula (I): as defined in the specification. The invention is also directed to compositions containing the compounds of Formula (I) as AMPA modulators.

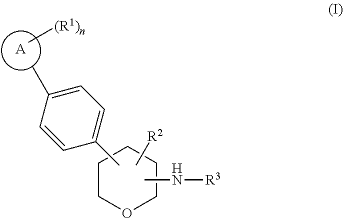

### FIELD OF THE INVENTION

The present invention relates to a novel class of compounds having the structure of Formula I or II as defined herein and pharmaceutical compositions comprising a compound of Formula I or II. The present invention also comprises methods of treating a subject by administering a therapeutically effective amount of a compound of Formula I or II to the subject. These compounds are useful for the conditions disclosed herein. The present invention further comprises methods for making the compounds of Formula I or II and corresponding intermediates.

### BACKGROUND OF THE INVENTION

The present invention provides compounds of Formula I or II, pharmaceutical compositions thereof, and methods of using the same, processes for preparing the same, and intermediates thereof.

The primary excitatory neurotransmitter in the mammalian central nervous system (CNS) is the amino acid glutamate whose signal transduction is mediated by either ionotropic or metabotropic glutamate receptors (GluR). Ionotropic glutamate receptors (iGluR) are comprised of three subtypes differentiated by their unique responses to the three selective iGluR agonists α-amino-3-hydroxy-5-methylisoxazole-4-propionic acid (AMPA), N-methyl-D-aspartate (NMDA) and kainate (Parsons, C. G., Danysz, W. and Lodge, D. (2002), in: Ionotropic Glutamate Receptors as Therapeutic Targets (Danysz, W., Lodge, D. and Parsons, C. G. eds), pp 1-30, F.P. Graham Publishing Co., Tennessee). AMPA receptors, proteinaceous homo- or heterotetramers comprised of any combination of four ca. 900 amino acid monomer subunits each encoded from a distinct gene (Glu_{A1-A4}) with each subunit protein existing as one of two splice variants deemed “flip” and “flop”, mediate the vast majority of excitatory synaptic transmissions in the mammalian brain and have long been proposed to be an integral component of the neural circuitry that mediates cognitive processes (Bleakman, D. and Lodge, D. (1998) Neuropharmacology of AMPA and Kainate Receptors. Neuropharmacology 37:1187-1204). The combination of various heterotetrameric possibilities, two splice forms for each of the four iGluR monomers and receptor subunit RNA editing with the heterogeneous distribution of AMPA receptors throughout the brain highlight the myriad of potential AMPA receptor responses within this organ (Black, M. D. (2005) Therapeutic Potential of Positive AMPA Modulators and Their Relationship to AMPA Receptor Subunits. A Review of Preclinical Data. Psychopharmacology 179:154-163). AMPA modulators have now become an active target for drug discovery (see Rogers, B. and Schmidt, C., (2006) Novel Approaches for the Treatment of Schizophrenia, Annual Reports in Medicinal Chemistry 3-21).

### SUMMARY OF THE INVENTION

The present invention is directed to a class of compounds, including the pharmaceutically acceptable salts of the compounds, having the structure of Formula I:

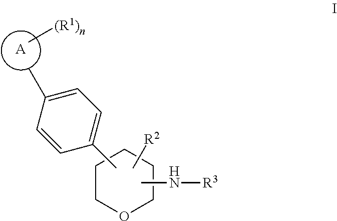

wherein R¹ is hydrogen, fluoro, (C₁-C₂)alkyl optionally substituted with one to five fluoro, (C₁-C₂)alkoxy optionally substituted with one to five fluoro, —(C═O)—NH₂, —(C═O)—NH—(C₁-C₂)alkyl, —(C═O)—N[(C₁-C₂)alkyl]₂ or —CN;

n is zero, one, or two;

R² is hydrogen or hydroxyl;

R³ is (C₁-C₅)alkyl-(C═O)—, [(C₁-C₃)alkyl]₂N—(C═O)—, (C₁-C₅)alkyl-SO₂—, (C₃-C₅)cycloalkyl-SO₂—, or [(C₁-C₃)alkyl]₂N—SO₂—; wherein said (C₁-C₃)alkyl moieties of said [(C₁-C₃)alkyl]₂N—(C═O)— and [(C₁-C₃)alkyl]₂N—SO₂— may optionally be taken together with the nitrogen atom to which they are attached to form a four to six membered heterocyclic ring;

and ring “A” is phenyl, pyridyl, or thienyl.

The term “alkyl” refers to a linear or branched-chain saturated hydrocarbyl substituent (i.e., a substituent obtained from a hydrocarbon by removal of a hydrogen) containing from one to three or one to two carbon atoms. Examples of such substituents include methyl, ethyl, propyl (including n-propyl and isopropyl), and the like.

In some instances, the number of carbon atoms in a hydrocarbyl substituent (e.g., alkyl, alkenyl, cycloalkyl, cycloalkenyl, aryl, etc.) is indicated by the prefix “Cₓ-C_{y}—,” wherein x is the minimum and y is the maximum number of carbon atoms in the substituent. Thus, for example, “C₁-C₃-alkyl” refers to an alkyl substituent containing from 1 to 3 carbon atoms.

The term “hydrogen” refers to a hydrogen substituent, and may be depicted as —H.

The term “hydroxy” or “hydroxyl” refers to —OH. When used in combination with another term(s), the prefix “hydroxy” indicates that the substituent to which the prefix is attached is substituted with one or more hydroxy substituents. Compounds bearing a carbon to which one or more hydroxy substituents are attached include, for example, alcohols, enols and phenol.

The term “cyano” (also referred to as “nitrile”) means —CN, which also may be depicted:

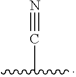

The term “carbonyl” means —(C═O)—, or >C═O which also may be depicted as:

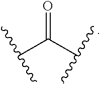

The term “amino” refers to —NH₂.

The term “oxo” refers to ═O.

The term “amido” refers to RN—(C═O)—, where R is hydrogen or alkyl. Examples of amides and alkyl amides include dimethyl and diethyl amides.

The term “alkoxy” refers to an alkyl linked to an oxygen, which may also be represented as:

—O—R, wherein the R represents the alkyl group. Examples of alkoxy include methoxy, and ethoxy.

The term “sulfonyl” refers to —SO₂, which also may be depicted as:

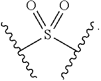

Thus, for example, “alkyl-sulfonyl-alkyl” refers to alkyl-SO₂-alkyl. Examples of alkylsulfonyl include methylsulfonyl, ethylsulfonyl, and propylsulfonyl.

The term “(alkyl)₂N—SO₂—” refers to sulfonamides such as dimethyl or diethyl sulfonamides.

If substituents are described as being “independently selected” from a group, each substituent is selected independent of the other. Each substituent therefore may be identical to or different from the other substituent(s).

As used herein the terms “Formula I” or “Formula II,” (Formula II is described herein below and these terms are hereinafter understood to also include Formula I, Ia, Ib, Ic, Id, Ie, If, II, IIa, IIb or IIc and additionally are understood to include pharmaceutically acceptable salts thereof), are hereinafter referred to as a “compound(s) of the invention.” Such terms are also defined to include all forms of the compound of Formula I or II, including hydrates, solvates, isomers, crystalline and non-crystalline forms, isomorphs, polymorphs, and metabolites thereof.

Compounds of Formula I or II may exist in unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of Formula I or II may have asymmetric carbon atoms. The carbon-carbon bonds of the compounds of Formula I or II may be depicted herein using a solid line [symbol] a solid wedge [symbol] or a dotted wedge [symbol] The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of Formula I or II may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of Formula I or II can exist as enantiomers and diastereomers or as racemates and mixtures thereof. The use of a solid line to depict bonds to one or more asymmetric carbon atoms in a compound of Formula I or II and the use of a solid or dotted wedge to depict bonds to other asymmetric carbon atoms in the same compound is meant to indicate that a mixture of diastereomers is present.

Stereoisomers of Formula I or II include cis and trans isomers, optical isomers such as R and S enantiomers, diastereomers, geometric isomers, rotational isomers, conformational isomers, and tautomers of the compounds of Formula I or II, including compounds exhibiting more than one type of isomerism; and mixtures thereof (such as racemates and diastereomeric pairs). Also included are acid addition or base addition salts wherein the counterion is optically active, for example, D-lactate or L-lysine, or racemic, for example, DL-tartrate or DL-arginine.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of Formula I or II with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclylic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sulfanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

In one embodiment, hemisalts of acids and bases may also be formed, for example, hemisulphate and hemicalcium salts.

The present invention also includes isotopically labelled compounds, which are identical to those recited in Formula I or II, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of Formula I or II of this invention and prodrugs thereof can generally be prepared by carrying out the procedures disclosed in the Schemes and/or in the Examples below, by substituting a readily available isotopically labelled reagent for a non-isotopically labelled reagent.

In one embodiment of the invention, the compound of Formula I has the stereochemistry depicted below for Formula Ia:

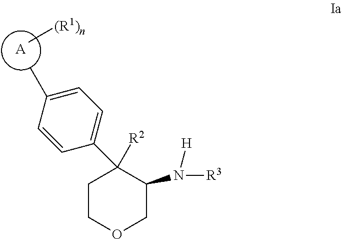

In another embodiment of the invention, the compound of formula I has the stereochemistry depicted below for Formula Ib:

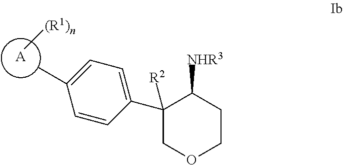

In yet another embodiment of the present invention the stereochemistry about the pyranyl ring is depicted below for Formula Ic:

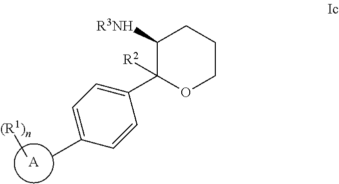

In yet another embodiment of the present invention the stereochemistry about the pyranyl ring is depicted below for Formula Id:

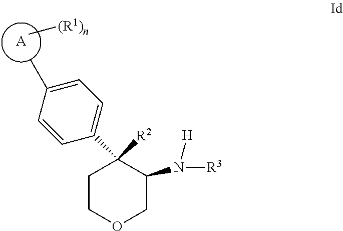

In yet another embodiment of the present invention the stereochemistry about the pyranyl ring is depicted below for Formula Ie:

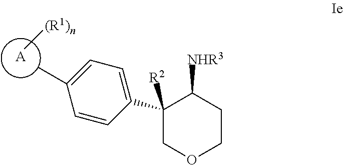

In one embodiment of the invention, the compound of Formula I has the stereochemistry depicted below for Formula If:

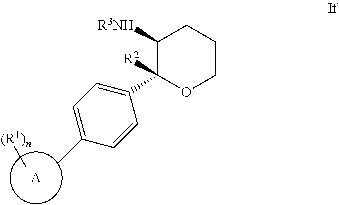

In one embodiment of the invention, the compound of Formula I has the stereochemistry depicted below for Formula Ig:

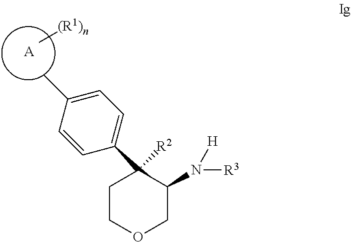

In one embodiment of the invention, the compound of Formula I has the stereochemistry depicted below for Formula Ih:

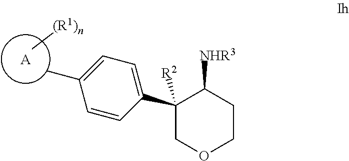

In one embodiment of the invention, the compound of Formula I has the stereochemistry depicted below for Formula Ii:

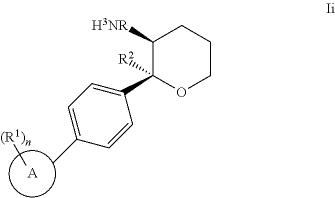

Yet other embodiments of the present invention relate to so called amido pyranes of Formula I (and Ia, Ib, Ic, Id, Ie, If, Ig, Ih and Ii) wherein R³ is (C₁-C₅)alkyl-(C═O)—.

Yet other embodiments of the present invention relate to so called ureopyranes of Formula I (and Ia, Ib, Ic, Id, Ie, If, Ig, Ih and Ii) wherein R³ is [(C₁-C₂)alkyl]₂N—(C═O)—, wherein said (C₁-C₂)alkyl moieties may optionally be taken together with the nitrogen atom to which they are attached to form a four to six membered heterocyclic ring.

Yet other embodiments of the present invention relate to alkylsulfonylpyranes of Formula I (and Ia, Ib, Ic, Id, Ie, If, Ig, Ih and Ii) wherein R³ is (C₁-C₅)alkyl-SO₂—.

Yet other embodiments of the present invention relate to cycloalkylsulonylpyranes of Formula I (and Ia, Ib, Ic, Id, Ie, If, Ig, Ih and Ii) wherein R³ is (C₃-C₅)cycloalkyl-SO₂—.

Yet other embodiments of the present invention relate to sulfonamidopyranes of Formula I (and Ia, Ib, Ic, Id, Ie, If, Ig, Ih and Ii) wherein R³ is [(C₁-C₂)alkyl]₂N—SO₂—; wherein said (C₁-C₂)alkyl moieties may optionally be taken together with the nitrogen atom to which they are attached to form a four to six membered heterocyclic ring.

Yet other embodiments of interest to the present inventors include compounds of Formula I (and Ia, Ib, Ic, Id, Ie, If, Ig, Ih and Ii) including the R³ embodiments described above wherein R² is hydrogen.

Yet other embodiments of interest to the present inventors include compounds of Formula I (and Ia, Ib, Ic, Id, Ie, If, Ig, Ih and Ii) including the R³ embodiments described above wherein R² is hydroxy.

Yet other embodiments of interest to the present inventors include compounds of Formula I (and Ia, Ib, Ic, Id, Ie, If, Ig, Ih and Ii) including the R³ embodiments described above and the R² hydrogen or hydroxyl embodiments also described above, wherein n is zero or one.

Yet other embodiments of interest to the present inventors include compounds of Formula I (and Ia, Ib, Ic, Id, Ie, If, Ig, Ih and Ii) including the R³ embodiments described above and the R² hydrogen or hydroxyl embodiments also described above, wherein n is two.

Yet other embodiments of interest to the present inventors include compounds of Formula I (and Ia, Ib, Ic, Id, Ie, If, Ig, Ih and Ii) including the R³ embodiments described above and the R² hydrogen or hydroxyl embodiments described above, and the “n” zero or one and “n” is two embodiments also described above, also including the embodiments comprising combinations of each of the foregoing embodiment groups (e.g. Formula Ia with the R³ sulfonamidopyranes and R² hydrogen and “n” two embodiments); wherein one R¹ is fluoro, (C₁-C₂)alkoxy optionally substituted with one to five fluoro (more specifically ethoxy) or —CN (even more specifically wherein this R¹ is an ortho substituent).

Yet other embodiments of interest to the present inventors include compounds of Formula I (and Ia, Ib, Ic, Id, Ie, If, Ig, Ih and Ii) including the R³ embodiments described above and the R² hydrogen or hydroxyl embodiments described above, and the “n” is zero or one and “n” is two embodiments also described above, also including the embodiments comprising combinations of each of the foregoing embodiment groups (e.g. Formula Ia with the R³ sulfonamidopyranes and R² hydrogen and “n” is two embodiments); wherein one R¹ is (C₁-C₂)alkyl optionally substituted with one to five fluoro.

Exemplary compounds according to the invention include the specific compounds disclosed herein or pharmaceutically acceptable salts thereof.

Another embodiment of interest to the present inventors includes a group of (3R,4R)-4-biaryl-pyranes selected from the group:
N-[(3R,4R)-4-biphenyl-4-yltetrahydro-2H-pyran-3-yl]propane-2-sulfonamide;
N-[(3R,4R)-4-(2′-ethoxybiphenyl-4-yl)tetrahydro-2H-pyran-3-yl]propane-2-sulfonamide;
N-[(3R,4R)-4-(2′-cyanobiphenyl-4-yl)tetrahydro-2H-pyran-3-yl]propane-2-sulfonamide; and
N-{(3R,4R)-4-[4-(5-cyano-2-thienyl)phenyl]tetrahydro-2H-pyran-3-yl}propane-2-sulfonamide, or the pharmaceutically acceptable salts thereof.

Another embodiment of interest to the present inventors includes the compound N-[(3R,4R)-3-(2′-cyanobiphenyl-4-yl)tetrahydro-2H-pyran-4-yl]propane-2-sulfonamide, or the pharmaceutically acceptable salts thereof.

Another embodiment of interest to the present inventors includes a group of (3S,4S)-3-biaryl-pyranes selected from the group:
N-{(3S,4S)-3-[4-(5-cyano-2-thienyl)phenyl]tetrahydro-2H-pyran-4-yl}propane-2-sulfonamide;
N-[(3S,4S)-3-(2′-cyanobiphenyl-4-yl)tetrahydro-2H-pyran-4-yl]propane-2-sulfonamide;
N-[(3S,4S)-3-(2′-cyano-4′-fluorobiphenyl-4-yl)tetrahydro-2H-pyran-4-yl]propane-2-sulfonamide;
N-[(3S,4S)-3-biphenyl-4-yltetrahydro-2H-pyran-4-yl]propane-2-sulfonamide;
N-[(3S,4S)-3-(2′-ethoxybiphenyl-4-yl)tetrahydro-2H-pyran-4-yl]propane-2-sulfonamide; and
N-[(3S,4S)-3-(4′-cyanobiphenyl-4-yl)tetrahydro-2H-pyran-4-yl]propane-2-sulfonamide, or the pharmaceutically acceptable salts thereof.

Another embodiment of interest to the present inventors includes a group of (3R,4S)-3-biaryl-pyranes selected from the group:
N-[(3R,4S)-3-biphenyl-4-yltetrahydro-2H-pyran-4-yl]propane-2-sulfonamide;
N-[(3R,4S)-3-(2′-cyanobiphenyl-4-yl)tetrahydro-2H-pyran-4-yl]propane-2-sulfonamide;
N-[(3R,4S)-3-(2′-ethoxybiphenyl-4-yl)tetrahydro-2H-pyran-4-yl]propane-2-sulfonamide;
N-[(3R,4S)-3-(4′-cyanobiphenyl-4-yl)tetrahydro-2H-pyran-4-yl]propane-2-sulfonamide;
N-[(3R,4S)-3-(2′-cyano-4′-fluorobiphenyl-4-yl)tetrahydro-2H-pyran-4-yl]propane-2-sulfonamide;
N-{(3R,4S)-3-[4-(5-cyano-2-thienyl)phenyl]tetrahydro-2H-pyran-4-yl}propane-2-sulfonamide; or the pharmaceutically acceptable salts thereof.

Another embodiment of interest to the present inventors includes a group of (3R,4S)-4-biaryl pyranes selected from the group:
N-[(3R,4S)-4-(2′-cyanobiphenyl-4-yl)tetrahydro-2H-pyran-3-yl]propane-2-sulfonamide;
N-{(3R,4S)-4-[4-(5-cyano-2-thienyl)phenyl]tetrahydro-2H-pyran-3-yl}propane-2-sulfonamide; and
N-[(3R,4S)-4-(2′-ethoxybiphenyl-4-yl)tetrahydro-2H-pyran-3-yl]propane-2-sulfonamide, or the pharmaceutically acceptable salts thereof.

Another embodiment of interest to the present inventors includes a group of (3S,4R)-3-biaryl-pyranes selected from the group:
N-[(3S,4R)-3-(2′-ethoxybiphenyl-4-yl)tetrahydro-2H-pyran-4-yl]propane-2-sulfonamide; and
N-[(3S,4R)-3-(4-pyridin-3-ylphenyl)tetrahydro-2H-pyran-4-yl]propane-2-sulfonamide; or the pharmaceutically acceptable salts thereof.

Another embodiment of interest to the inventors includes (2S,3R)-2-biaryl-pyranes including:
N-[(2S,3R)-2-(2′-ethoxybiphenyl-4-yl)tetrahydro-2H-pyran-3-yl]propane-2-sulfonamide; and
N-[(2S,3R)-2-(2′-cyanobiphenyl-4-yl)tetrahydro-2H-pyran-3-yl]propane-2-sulfonamide; or the pharmaceutically acceptable salts thereof.

Another embodiment of interest to the inventors includes a group of (2R,3S)-2-biaryl-pyranes selected from the group:
N-[(2R,3S)-2-biphenyl-4-yltetrahydro-2H-pyran-3-yl]propane-2-sulfonamide;
N-[(2R,3S)-2-(2′-ethoxybiphenyl-4-yl)tetrahydro-2H-pyran-3-yl]propane-2-sulfonamide;
N-[(2R,3S)-2-(2′-cyanobiphenyl-4-yl)tetrahydro-2H-pyran-3-yl]propane-2-sulfonamide; and
N-{(2R,3S)-2-[4-(5-cyano-2-thienyl)phenyl]tetrahydro-2H-pyran-3-yl}propane-2-sulfonamide, or the pharmaceutically acceptable salts thereof.

Another embodiment of interest to the present inventors includes a group of 4-hydroxy-4-biaryl-pyranes selected from the group consisting of:
N-[(3S,4R)-4-biphenyl-4-yl-4-hydroxytetrahydro-2H-pyran-3-yl]propane-2-sulfonamide; and
N-[(3R,4S)-4-biphenyl-4-yl-4-hydroxytetrahydro-2H-pyran-3-yl]propane-2-sulfonamide; or the pharmaceutically acceptable salts thereof.

The present invention is also directed to a compound of the Formula

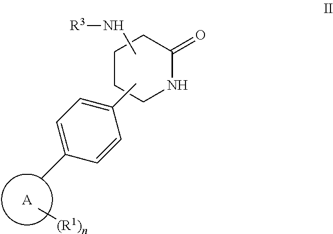

wherein R¹ is hydrogen, fluoro, (C₁-C₂)alkyl optionally substituted with one to five fluoro, (C₁-C₂)alkoxy optionally substituted with one to five fluoro, —(C═O)—NH₂, —(C═O)—NH—(C₁-C₂)alkyl, —(C═O)—N[(C₁-C₂)alkyl]₂ or —CN;

n is an integer from zero, one, or two;

R³ is (C₁-C₅)alkyl-(C═O)—, [(C₁-C₂)alkyl]₂N—(C═O)—, (C₁-C₅)alkyl-SO₂—, (C₃-C₅)cycloalkyl-SO₂—, or [(C₁-C₂)alkyl]₂N—SO₂—; wherein said (C₁-C₂)alkyl moieties of said [(C₁-C₂)alkyl]₂N—(C═O)— and [(C₁-C₂)alkyl]₂N—SO₂— may optionally be taken together with the nitrogen atom to which they are attached to form a three to six membered heterocyclic ring;

and ring “A” is phenyl, pyridyl, or thienyl.

In yet another embodiment of the present invention the stereochemistry about the piperidone ring is depicted below for Formula IIa:

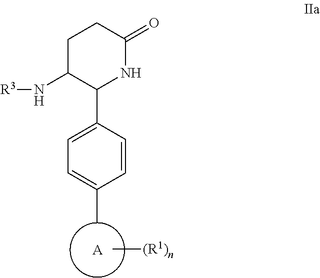

In yet another embodiment of the present invention the stereochemistry about the piperidone ring is depicted below for Formula IIb:

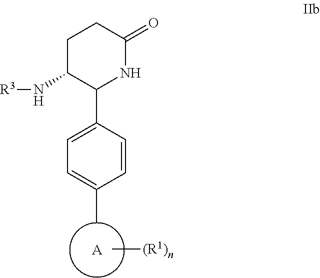

In yet another embodiment of the present invention the stereochemistry about the piperidone ring is depicted below for Formula IIc

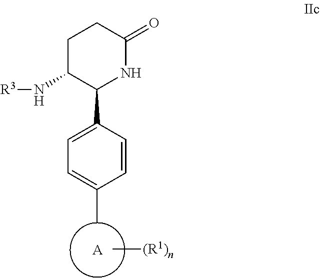

Yet other embodiments of the present invention relate to so called amido piperidones of Formula II (and IIa, IIb, and IIc) wherein R³ is (C₁-C₅)alkyl-(C═O)—.

Yet other embodiments of the present invention relate to so called ureopiperidones of Formula II (and IIa, IIb, and IIc) wherein R³ is [(C₁-C₂)alkyl]₂N—(C═O)—, wherein said (C₁-C₂)alkyl moieties may optionally be taken together with the nitrogen atom to which they are attached to form a four to six membered heterocyclic ring.

Yet other embodiments of the present invention relate to alkylsulfonylpiperidones of Formula II (and IIa, IIb, and IIc) wherein R³ is (C₁-C₅)alkyl-SO₂—.

Yet other embodiments of the present invention relate to cycloalkylsulfonylpiperidones of Formula II (and IIa, IIb, and IIc) wherein R³ is (C₃-C₅)cycloalkyl-SO₂—.

Yet other embodiments of the present invention relate to sulfonamidopiperidones of Formula II (and IIa, IIb, and IIc) wherein R³ is [(C₁-C₂)alkyl]₂N—SO₂—; wherein said (C₁-C₂)alkyl moieties may optionally be taken together with the nitrogen atom to which they are attached to form a four to six membered heterocyclic ring.

Yet other embodiments of interest to the present inventors include compounds of Formula II (and IIa, IIb, and IIc) including the R³ embodiments described above, wherein n is zero or one.

Yet other embodiments of interest to the present inventors include compounds of Formula II (and IIa, IIb, and IIc) including the R³ embodiments described above, wherein n is two.

Yet other embodiments of interest to the present inventors include compounds of Formula II (and IIa, IIb, and IIc) including the R³ embodiments described above and the “n” zero or one and “n” is two embodiments also described above, also including the embodiments comprising combinations of each of the foregoing embodiment groups (e.g. Formula Ia with the R³ sulfonamidopyranes and “n” is two embodiments); wherein one R¹ is fluoro, (C₁-C₂)alkoxy optionally substituted with one to five fluoro (more specifically ethoxy) or —CN (even more specifically wherein this R¹ is an ortho substituent).

Yet other embodiments of interest to the present inventors include compounds of Formula II (and IIa, IIb, and IIc) including the R³ embodiments described above and the “n” is zero or one and “n” is two embodiments also described above, also including the embodiments comprising combinations of each of the foregoing embodiment groups (e.g. Formula IIa with the R³ sulfonamidopiperidones and “n” is two embodiments); wherein one R¹ is (C₁-C₂)alkyl optionally substituted with one to five fluoro.

Exemplary compounds according to the invention include the specific compounds disclosed herein or pharmaceutically acceptable salts thereof.

Another embodiment of interest to the present inventors includes a group of oxopiperidines selected from the group consisting of:
N-[(2S,3R)-2-(2′-ethoxybiphenyl-4-yl)-6-oxopiperidin-3-yl]propane-2-sulfonamide;
N-[(2R,3S)-2-(2′-ethoxybiphenyl-4-yl)-6-oxopiperidin-3-yl]propane-2-sulfonamide;
N-[(2S,3R)-2-biphenyl-4-yl-6-oxopiperidin-3-yl]propane-2-sulfonamide;
N-{(2S,3R)-2-[4-(5-cyano-2-thienyl)phenyl]-6-oxopiperidin-3-yl}propane-2-sulfonamide;
N-{(2R,3S)-2-[4-(5-cyano-2-thienyl)phenyl]-6-oxopiperidin-3-yl}propane-2-sulfonamide; and
N-[(2R,3S)-2-(2′-cyanobiphenyl-4-yl)-6-oxopiperidin-3-yl]propane-2-sulfonamide, or the pharmaceutically acceptable salts thereof.

Other compounds of the invention include:

N-[(2R,3S)-2-biphenyl-4-yl-6-oxopiperidin-3-yl]propane-2-sulfonamide; and N-[(2S,3R)-2-(2′-cyanobiphenyl-4-yl)-6-oxopiperidin-3-yl]propane-2-sulfonamide.

The compounds of Formula I or II are useful for the treatment of a variety of neurological and psychiatric disorders associated with glutamate dysfunction, including: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, social anxiety disorder, panic disorder, post-traumatic stress disorder and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, attention deficit disorder, and conduct disorder. Accordingly, in one embodiment, the invention provides a method for treating a condition in a mammal, such as a human, selected from the conditions above, comprising administering a compound of Formula I or II to the mammal. The mammal is preferably a mammal in need of such treatment or prevention.

The term “treating”, as used herein, unless otherwise indicated, means reversing, alleviating, modulating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

As an example, the invention provides a method for treating a condition selected from migraine, anxiety disorders, schizophrenia, and epilepsy. Exemplary anxiety disorders are generalized anxiety disorder, social anxiety disorder, panic disorder, post-traumatic stress disorder and obsessive-compulsive disorder. As another example, the invention provides a method for treating depression selected from Major Depression, Chronic Depression (Dysthymia), Seasonal Depression (Seasonal Affective Disorder), Psychotic Depression, and Postpartum Depression. As another example, the invention provides a method for treating a sleep disorder selected from insomnia and sleep deprivation.

In another embodiment, the invention comprises methods of treating a condition in a mammal, such as a human, by administering a compound of Formula I or II, wherein the condition is selected from the group consisting of atherosclerotic cardiovascular diseases, cerebrovascular diseases and peripheral arterial diseases, to the mammal. The mammal is preferably a mammal in need of such treatment or prevention. Other conditions that can be treated in accordance with the present invention include hypertension and angiogenesis.

In another embodiment the present invention provides methods of treating neurological and psychiatric disorders associated with glutamate dysfunction, comprising administering to a mammal, preferably a mammal in need thereof, an amount of a compound of Formula I or II effective in treating such disorders.

The compound of Formula I or II is optionally used in combination with another active agent. Such an active agent may be, for example, an atypical antipsychotic or an AMPA potentiator. Accordingly, another embodiment of the invention provides methods of treating neurological and psychiatric disorders associated with glutamate dysfunction, comprising administering to a mammal an amount of a compound of Formula I or II and further comprising administering another active agent.

As used herein, the term “another active agent” refers to any therapeutic agent, other than the compound of Formula (I), or salt thereof, that is useful for the treatment of a subject disorder. Examples of additional therapeutic agents include antidepressants, antipsychotics, anti-pain and anti-anxiety agents. Examples of particular classes of antidepressants that can be used in combination with the compounds of the invention include norepinephrine reuptake inhibitors, selective serotonin reuptake inhibitors (SSRIs), NK-1 receptor antagonists, monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, and atypical antidepressants. Suitable norepinephrine reuptake inhibitors include tertiary amine tricyclics and secondary amine tricyclics. Examples of suitable tertiary amine tricyclics and secondary amine tricyclics include amitriptyline, clomipramine, doxepin, imipramine, trimipramine, dothiepin, butriptyline, iprindole, lofepramine, nortriptyline, protriptyline, amoxapine, desipramine and maprotiline. Examples of suitable selective serotonin reuptake inhibitors include fluoxetine, fluvoxamine, paroxetine, and sertraline. Examples of monoamine oxidase inhibitors include isocarboxazid, phenelzine, and tranylcyclopramine. Examples of suitable reversible inhibitors of monoamine oxidase include moclobemide. Example of suitable serotonin and noradrenaline reuptake inhibitors of use in the present invention include venlafaxine. Examples of suitable atypical anti-depressants include bupropion, lithium, nefazodone, trazodone and viloxazine. Examples of suitable classes of anti-anxiety agents that can be used in combination with the compounds of the invention include benzodiazepines and serotonin 1A (5-HT1A) agonists or antagonists, especially 5-HT1A partial agonists, and corticotropin releasing factor (CRF) antagonists. Suitable benzodiazepines include alprazolam, chlordiazepoxide, clonazepam, chlorazepate, diazepam, halazepam, lorazepam, oxazepam, and prazepam. Suitable 5-HT1A receptor agonists or antagonists include buspirone, flesinoxan, gepirone and ipsapirone. Suitable atypical antipsychotics include paliperidone, bifeprunox, ziprasidone, risperidone, aripiprazole, olanzapine, and quetiapine. Suitable nicotine acetylcholine agonists include ispronicline, varenicline and MEM 3454. Anti-pain agents include pregabalin, gabapentin, clonidine, neostigmine, baclofen, midazolam, ketamine and ziconotide.

The invention is also directed to a pharmaceutical composition comprising a compound of Formula I or II, and a pharmaceutically acceptable carrier.

### DETAILED DESCRIPTION OF THE INVENTION

The compounds of the Formula I and II may be prepared by the methods described below, together with synthetic methods known in the art of organic chemistry, or modifications and derivatisations that are familiar to those of ordinary skill in the art. The starting materials used herein are commercially available or may be prepared by routine methods known in the art (such as those methods disclosed in standard reference books such as the COMPENDIUM OF ORGANIC SYNTHETIC METHODS, Vol. I-VI (published by Wiley-Interscience)). Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1999, which is hereby incorporated by reference.

As appreciated by the artisan, the use of Formula I and II is a convenience and the invention is understood to include each and every species falling thereunder as though individually set forth herein. Thus the invention contemplates each species separately and any and all combinations of such species.

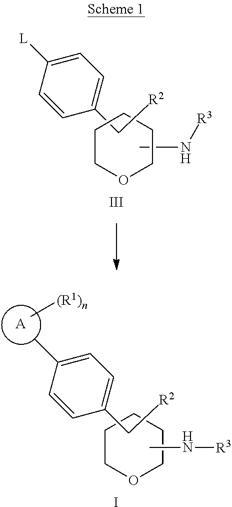

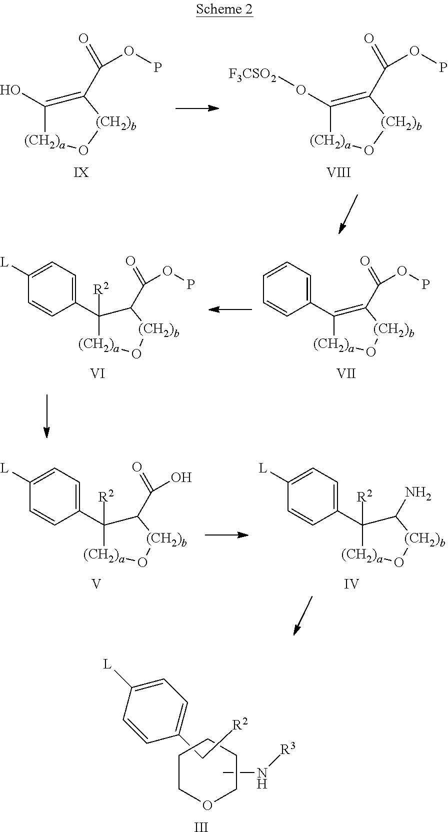

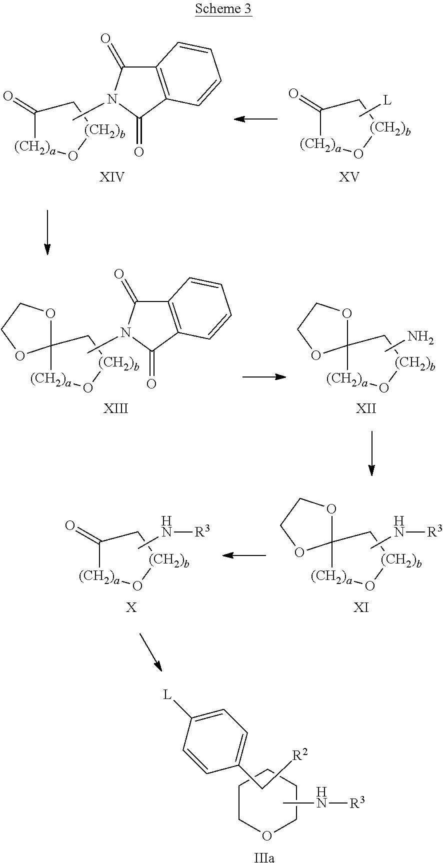

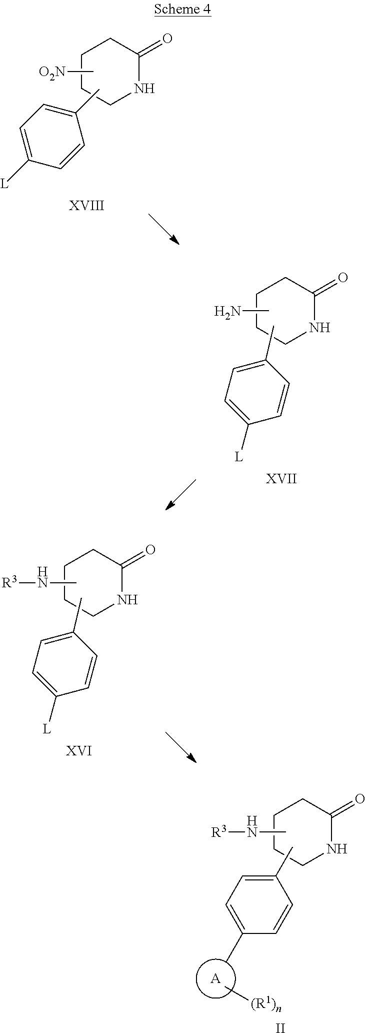

Scheme 1 refers to the preparation of compounds of the Formula I from compounds of Formula III. Compounds of Formula III can be made according to the methods of Schemes 2 and 3. Referring to Scheme 1, the aryl iodinate of Formula III, wherein L is iodo, bromo or a triflate, “a” is an integer from zero to three, and “b” is an integer from zero to three, subject to the proviso that the sum of “a” plus “b” must be three (i.e. the ring is a pyrane ring), can be coupled to a suitably substituted aryl boronic acid of structure ArB(OH)₂, wherein Ar represents a suitably substituted aryl group, under standard palladium catalyzed cross-coupling reaction conditions well known to one of ordinary skill in the art to provide the compound of Formula I. [Suzuki, A., Journal of Organometallic Chemistry, 576, 147-169 (1999), Miyaura and Suzuki, Chemical Reviews, 95, 2457-2483 (1995).] More specifically, the aryl iodinate, bromate or triflate of Formula III is combined with 1 to 3 equivalents of aryl boronic acid and a suitable base, such as 2 to 5 equivalents of potassium carbonate, in a suitable organic solvent such as THF. A palladium catalyst is added, such as 0.02 equivalents of palladium tetrakistriphenylphosphine, and the reaction mixture is heated to temperatures ranging from 60 to 100° C. for 1 to 24 hours. The reaction is not limited to the employment of this solvent, base, or catalyst as many other conditions may be used.

The compound of Formula I can be separated into the enantiomerically pure isomers according to methods well known to those skilled in the art and described in detail in the Example section herein.

Scheme 2 refers to the preparation of compounds of the Formula III.

Referring to Scheme 2, the ketoester of Formula IX, wherein “P” is a protecting group such as alkyl, can be treated with a base such as sodium hydride in diethyl ether, followed by treatment with trifluoromethanesulfonic anhydride to provide the vinyl triflate of Formula VIII. Other non-limiting examples of bases which can be used include hindered amine bases such as triethylamine, diisopropylethylamine, 2,6-lutidine or 2,6-di-tert-butyl-4-methyl pyridine in a suitable solvent, such as dichloromethane.

The vinyl triflate of Formula VIII can be coupled to a suitable aryl boronic acid of structure ArB(OH)₂, wherein Ar represents a suitably substituted aryl group and “L” is hydrogen, under standard palladium catalyzed cross-coupling reaction conditions well known to one of ordinary skill in the art to provide the compound of Formula VII. [Suzuki, A., Journal of Organometallic Chemistry, 576, 147-169 (1999), Miyaura and Suzuki, Chemical Reviews, 95, 2457-2483 (1995).] More specifically, the vinyl triflate of Formula VIII is combined with 1 to 3 equivalents of aryl boronic acid and a suitable base, such as 2 to 5 equivalents of potassium carbonate, in a suitable organic solvent such as THF. A palladium catalyst is added, such as 0.02 equivalents of palladium tetrakistriphenylphosphine, and the reaction mixture is heated to temperatures ranging from 60 to 100° C. for 1 to 24 hours. The reaction is not limited to the employment of this solvent, base, or catalyst as many other conditions may be used.

Alternatively, the vinyl triflate of Formula VIII can be coupled to a suitably substituted Aryl Grignard, wherein “L” is a silyl group (such as trimethylsilyl) in an ethereal solvent such as THF at about −30° C. to about room temperature. A catalyst, such as palladium or copper can facilitate the Rx.

The resultant unsaturated ring of Formula VII can be reduced under conditions well known in the art. For example, treatment of the compound of Formula VII with a palladium catalyst, such as 10% Pd/C, and hydrogen gas at elevated pressure such as 50 psi in a suitable solvent such as ethanol, methanol, or ethyl acetate afford the cis tetrahydropyran product of Formula VI.

The compound of Formula VI, wherein L is hydrogen, may be iodinated or brominated under standard conditions well known to one skilled in the art. For example, the compound of Formula VI may be treated with iodination conditions such as iodine and bis(trifluoroacetoxy)iodobenzene in a solvent such as dichloromethane, chloroform or carbontetrachloride. Alternatively, iodination may be conducted under acidic conditions such as iodine in a mixture of nitric acid and sulfuric acid.

The cis-tetrahydropyrane compounds of Formula VI can be epimerized by treatment with a base, such as sodium ethoxide, using a suitable solvent and temperature, such as ethanol at reflux, to afford the trans-tetrahydropyran ester of Formula VI.

The ester of Formula VI (either cis or trans as desired) can be converted to the carboxylic acid of Formula V under conditions well known in the art. For example, the ester of Formula VI can be treated with excess lithium-, sodium-, or potassium-hydroxide in a suitable solvent such as a mixture of water and methanol, or water, alcohol and THF, at elevated temperatures if necessary. An acidic workup can afford the carboxylic acid of Formula V. Alternatively the compound of Formula VI can be converted into the carboxylic acid under acidic conditions such as hydrochloric acid in water according to methods well known to those skilled in the art.

The carboxylic acid of Formula V can be converted into the primary amine via the Curtius rearrangement under conditions well known in the art. For example, the carboxylic acid of Formula V can be treated with diphenylphosphoryl azide in a suitable solvent such as toluene at elevated temperatures such as 80° C. An organic base such as triethylamine may be added. The crude isocyanate intermediate subsequently may be hydrolyzed using, for example, aqueous hydroxide in combination with an organic solvent such as THF. Alternatively, the isocyanate may be trapped with an organic alcohol such as t-butanol to afford the analogous carbamate. A preferred method involves the treatment of the crude isocyanate with 2 M sodium hydroxide in THF to afford the amine of Formula IV.

The amine of Formula IV can be converted into the various R³ amides, ureas, sulfonamides etc. under conditions well known in the art. For example, a mixture of the amine of Formula IV and a suitable base such as triethylamine or 1,8-diazabicyclo[5.4.0]undec-7-ene can be treated with a sulfonyl chloride in a suitable solvent such as dichloromethane or DMF. Cooling temperatures may be used, such as 0° C.

The compounds of Formula III can be converted into a compound of Formula I according to the methods of Scheme 1.

Scheme 3 refers to an alternate preparation of compounds of Formula IIIa from alpha halo piperidones. The compounds of Formula IIIa can be converted to compounds of Formula I by the methods of Scheme 1. Referring to Scheme 3, alpha halo piperidones of Formula XV, wherein L is halo, (preferably bromo or iodo) can be converted to compounds of the Formula XIV by reaction with a phthalimide, such as potassium phthalimide, in a polar aprotic solvent such as anhydrous tetrahydrofuran (THF) or dimethylformamide (DMF). The reaction is conducted typically at room temperature for 1 to 12 hours.

The compound of the Formula XIV can be converted into a keto protected compound of Formula XIII by reaction with para toluenesulfonic acid in the presence of a glycol (such as ethylene glycol) in a reaction inert solvent such as toluene, methylene chloride, or cyclohexane. Typically the reaction is performed at high temperature, such as the boiling point of the solvent, for from 1 to 5 days.

The compound of Formula XIII can be converted to the free amine of Formula XII by reaction with a hydrazine in a polar solvent such as an alcohol at a temperature of 20 to about 70° C. for a period from about 1 to about 48 hours.

The amine of Formula XII can be derivatized into the various R³ components of Formula XI using methods described above or are well known to those skilled in the art.

The protected ketone of Formula XI can then be liberated to yield the free ketone of Formula X by reaction with an acid (such as PTSA) in an aqueous/organic solvent such as acetone or DMF for from 1 to 48 hrs at a temperature from about 20° C. to about 50° C.

The ketone of Formula X can be converted into the compound of Formula IIIa by reaction with a Grignard reagent as described above in Scheme 2.

Scheme 4 refers to the preparation of compounds of the Formula II. Referring to Scheme 4, a compound of Formula II can be prepared from nitropiperidones of Formula XVIII. Specifically, the Compound of Formula XVIII can be reduced to a compound of Formula XVII by reaction with a reducing agent such as palladium on carbon in a polar solvent at room temperature for from one to about 12 hours.

The compound of Formula XVII can be converted into a R³ derivatized compound of Formula XVI by methods well known to those skilled in the art and discussed above in Schemes 2 and 3.

The compound of Formula XVI can be converted into a compound of Formula II by reaction with a suitably substituted aryl boronic acid of structure ArB(OH)₂, wherein Ar represents a suitably substituted aryl group, under standard palladium catalyzed cross-coupling reaction conditions well known to one of ordinary skill in the art and described above in Scheme 1.

The compounds of Formula XVIII, XV, and IX are commercially available or can be made by literature methods.

The compounds of the invention can be isolated as salts. Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

The compounds of the invention are intended to be administered in an amount effective to treat or prevent a condition as described herein. The compounds of the invention are administered by any suitable route in the form of a pharmaceutical composition adapted to such a route, and in a dose effective for the treatment or prevention intended. Therapeutically effective doses of the compounds required to treat or prevent the progress of the medical condition are readily ascertained by one of ordinary skill in the art using preclinical and clinical approaches familiar to the medicinal arts.

The compounds of the invention may be administered orally. Oral administration may involve swallowing, so that the compound enters the gastrointestinal tract, or buccal or sublingual administration may be employed by which the compound enters the blood stream directly from the mouth.

In another embodiment, the compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment or prevention of the above-indicated conditions. In one embodiment, the total daily dose of a compound of the invention (administered in single or divided doses) is typically from about 0.01 to about 100 mg/kg. In another embodiment, total daily dose of the compound of the invention is from about 0.1 to about 50 mg/kg, and in another embodiment, from about 0.5 to about 30 mg/kg (i.e., mg compound of the invention per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration, the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

In another embodiment, the invention comprises the use of one or more compounds of the invention for the preparation of a medicament for the treatment or prevention of the conditions recited herein.

For the treatment or prevention of the conditions referred to above, the compound of the invention can be administered as compound per se. Alternatively, pharmaceutically acceptable salts are suitable for medical applications because of their greater aqueous solubility relative to the parent compound.

In another embodiment, the present invention comprises pharmaceutical compositions. Such pharmaceutical compositions comprise a compound of the invention presented with a pharmaceutically-acceptable carrier. The carrier can be a solid, a liquid, or both, and may be formulated with the compound as a unit-dose composition, for example, a tablet, which can contain from 0.05% to 95% by weight of the active compounds. A compound of the invention may be coupled with suitable polymers as targetable drug carriers. Other pharmacologically active substances can also be present.

The compounds of the present invention may be administered by any suitable route, preferably in the form of a pharmaceutical composition adapted to such a route, and in a dose effective for the treatment or prevention intended. The active compounds and compositions, for example, may be administered orally, rectally, parenterally, or topically.

Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sub-lingual, such as, for example, a lozenge. In such solid dosage forms, the compounds of Formula I or II are ordinarily combined with one or more adjuvants. Such capsules or tablets may contain a controlled-release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the present invention comprises a parenteral dose form. “Parenteral administration” includes, for example, subcutaneous injections, intravenous injections, intraperitoneally, intramuscular injections, intrasternal injections, and infusion. Injectable preparations (e.g., sterile injectable aqueous or oleaginous suspensions) may be formulated according to the known art using suitable dispersing, wetting agents, and/or suspending agents.

In another embodiment, the present invention comprises a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Formulations suitable for topical administration to the eye include, for example, eye drops wherein the compound of this invention is dissolved or suspended in suitable carrier. A typical formulation suitable for ocular or aural administration may be in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

The present invention further comprises kits that are suitable for use in performing the methods of treatment or prevention described above. In one embodiment, the kit contains a first dosage form comprising one or more of the compounds of the present invention and a container for the dosage, in quantities sufficient to carry out the methods of the present invention.

In another embodiment, the kit of the present invention comprises one or more compounds of the invention.

### EXPERIMENTAL PROCEDURES

Experiments were generally carried out under inert atmosphere (nitrogen or argon) particularly in cases where oxygen or moisture sensitive reagents or intermediates were employed. Commercial solvents and reagents were generally used without further purification, including anhydrous solvents where appropriate (generally Sure-Seal™ products from the Aldrich Chemical Company, Milwaukee, Wis.). Chemical shifts for nuclear magnetic resonance (NMR) data are expressed in parts per million (ppm, δ) referenced to residual peaks from the deuterated solvents employed.

### Example 1

### N-{(3S,4S)-3-[4-(5-cyano-2-thienyl)phenyl]tetrahydro-2H-pyran-4-yl}propane-2-sulfonamide

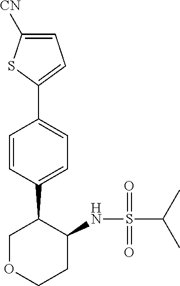

Step 1: Preparation of ethyl 4-(2-ethoxy-2-oxoethoxy)butanoate. A flask was charged with ethyl 4-hydroxybutanoate (H. Smith and R. Jones, Synthetic Communications, 1994, 24, 2743-2747) (19.08 g, 100 mMol), rhodium(II) acetate (445 mg, 1.01 mMol) and methylene chloride (400 mL). Ethyl diazoacetate (18.1 mL, 152 mMol) in methylene chloride (100 mL) was added drop-wise via an addition funnel over a period of 1.5 hours and the reaction was stirred at room temperature for 72 hours. The reaction mixture was filtered through a plug of silica gel, the plug was washed with methylene chloride, and the filtrate was concentrated to a light yellow oil. The crude oil was purified by fractional distillation at reduced pressure. The fraction boiling between 70-80° C. at ˜1 torr was collected to give ethyl 4-(2-ethoxy-2-oxoethoxy)butanoate as a clear oil. Yield: 19.20 g, 87 mMol, 87%. ¹H NMR (400 MHz, CHLOROFORM-d) δ 1.22-1.29 (m, 6H), 1.89-1.97 (m, 2H), 2.43 (t, 2H), 3.56 (t, 2H), 4.04 (s, 2H), 4.12 (q, 2H), 4.20 (q, 2H).

Step 2: Preparation of ethyl 5-hydroxy-3,6-dihydro-2H-pyran-4-carboxylate. A flask was charged with ethyl 4-(2-ethoxy-2-oxoethoxy)butanoate (15.01 g, 68.77 mMol) and toluene (300 mL). A solution of potassium tert-butoxide solution in tetrahydrofuran (1.0 M, 82.0 mL, 82 mMol) was added to the reaction via syringe over the course of 10 minutes at room temperature and stirred for 18 hours. The reaction was poured into 1.0 N hydrochloric acid (300 mL), the organic phase was separated and the aqueous phase was extracted with ether. The combined organic phases were washed with water, saturated aqueous sodium chloride solution, dried over sodium sulfate, filtered and concentrated to give an orange oil. The crude oil was purified by silica gel chromatography (Eluant: 5% ethyl acetate in heptane). The combined product fractions were concentrated to give ethyl 5-hydroxy-3,6-dihydro-2H-pyran-4-carboxylate as a light yellow oil. Yield: 7.21 g, 41.9 mMol, 61%. ¹H NMR (400 MHz, CHLOROFORM-d) δ 1.32 (t, 3H), 2.33-2.37 (m, 2H), 3.79 (t, 2H), 4.12-4.15 (m, 2H), 4.25 (q, 2H), 11.85 (s, 1H).

Step 3: Preparation of ethyl 5-{[(trifluoromethyl)sulfonyl]oxy}-3,6-dihydro-2H-pyran-4-carboxylate. A 125 mL 3-neck round bottom flask equipped with a magnetic stir bar, septum and nitrogen blanket was charged with sodium hydride (60% suspension in mineral oil, 275 mg, 6.88 mMol). The sodium hydride was washed with heptane (2×20 mL) and the flask was charged with anhydrous diethyl ether (30 mL). To the stirring suspension was added drop-wise a solution of ethyl 5-hydroxy-3,6-dihydro-2H-pyran-4-carboxylate (1.03 g, 5.98 mMol) in anhydrous ether (5 mL) and the mixture was allowed to stir at room temperature for 1 hour. To the reaction was added trifluoromethanesulfonic anhydride (1.01 mL, 5.98 mMol) and stirring was continued at room temperature overnight. The reaction was quenched with saturated aqueous ammonium chloride solution, the organic layer was separated, washed with saturated aqueous sodium chloride solution, dried over magnesium sulfate, filtered, and concentrated under reduced pressure to give ethyl 5-{[(trifluoromethyl)sulfonyl]oxy}-3,6-dihydro-2H-pyran-4-carboxylate as a yellow oil, which was used without further purification. Yield: 1.63 g, 5.3 mMol, 89%. ¹H NMR (400 MHz, CHLOROFORM-d) δ 1.35 (t, J=7.0 Hz, 3H), 2.61-2.66 (m, 2H), 3.82 (t, J=5.4 Hz, 2H), 4.19 (t, J=2.7 Hz, 2H), 4.32 (q, J=7.1 Hz, 2H).

Step 4: Preparation of ethyl 5-phenyl-3,6-dihydro-2H-pyran-4-carboxylate. A flask was charged with ethyl 5-{[(trifluoromethyl)sulfonyl]oxy}-3,6-dihydro-2H-pyran-4-carboxylate (25.5 g, 83.82 mMol), phenylboronic acid (11.50 g, 92.4 mMol), potassium carbonate (27.89 g, 201.8 mMol), and tetrahydrofuran (430 mL), and evacuated and back filled with nitrogen three times. Tetrakis(triphenylphosphine)palladium(0) (2.71 g, 2.35 mMol) was added and the flask was evacuated and back filled with nitrogen twice. The reaction was heated to 65° C. for 20 hours, at which point a second portion of phenylboronic acid (3.13 g, 25.15 mMol) was added and heating continued for another 20 hours. The cooled reaction was partitioned between ethyl acetate (500 mL) and water (500 mL). The aqueous layer was re-extracted with ethyl acetate (250 mL) and the combined organic layers were washed with saturated aqueous sodium bicarbonate solution, saturated aqueous sodium chloride solution, dried over magnesium sulfate, filtered, and concentrated to give a dark red slurry. The slurry was triturated with a 1:4 ethyl acetate:heptane solution and filtered; the filtrate was concentrated to a light red slurry that was purified by silica gel chromatography (Gradient: 0 to 20% ethyl acetate in heptane) to produce ethyl 5-phenyl-3,6-dihydro-2H-pyran-4-carboxylate as a light yellow oil. Yield: 18.39 g, 79.18 mMol, 94%. LCMS m/z 233.1 (M+1). ¹H NMR (400 MHz, CHLOROFORM-d) δ 0.88 (t, J=7.3 Hz, 3 H), 2.56 (m, 2H), 3.91 (m, 4H), 4.31 (t, J=2.7 Hz, 2H), 7.15 (m, 2H), 7.33 (m, 3H).

Step 5: Preparation of ethyl cis-3-phenyltetrahydro-2H-pyran-4-carboxylate. A 500 mL Parr bottle was charged with ethyl 5-phenyl-3,6-dihydro-2H-pyran-4-carboxylate (12.2 g, 52.52 mMol), absolute ethanol (180 mL), and palladium on activated carbon catalyst (10% wt/wt, 2.78 g) and shaken under an atmosphere of hydrogen (50 psi) for 3 hours. The catalyst was removed by filtration and the filtrate concentrated to give the product ethyl cis-3-phenyltetrahydro-2H-pyran-4-carboxylate as a clear oil. Yield: 11.87 g, 50.7 mMol, 96%. LCMS m/z 235.2 (M+1). ¹H NMR (400 MHz, CHLOROFORM-d) δ 1.07 (t, J=7.2 Hz, 3H), 1.77 (m, 1H), 2.07 (m, 1H), 3.00 (m, 1H), 3.22 (m, 1H), 3.63 (m, 1H), 3.89 (dd, J=11.4, 3.5 Hz, 1H), 3.96 (m, 2H), 4.15 (m, 1H), 4.29 (dd, J=11.4, 4.8 Hz, 1H), 7.25 (m, 3H), 7.39 (d, J=6.8 Hz, 2H).

Step 6: Preparation of ethyl cis-3-(4-iodophenyl)tetrahydro-2H-pyran-4-carboxylate. A vial was charged with ethyl cis-3-phenyltetrahydro-2H-pyran-4-carboxylate (234.1 mg, 0.999 mMol), glacial acetic acid (0.91 mL), and concentrated sulfuric acid (0.12 mL). Iodine (121 mg, 0.477 mMol), and sodium iodate (40.6 mg, 0.205 mMol) were added and the reaction was stirred vigorously at 70° C. for 20 hours. Sodium periodate (10 mg, 0.047 mMol) was added and stirring continued at 70° C. until the reaction turned from brownish purple to orange. The acetic acid was removed at reduced pressure and the residue diluted with water and extracted with methylene chloride three times. The combined organics were washed with water three times, washed with saturated aqueous sodium chloride solution, and dried over magnesium sulfate. Filtration and concentration in vacuo provided ethyl cis-3-(4-iodophenyl)tetrahydro-2H-pyran-4-carboxylate as a light orange oil, which was generally used in the next step without purification. Yield: 367.0 mg, assumed quantitative. An analytical sample was purified using silica gel chromatography (Gradient: 0 to 20% ethyl acetate in heptane). LCMS m/z 361.0 (M+1). ¹H NMR (400 MHz, CHLOROFORM-d) δ 1.12 (t, J=7.1 Hz, 3 H), 1.75 (m, 1H), 2.04 (m, 1H), 2.98 (m, 1H), 3.17 (m, 1H), 3.60 (m, 1H), 3.85 (dd, J=11.6, 3.5 Hz, 1H), 3.98 (m, 2H), 4.13 (m, 1H), 4.23 (dd, J=11.6, 4.2 Hz, 1H), 7.18 (d, J=8.7 Hz, 2H), 7.60 (d, J=8.3 Hz, 2H).

Step 7: Preparation of cis-3-(4-iodophenyl)tetrahydro-2H-pyran-4-carboxylic acid. A mixture of ethyl cis-3-(4-iodophenyl)tetrahydro-2H-pyran-4-carboxylate (7.695 g, 21.36 mMol), 3 N aqueous hydrochloric acid solution (210 mL), and 1,4-dioxane (70 mL) was heated to reflux overnight. The reaction was concentrated and the aqueous layer extracted three times with ethyl acetate. The combined organics were washed with water, washed with saturated aqueous sodium chloride solution, and dried over magnesium sulfate. Filtration and concentration in vacuo using a toluene azeotrope provided cis-3-(4-iodophenyl)tetrahydro-2H-pyran-4-carboxylic acid as a viscous brown oil which was generally used in the next step without purification. Yield: 7.36 g, assumed quantitative. ¹H NMR (400 MHz, CHLOROFORM-d) δ 1.75 (m, 1H), 2.03 (m, 1H), 3.03 (m, 1H), 3.18 (m, 1 H), 3.60 (m, 1H), 3.86 (dd, J=11.6, 3.3 Hz, 1H), 4.13 (m, apparent dt, J=11.6, 4.0 Hz, 1H), 4.23 (dd, J=11.6, 3.5 Hz, 1H), 7.22 (d, J=8.3 Hz, 2H), 7.60 (d, J=8.5 Hz, 2H).

Step 8: Preparation of cis-3-(4-iodophenyl)tetrahydro-2H-pyran-4-amine. To a solution of cis-3-(4-iodophenyl)tetrahydro-2H-pyran-4-carboxylic acid (7.36 g, 22.2 mMol) in anhydrous toluene (100 mL) was added diisopropylethylamine (5.79 mL, 33.2 mMol) and diphenylphosphoryl azide (DPPA) (7.41 mL, 33.2 mMol). The reaction was stirred at 85° C. for 5 hours, then allowed to come to room temperature. Tetrahydrofuran (100 mL) and 2 N aqueous sodium hydroxide solution (50 mL) were added and the reaction was stirred vigorously overnight. The layers were separated and the aqueous phase extracted twice with ethyl acetate. The combined organics were washed twice with water then extracted with 1 N aqueous hydrochloric acid solution. The combined acidic extracts were basified to pH 12 with 2 N aqueous sodium hydroxide solution and extracted three times with methylene chloride. The combined organics were dried with magnesium sulfate, filtered and concentrated to give cis-3-(4-iodophenyl)tetrahydro-2H-pyran-4-amine as a yellow oil of sufficient purity for use in the next step without further purification. Yield: 5.19 g, 17.1 mMol, 77%. LCMS m/z 304.0 (M+1) ¹H NMR (400 MHz, CHLOROFORM-d) δ 1.05 (br s, 2H), 1.60 (m, 1H), 1.84 (m, 1H), 2.94 (m, 1H), 3.32 (m, 1H), 3.66 (m, 1H), 3.80 (dd, J=11.4, 3.5 Hz, 1H), 4.00 (m, 1H), 4.17 (dd, J=11.4, 6.9 Hz, 1H), 7.12 (d, J=8.3 Hz, 2H), 7.65 (d, J=8.3 Hz, 2H).

Step 9: Preparation of cis-N-[3-(4-iodophenyl)tetrahydro-2H-pyran-4-yl]propane-2-sulfonamide. A flask was charged with a mixture of cis-3-(4-iodophenyl)tetrahydro-2H-pyran-4-amine (2.46 g, 8.11 mMol), 1,8-diazabicyclo[5.4.0]undec-7-ene (DBU) (2.48 mL, 16.3 mMol), and methylene chloride (40 mL), and placed in a −15° C. cooling bath. Propane-2-sulfonyl chloride (1.95 mL, 16.5 mMol) in methylene chloride (5 mL) was added and the reaction stirred at −15° C. for 30 minutes, at which time LCMS showed mostly product. The reaction was warmed to room temperature, washed with saturated aqueous sodium bicarbonate solution, dried, and concentrated to a light yellow foam. The crude product was purified by silica gel chromatography (Gradient: 20 to 50% ethyl acetate in heptane) to provide cis-N-[3-(4-iodophenyl)tetrahydro-2H-pyran-4-yl]propane-2-sulfonamide as a white foam. Yield: 2.26 g, 5.53 mMol, 68%. LCMS m/z 408.0 (M−1). ¹H NMR (400 MHz, METHANOL-d₄) δ 1.09 (d, J=6.6 Hz, 3H), 1.18 (d, J=6.8 Hz, 3H), 1.83 (m, 2H), 2.92 (m, 1H), 3.09 (m, 1H), 3.71 (m, 1H), 3.85 (m, 2H), 3.98 (m, 1H), 4.09 (dd, J=12.0, 5.8 Hz, 1H), 7.26 (d, J=8.3 Hz, 2H), 7.66 (d, J=8.3 Hz, 2H).

Step 10: Isolation of N-[(3R,4R)-3-(4-iodophenyl)tetrahydro-2H-pyran-4-yl]propane-2-sulfonamide and N-[(3S,4S)-3-(4-iodophenyl)tetrahydro-2H-pyran-4-yl]propane-2-sulfonamide. The racemic material cis-N-[3-(4-iodophenyl)tetrahydro-2H-pyran-4-yl]propane-2-sulfonamide (2.26 g, 5.52 mMol) was subjected to chiral HPLC chromatography using a Chiralcel OJ-H column, 5 μm, 2.1 cm×25 cm, mobile phase: 70/30 mixture of carbon dioxide/methanol at a flow rate of 65 g/minute.

Material with a retention time of 1.64 minutes was collected to afford N-[(3R,4R)-3-(4-iodophenyl)tetrahydro-2H-pyran-4-yl]propane-2-sulfonamide as a white foam. Yield: 0.834 mg, 2.04 mMol, 37%. LCMS m/z 408 (M−1). ¹H NMR (400 MHz, METHANOL-d₄) δ 1.09 (d, J=6.6 Hz, 3H), 1.17 (d, J=6.8 Hz, 3H), 1.81 (m, 2H), 2.92 (m, 1H), 3.09 (m, 1H), 3.71 (m, 1H), 3.85 (m, 2H), 3.98 (m, 1H), 4.09 (dd, J=11.9, 5.9 Hz, 1H), 7.26 (d, J=8.3 Hz, 2H), 7.66 (d, J=8.3 Hz, 2H). The absolute configuration of N-[(3R,4R)-3-(4-iodophenyl)tetrahydro-2H-pyran-4-yl]propane-2-sulfonamide was established via X-ray crystallography.

Material with a retention time of 2.05 minutes was collected to yield N-[(3S,4S)-3-(4-iodophenyl)tetrahydro-2H-pyran-4-yl]propane-2-sulfonamide as a white foam. Yield: 0.814 mg, 1.99 mMol, 36%. LCMS m/z 408 (M−1). ¹H NMR (400 MHz, METHANOL-d₄) δ 1.10 (d, J=6.6 Hz, 3H), 1.18 (d, J=6.8 Hz, 3H), 1.82 (m, 2H), 2.93 (m, 1H), 3.09 (m, 1H), 3.70 (m, 1H), 3.85 (m, 2H), 3.98 (m, 1H), 4.09 (dd, J=11.8, 5.8 Hz, 1H), 7.26 (d, J=8.3 Hz, 2H), 7.66 (d, J=8.5 Hz, 2H).

Step 11: Preparation of N-{(3S,4S)-3-[4-(5-cyano-2-thienyl)phenyl]tetrahydro-2H-pyran-4-yl}propane-2-sulfonamide. A vial was charged with a mixture of N-[(3S,4S)-3-(4-iodophenyl)tetrahydro-2H-pyran-4-yl]propane-2-sulfonamide (400 mg, 0.98 mMol), 2-dicyclohexylphosphino-2′,4′,6′-triisopropyl-1,1′-biphenyl (X-phos) (45.3 mg, 0.095 mMol), potassium fluoride (287 mg, 4.94 mMol), (5-cyano-2-thienyl)boronic acid (224 mg, 1.47 mMol) and palladium(II) acetate (14.6 mg, 0.065 mMol) and evacuated and backfilled with nitrogen three times. A degassed 1:1 mixture of methanol and toluene (4.8 mL) was added and the reaction was heated to 130° C. in a microwave reactor for 35 minutes. The solvent was evaporated and the residue partitioned between water and ethyl acetate. The aqueous layer was extracted twice with ethyl acetate and the combined organic extracts were washed with saturated aqueous sodium chloride solution and dried over magnesium sulfate. Filtration and concentration in vacuo provided crude product, which was purified by preparative silica gel thin layer chromatography (Eluant: 40% ethyl acetate in heptane) to provide the title compound as a pale yellow oil. Yield: 0.14 g, 0.358 mMol, 37%. LCMS m/z 389 (M−1). ¹H NMR (400 MHz, CHLOROFORM-d) δ 1.24 (d, J=6.6 Hz, 3H), 1.29 (d, J=6.8 Hz, 3H), 1.82 (m, 2H), 3.04 (m, 1H), 3.15 (m, 1H), 3.64 (m, 1 H), 3.85 (m, 2H), 3.95 (d, J=9.8 Hz, 1H), 4.07 (m, 1H), 4.17 (dd, J=12.0, 2.9 Hz, 1H), 7.26 (d, J=3.7 Hz, 1H), 7.56 (d, J=8.3 Hz, 2H), 7.57 (d, J=3.9 Hz, 1H), 7.63 (d, J=8.3 Hz, 2H).

### Example 2

### N-[(3R,4S)-3-biphenyl-4-yltetrahydro-2H-pyran-4-yl]propane-2-sulfonamide

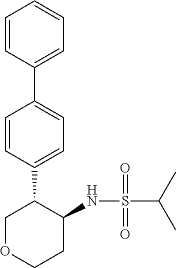

Step 1: Preparation of ethyl trans-3-phenyltetrahydro-2H-pyran-4-carboxylate. Ethyl cis-3-phenyltetrahydro-2H-pyran-4-carboxylate (5.95 g, 25.4 mMol), absolute ethanol (125 mL), and 21 weight % sodium ethoxide in ethanol (10.4 mL, 27.9 mMol) were combined and heated to reflux for 18 hours. The reaction was cooled to room temperature, concentrated, acidified with saturated aqueous ammonium chloride solution, and extracted three times with methylene chloride. The organics were dried and concentrated to give ethyl trans-3-phenyltetrahydro-2H-pyran-4-carboxylate as a reddish brown oil of sufficient purity for use in the next step without further purification. Yield: 5.21 g, 22.2 mMol, 87%. LCMS m/z 235.1 (M+1). ¹H NMR (400 MHz, CHLOROFORM-d) δ 0.99 (t, J=7.2 Hz, 3H), 1.96 (m, 2H), 2.85 (ddd, J=11.1, 11.1, 4.9 Hz, 1H), 3.11 (ddd, J=11.3, 11.3, 4.5 Hz, 1H), 3.39 (dd, apparent t, J=11.4, 11.4 Hz, 1H), 3.52 (m, 1H), 3.94 (q, J=7.1 Hz, 2H), 3.97 (dd, J=11.5, 4.3 Hz, 1H), 4.09 (m, 1H), 7.22 (m, 3H), 7.28 (m, 2H).

Step 2: Preparation of ethyl trans-3-(4-iodophenyl)tetrahydro-2H-pyran-4-carboxylate. Ethyl trans-3-phenyltetrahydro-2H-pyran-4-carboxylate (5.21 g, 22.2 mMol), iodine (3.10 g, 12.2 mMol) and methylene chloride (110 mL) were combined and stirred 30 minutes, at which time the iodine had dissolved. [Bis(trifluoroacetoxy)iodo]benzene (6.55 g, 14.8 mMol) was added and the reaction stirred for three hours. Aqueous sodium thiosulfate solution (100 mL of 10% wt solution) and water (50 mL) were added and the mixture stirred vigorously until the purple color was no longer present. The phases were separated and the aqueous layer was extracted with methylene chloride. The organics were dried, concentrated, and purified by silica gel chromatography (Gradient: 0 to 15% ethyl acetate in heptane) to give the product ethyl trans-3-(4-iodophenyl)tetrahydro-2H-pyran-4-carboxylate as a yellow oil. Yield: 7.02 g, 19.46 mMol, 88%. LCMS m/z 361.0 (M+1). ¹H NMR (400 MHz, CHLOROFORM-d) δ 1.05 (t, J=7.2 Hz, 3H), 1.94 (m, 2H), 2.80 (m, 1H), 3.07 (ddd, J=11.2, 11.2, 4.4 Hz, 1H), 3.34 (dd, apparent t, J=11.4, 11.4 Hz, 1H), 3.51 (m, 1H), 3.92 (dd, J=11.5, 4.5 Hz, 1H), 3.96 (q, J=7.2 Hz, 2H), 4.09 (m, 1H), 6.97 (d, J=8.3 Hz, 2H), 7.62 (d, J=8.3 Hz, 2H).

Step 3: Preparation of trans-3-(4-iodophenyl)tetrahydro-2H-pyran-4-carboxylic acid. Ethyl trans-3-(4-iodophenyl)tetrahydro-2H-pyran-4-carboxylate (6.99 g, 19.4 mMol), 2.0 N aqueous sodium hydroxide solution (30 mL, 60 mMol) and methanol (60 mL) were combined and heated to 70° C. overnight. The reaction was concentrated and the residue partitioned between water (˜150 mL) and ether (˜50 mL). The aqueous layer was acidified to pH 1 with concentrated hydrochloric acid and extracted three times with methylene chloride. The combined organics were dried over magnesium sulfate, filtered, and concentrated in vacuo to give trans-3-(4-iodophenyl)tetrahydro-2H-pyran-4-carboxylic acid as a white foam of sufficient purity to use in the next step. Yield: 6.47 g, quantitative. ¹H NMR (400 MHz, CHLOROFORM-d) δ 1.97 (m, 2H), 2.83 (ddd, J=11.4, 11.4, 4.3 Hz, 1H), 3.05 (ddd, J=11.2, 11.2, 4.4 Hz, 1H), 3.31 (dd, apparent t, J=11.4, 11.4 Hz, 1H), 3.51 (m, 1H), 3.91 (dd, J=11.6, 4.4 Hz, 1H), 4.10 (m, 1H), 6.97 (d, J=8.3 Hz, 2H), 7.63 (d, J=8.3 Hz, 2H).

Step 4: Preparation of trans-3-(4-iodophenyl)tetrahydro-2H-pyran-4-amine. To a solution of trans-3-(4-iodophenyl)tetrahydro-2H-pyran-4-carboxylic acid (6.47 g, 19.5 mMol) in anhydrous toluene (90 mL) was added diisopropylethylamine (5.09 mL, 29.2 mMol) and diphenylphosphoryl azide (DPPA) (6.51 mL, 29.2 mMol). The reaction was stirred at 85° C. for 5 hours, then allowed to come to room temperature. Tetrahydrofuran (100 mL) and 2 N aqueous sodium hydroxide solution (50 mL) were added and the reaction stirred vigorously overnight. The layers were separated and the aqueous phase extracted twice with ethyl acetate. The combined organics were washed twice with water, once with saturated aqueous sodium chloride solution, and dried over magnesium sulfate. Filtration and concentration in vacuo gave the product, trans-3-(4-iodophenyl)tetrahydro-2H-pyran-4-amine, as a yellow oil which was generally used in the next step without further purification. Yield: 6.03 g, assumed quantitative. LCMS m/z 304.0 (M+1). ¹H NMR (400 MHz, CHLOROFORM-d) δ 1.60 (m, 1H), 1.92 (m, 1H), 2.51 (ddd, J=10.7, 10.7, 4.5 Hz, 1H), 3.06 (ddd, J=10.6, 10.6, 4.2 Hz, 1H), 3.34 (dd, apparent t, J=11.4, 11.4 Hz, 1H), 3.57 (ddd, J=12.1, 12.1, 2.2 Hz, 1H), 3.88 (dd, J=11.5, 4.3 Hz, 1H), 4.09 (br dd, J=11.6, 4.6 Hz, 1H), 6.99 (d, J=8.3 Hz, 2H), 7.67 (d, J=8.3 Hz, 2H).

Step 5: Preparation of trans-N-[3-(4-iodophenyl)tetrahydro-2H-pyran-4-yl]propane-2-sulfonamide. A flask was charged with a mixture of trans-3-(4-iodophenyl)tetrahydro-2H-pyran-4-amine (3.231 g, 10.66 mMol), 1,8-diazabicyclo[5.4.0]undec-7-ene (DBU) (3.25 mL, 21.3 mMol), and methylene chloride (40 mL), and placed in a −15° C. cooling bath. Propane-2-sulfonyl chloride (2.47 mL, 21.4 mMol) in methylene chloride (5 mL) was added and the reaction stirred at −15° C. for 30 minutes, at which time LCMS showed nearly complete conversion to product. The reaction was stirred an additional 30 minutes at −15° C. and then allowed to warm to room temperature. The reaction was washed with saturated aqueous sodium bicarbonate solution; the organic layer was dried over magnesium sulfate, filtered, and concentrated to a light yellow oil. The crude product was purified by silica gel chromatography (Gradient: 20 to 50% ethyl acetate in heptane) to provide the product, trans-N-[3-(4-iodophenyl)tetrahydro-2H-pyran-4-yl]propane-2-sulfonamide, as a white foam. Yield: 2.18 g, 5.33 mMol, 50%. LCMS m/z 408.0 (M−1). ¹H NMR (400 MHz, METHANOL-d₄) δ 0.75 (d, J=6.6 Hz, 3H), 1.08 (d, J=6.9 Hz, 3H), 1.74 (m, 1H), 2.14 (m, 1H), 2.52 (m, 1H), 2.71 (ddd, J=11.2, 11.2, 4.6 Hz, 1H), 3.50 (dd, apparent t, J=11.4, 11.4 Hz, 1H), 3.60 (m, 2H), 3.86 (br dd, J=11.6, 4.2 Hz, 1H), 3.99 (br dd, J=11.6, 4.6 Hz, 1H), 7.12 (d, J=8.3 Hz, 2H), 7.68 (d, J=8.3 Hz, 2H).

Step 6: Isolation of N-[(3R,4S)-3-(4-iodophenyl)tetrahydro-2H-pyran-4-yl]propane-2-sulfonamide and N-[(3S,4R)-3-(4-iodophenyl)tetrahydro-2H-pyran-4-yl]propane-2-sulfonamide. The racemic material trans-N-[3-(4-iodophenyl)tetrahydro-2H-pyran-4-yl]propane-2-sulfonamide (2.0 g, 4.9 mMol) was subjected to chiral HPLC chromatography using a Chiralpak AS-H column, 5 μm, 2.1 cm×25 cm, mobile phase: 80/20 mixture of carbon dioxide/methanol at a flow rate of 65 g/minute.

Material with a retention time of 3.02 minutes was collected to yield N-[(3R,4S)-3-(4-iodophenyl)tetrahydro-2H-pyran-4-yl]propane-2-sulfonamide. Yield: 899 mg, 2.20 mMol, 45%. APCI MS m/z 407.5 (M−1). ¹H NMR (400 MHz, METHANOL-d₄) δ 0.75 (d, J=6.6 Hz, 3H), 1.08 (d, J=6.8 Hz, 3H), 1.74 (m, 1H), 2.14 (m, 1H), 2.52 (m, 1H), 2.71 (ddd, J=11.2, 11.2, 4.6 Hz, 1H), 3.50 (dd, apparent t, J=11.4, 11.4 Hz, 1H), 3.60 (m, 2H), 3.86 (dd, J=11.6, 4.6 Hz, 1H), 3.99 (br dd, J=11.6, 4.6 Hz, 1H), 7.12 (d, J=8.3 Hz, 2H), 7.68 (d, J=8.1 Hz, 2H). The absolute configuration of this material was established via X-ray crystallography.

Material with a retention time of 2.38 minutes was collected to yield N-[(3S,4R)-3-(4-iodophenyl)tetrahydro-2H-pyran-4-yl]propane-2-sulfonamide. Yield: 910 mg, 2.23 mMol, 45%. ¹H NMR (400 MHz, METHANOL-d₄) δ 0.75 (d, J=6.8 Hz, 3H), 1.08 (d, J=6.8 Hz, 3H), 1.74 (m, 1H), 2.14 (m, 1H), 2.52 (m, 1H), 2.71 (ddd, J=11.1, 11.1, 4.6 Hz, 1H), 3.50 (dd, apparent t, J=11.4, 11.4 Hz, 1H), 3.60 (m, 2H), 3.86 (dd, J=11.6, 4.4 Hz, 1H), 3.99 (br dd, J=11.6, 4.6 Hz, 1H), 7.12 (d, J=8.3 Hz, 2H), 7.68 (d, J=8.3 Hz, 2H).

Step 7: Preparation of N-[(3R,4S)-3-biphenyl-4-yltetrahydro-2H-pyran-4-yl]propane-2-sulfonamide. A mixture of N-[(3R,4S)-3-(4-iodophenyl)tetrahydro-2H-pyran-4-yl]propane-2-sulfonamide (400 mg, 0.977 mMol), phenylboronic acid (131 mg, 1.08 mMol), tetrakis(triphenylphosphine)palladium(0) (56.6 mg, 0.049 mMol) sodium carbonate (518 mg, 4.88 mMol), degassed methanol (8.0 mL), degassed toluene (1.0 mL), and degassed water (1.0 mL) was evacuated and backfilled with nitrogen three times. The reaction was heated to 80° C. for 16 hours, then concentrated. The residue was partitioned between water and ethyl acetate, washed with saturated aqueous sodium bicarbonate solution then saturated aqueous sodium chloride solution, dried over magnesium sulfate, filtered, and concentrated. The product was purified by chromatography on a silica gel preparative thin layer chromatography plate (Eluant: 40% ethyl acetate in heptane) to give the title compound as a foam. Yield: 269 mg, 0.75 mMol, 77%. LCMS m/z 357.6 (M−1). ¹H NMR (400 MHz, CHLOROFORM-d) δ 0.72 (d, J=6.6 Hz, 3H), 1.14 (d, J=6.8 Hz, 3H), 1.83 (m, 1H), 2.33 (m, 1H), 2.49 (m, 1H), 2.80 (ddd, apparent td, J=11.0, 11.0, 4.5 Hz, 1H), 3.54 (dd, apparent t, J=11.4, 11.4 Hz, 1H), 3.61 (ddd, apparent td, J=12.2, 12.2, 1.9 Hz, 1H), 3.70 (m, 1H), 4.02 (dd, J=11.8, 4.6 Hz, 1H), 4.09 (br dd, J=11.7, 4.3 Hz, 1H), 4.75 (d, J=8.5 Hz, 1H), 7.34 (d, J=8.3 Hz, 2H), 7.37 (m, 1H), 7.46 (m, 2H), 7.57 (m, 4H).

### Examples 3, 4, 5, and 6

### N-[(3S,4R)-4-biphenyl-4-yl-4-hydroxytetrahydro-2H-pyran-3-yl]propane-2-sulfonamide, (1)

### N-[(3R,4S)-4-biphenyl-4-yl-4-hydroxytetrahydro-2H-pyran-3-yl]propane-2-sulfonamide, (4)

### N-[(3S,4S)-4-biphenyl-4-yl-4-hydroxytetrahydro-2H-pyran-3-yl]propane-2-sulfonamide, (5) and

### N-[(3R,4R)-4-biphenyl-4-yl-4-hydroxytetrahydro-2H-pyran-3-yl]propane-2-sulfonamide (6)

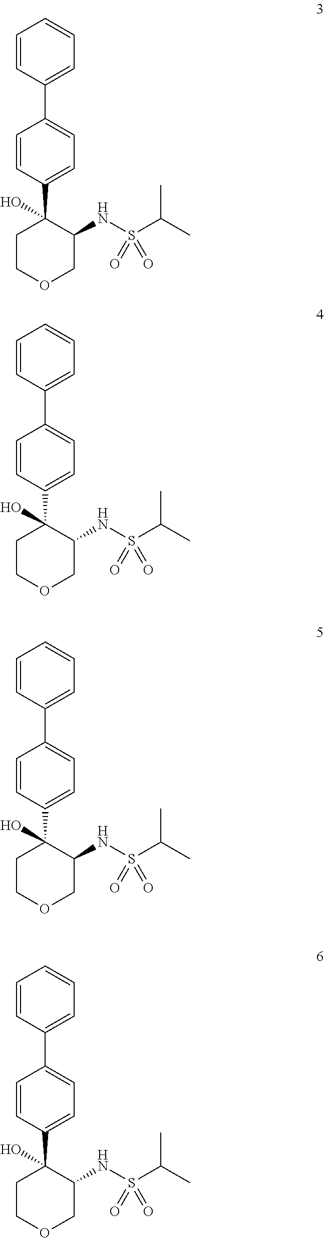

Step 1: Preparation of 3-bromotetrahydro-4H-pyran-4-one. N-Bromosuccinimide (NBS) (187 g, 1.05 mol) was added slowly to a suspension of tetrahydro-4H-pyran-4-one (100 g, 1 Mol) and ammonium acetate (7.7 g, 0.1 Mol) in diethyl ether (500 mL) at 0° C. The resulting mixture was stirred at room temperature overnight. The reaction mixture was filtered and the filtrate was concentrated. The crude product was purified by silica gel chromatography (Gradient: 20 to 40% diethyl ether in hexanes) to give 3-bromotetrahydro-4H-pyran-4-one. Yield: 130 g, 0.73 Mol, 73%. GCMS m/z 181/179 (M+1). ¹H NMR (400 MHz, CHLOROFORM-d) δ 2.59 (m, 1H), 2.90 (ddd, J=14.7, 5.4, 4.4 Hz, 1H), 3.85 (m, 2H), 4.05 (m, 1H), 4.23 (ddd, J=12.0, 5.2, 1.2 Hz, 1H), 4.44 (ddd, J=7.9, 5.2, 1.5 Hz, 1H).

Step 2: Preparation of 2-(4-oxotetrahydro-2H-pyran-3-yl)-1H-isoindole-1,3(2H)-dione. Potassium phthalimide (44.4 g, 240 mMol) was slowly added to a solution of 3-bromotetrahydro-4H-pyran-4-one (35.8 g, 200 mMol) in an anhydrous mixture of tetrahydrofuran-dimethylformamide (3:1, 600 mL) at room temperature. The reaction mixture was stirred at room temperature for 4 days. The solids were filtered off and the filtrate was concentrated. The resulting crude product was purified by silica gel chromatography (Gradient: 50 to 60% ethyl acetate in hexanes) to give 2-(4-oxotetrahydro-2H-pyran-3-yl)-1H-isoindole-1,3(2H)-dione. Yield: 25 g, 100 mMol, 50%. GCMS m/z 246 (M+1). ¹H NMR (400 MHz, CHLOROFORM-d) δ 2.62 (br d, J=15.3 Hz, 1H), 2.78 (m, 1H), 3.87 (ddd, apparent td, J=11.6, 11.6, 2.7 Hz, 1H), 4.29 (m, 3 H), 4.94 (dd, J=10.8, 7.9 Hz, 1H), 7.72 (m, 2H), 7.82 (m, 2H).

Step 3: Preparation of 2-(1,4,8-trioxaspiro[4.5]dec-6-yl)-1H-isoindole-1,3(2H)-dione. A solution of 2-(4-oxotetrahydro-2H-pyran-3-yl)-1H-isoindole-1,3(2H)-dione (24.5 g, 100 mMol), para-toluenesulfonic acid monohydrate (PTSA) (0.95 g, 5 mMol) and ethylene glycol (16.7 mL, 300 mMol) in toluene (500 mL) was heated to reflux with a Dean-Stark trap for 5 days. Upon completion of the reaction, the solution was cooled to room temperature and the toluene was removed in vacuo. The residue was partitioned between methylene chloride (500 mL) and saturated aqueous sodium bicarbonate solution, washed with saturated aqueous sodium chloride solution, dried over sodium sulfate, filtered, and concentrated. The residue was triturated with diethyl ether (100 mL) to give 2-(1,4,8-trioxaspiro[4.5]dec-6-yl)-1H-isoindole-1,3(2H)-dione as a grey solid, which was used without further purification. Yield: 24 g, 83 mMol, 83%. LCMS m/z 290.2 (M+1). ¹H NMR (400 MHz, CHLOROFORM-d) δ 1.78 (m, 1H), 1.89 (ddd, J=12.9, 12.9, 5.1 Hz, 1H), 3.66 (m, 3H), 3.86 (m, 4H), 4.41 (dd, J=11.1, 4.7 Hz, 1H), 4.68 (dd, apparent t, J=11.1, 11.1 Hz, 1H), 7.68 (m, 2H), 7.77 (m, 2H).

Step 4: Preparation of 1,4,8-trioxaspiro[4.5]decan-6-amine. Hydrazine hydrate (20.2 mL, 415 mMol) was slowly added to a suspension of 2-(1,4,8-trioxaspiro[4.5]dec-6-yl)-1H-isoindole-1,3(2H)-dione (24 g, 83 mMol) in ethanol (600 mL) at room temperature. Upon completion of the addition, the contents were refluxed for 18 hours. Heavy precipitation was observed. The reaction mixture was cooled to room temperature and the supernatant was decanted. The solids were washed with ethyl acetate (2×300 mL) and the combined supernatant and filtrates were concentrated to a residue. The residue was triturated with ethyl acetate (200 mL) to remove the remaining byproduct as a solid. The solution was concentrated to afford 1,4,8-trioxaspiro[4.5]decan-6-amine as an oil of 90% purity that was used in the next step without further purification. Yield: 11 g, 69 mMol, 83%. LCMS m/z 160.0 (M+1). ¹H NMR (400 MHz, CHLOROFORM-d) δ 1.55-1.62 (m, 1H), 1.83-1.89 (m, 1H), 2.80 (dd, J=7.9, 4.1 Hz, 1H), 3.40 (m, 1H), 3.59-3.65 (m, 1H), 3.70-3.76 (m, 1H), 3.80 (dd, J=11.2, 4.1 Hz, 1H), 3.98 (s, 4H).

Step 5: Preparation of N-(1,4,8-trioxaspiro[4.5]dec-6-yl)propane-2-sulfonamide. Triethylamine (19.3 mL, 138.4 mMol), N,N-dimethylpyridin-4-amine (DMAP) (8.4 g, 69.2 mMol) and propane-2-sulfonyl chloride (15.47 mL, 138.4 mMol) were added sequentially to a solution of 1,4,8-trioxaspiro[4.5]decan-6-amine (11 g, 69.2 mMol) in anhydrous methylene chloride (400 mL) at 0° C. The reaction mixture was slowly warmed to room temperature and stirred for 18 hours. After being quenched with saturated aqueous sodium bicarbonate solution (30 mL), the layers were separated. The organic layer was washed with saturated aqueous sodium chloride solution, dried over sodium sulfate, filtered, and concentrated. The crude product was purified by silica gel chromatography (Gradient: 40 to 70% ethyl acetate in hexanes) to give N-(1,4,8-trioxaspiro[4.5]dec-6-yl)propane-2-sulfonamide as an oil. Yield: 2.1 g, 7.6 mMol, 11%. GCMS m/z 266 (M+1). ¹H NMR (400 MHz, CHLOROFORM-d) δ 1.40 (2 overlapping doublets, 6H), 1.65-1.76 (m, 1H), 1.84-1.91 (m, 1H), 3.22-3.32 (m, 1H), 3.40-3.70 (m, 3H), 3.75-3.84 (m, 2H), 3.97-4.16 (m, 4H), 4.48 (d, J=8.8 Hz, 1H).

Step 6: Preparation of N-(4-oxotetrahydro-2H-pyran-3-yl)propane-2-sulfonamide. A solution of N-(1,4,8-trioxaspiro[4.5]dec-6-yl)propane-2-sulfonamide (2.1 g, 7.92 mmol) and para-toluenesulfonic acid monohydrate (PTSA) (3.0 g, 15.8 mMol) in acetone (80 mL) and water (34 mL) was refluxed for 3 days. GC/MS analysis of the reaction mixture showed only 5% conversion. Concentrated sulfuric acid (5 mL) was added in several portions over 3 days until complete deprotection was observed. The volatiles were removed and the residue was dissolved in ethyl acetate (300 mL) and washed five times with saturated aqueous sodium bicarbonate solution. The organic layer was concentrated and the crude residue was purified by silica gel chromatography (Gradient: 60 to 80% ethyl acetate in hexanes) to give N-(4-oxotetrahydro-2H-pyran-3-yl)propane-2-sulfonamide as an oil. Yield: 1.2 g, 5.5 mMol, 69%. GCMS m/z 222 (M+1). ¹H NMR (400 MHz, CHLOROFORM-d) δ 1.40 (d, 6H), 2.55-2.61 (m, 1H), 2.74-2.85 (m, 1H), 3.11-3.20 (m, 1H), 3.31 (dd, apparent t, J=10.6, 10.6 Hz, 1H), 3.58-3.67 (m, 1H), 4.24-4.36 (m, 2H), 4.47-4.53 (m, 1H), 5.24-5.31 (m, 1H).

Step 7: Preparation of trans-N-{4-hydroxy-4-[4-(trimethylsilyl)phenyl]tetrahydro-2H-pyran-3-yl}propane-2-sulfonamide and cis-N-{4-hydroxy-4-[4-(trimethylsilyl)phenyl]tetrahydro-2H-pyran-3-yl}propane-2-sulfonamide. [4-(Trimethylsilyl)phenyl]magnesium bromide (see M. M. Goodman et al., PCT Pat. Appl. Publ. WO9706832, 1997) (0.5M solution in tetrahydrofuran, 80 mL, 40 mMol) was added to a 0° C. solution of N-(4-oxotetrahydro-2H-pyran-3-yl)propane-2-sulfonamide (4.87 g, 22 mMol) in tetrahydrofuran (10 mL). The reaction was stirred for 4 hours at 0° C. and for 64 hours at room temperature. The reaction was cooled to 0° C. and a saturated aqueous solution of ammonium chloride was added. The reaction was extracted twice with ethyl acetate and the combined organics were dried over sodium sulfate, filtered, and concentrated in vacuo. The crude product was purified by silica gel chromatography (Gradient: 20 to 60% ethyl acetate in heptane) to give pure trans-N-{4-hydroxy-4-[4-(trimethylsilyl)phenyl]tetrahydro-2H-pyran-3-yl}propane-2-sulfonamide (0.936 g), and a mixture of trans-N-{4-hydroxy-4-[4-(trimethylsilyl)phenyl]tetrahydro-2H-pyran-3-yl}propane-2-sulfonamide and cis-N-{4-hydroxy-4-[4-(trimethylsilyl)phenyl]tetrahydro-2H-pyran-3-yl}propane-2-sulfonamide (6.0 g). This mixture was repurified by silica gel chromatography (Gradient: 20 to 60% ethyl acetate in heptane) to afford additional pure trans-N-{4-hydroxy-4-[4-(trimethylsilyl)phenyl]tetrahydro-2H-pyran-3-yl}propane-2-sulfonamide. Total yield: 2.586 g, 6.96 mMol, 32%. LCMS m/z 370.1 (M−1). ¹H NMR (400 MHz, CHLOROFORM-d) δ 0.27 (s, 9H), 0.67 (d, J=6.6 Hz, 3H), 0.97 (d, J=6.8 Hz, 3H), 1.66 (m, 2H), 1.87 (br s, 1H), 2.51 (m, 1H), 3.64 (br d, J=9.3 Hz, 1H), 3.85 (br d, J=11.6 Hz, 1H), 3.93 (m, 2H), 4.20 (br d, J=11.4 Hz, 1H), 4.69 (d, J=9.3 Hz, 1H), 7.49 (d, J=7.7 Hz, 2H), 7.56 (d, J=7.3 Hz, 2H).

Mixed fractions from chromatography were recrystallized from a 40:60 mixture of ethanol/heptane to give an approximately 4:1 mixture of cis-N-{4-hydroxy-4-[4-(trimethylsilyl)phenyl]tetrahydro-2H-pyran-3-yl}propane-2-sulfonamide and trans-N-{4-hydroxy-4-[4-(trimethylsilyl)phenyl]tetrahydro-2H-pyran-3-yl}propane-2-sulfonamide (2.0 g, 5.4 mMol, 24%). ¹H NMR (400 MHz, CHLOROFORM-d) δ selected peaks: 0.60 (major) and 0.69 (minor) (2 doublets, J=6.8, 6.6 Hz, 3H), 0.98 (minor) and 1.00 (major) (2 doublets, J=6.8, 6.8 Hz, 3H), 4.64 (major) and 4.70 (minor) (2 br doublets, J=9.1, 9.3 Hz, 1H).

Step 8: Preparation of cis-N-[4-(4-bromophenyl)-4-hydroxytetrahydro-2H-pyran-3-yl]propane-2-sulfonamide and trans-N-[4-hydroxy-4-(4-bromophenyl)tetrahydro-2H-pyran-3-yl]propane-2-sulfonamide. An approximately 4:1 mixture of cis-N-{4-hydroxy-4-[4-(trimethylsilyl)phenyl]tetrahydro-2H-pyran-3-yl}propane-2-sulfonamide and trans-N-{4-hydroxy-4-[4-(trimethylsilyl)phenyl]tetrahydro-2H-pyran-3-yl}propane-2-sulfonamide (0.50 g, 1.35 mMol), potassium bromide (0.24 g, 2.02 mMol), acetic acid (9 mL), and methanol (1.5 mL) was stirred at 60° C. for 20 minutes. N-Chlorosuccinimide (NCS) (0.216 g, 1.62 mmol) was added and the reaction continued at 60° C. for 4 hours. The reaction was allowed to cool to room temperature and poured into a mixture of sodium hydroxide (10.5 g) and ice (90 g). The aqueous solution was extracted three times with ethyl acetate and the combined organic layers were dried over sodium sulfate, filtered, and concentrated. The crude product was triturated with chloroform and the white solids collected by filtration; the filtrate was concentrated and the trituration/collection process repeated. The white solids were combined to give products cis-N-[4-(4-bromophenyl)-4-hydroxytetrahydro-2H-pyran-3-yl]propane-2-sulfonamide and trans-N-[4-hydroxy-4-(4-bromophenyl)tetrahydro-2H-pyran-3-yl]propane-2-sulfonamide in an approximately 3.6:1 ratio. Yield: 504 mg, 1.33 mMol, 99%. LCMS for cis-N-[4-(4-bromophenyl)-4-hydroxytetrahydro-2H-pyran-3-yl]propane-2-sulfonamide: m/z 378.3/376.3 (M−1). LCMS for trans-N-[4-hydroxy-4-(4-bromophenyl)tetrahydro-2H-pyran-3-yl]propane-2-sulfonamide: m/z 378.2/376.3 (M−1). ¹H NMR (400 MHz, DIMETHYLSULFOXIDE-d₆) δ Selected peaks: 0.56 (major) and 0.62 (minor) (2 doublets, J=6.6, 6.8 Hz, 3H), 0.90 (minor) and 0.93 (major) (2 doublets, J=6.8, 6.8 Hz, 3H), 1.44 (minor) and 1.57 (major) (2 br doublets, J=14.1, 13.7 Hz, 1H).

Step 9: Preparation of trans-N-[4-biphenyl-4-yl-4-hydroxytetrahydro-2H-pyran-3-yl]propane-2-sulfonamide and cis-N-[4-biphenyl-4-yl-4-hydroxytetrahydro-2H-pyran-3-yl]propane-2-sulfonamide. A mixture of cis-N-[4-(4-bromophenyl)-4-hydroxytetrahydro-2H-pyran-3-yl]propane-2-sulfonamide and trans-N-[4-hydroxy-4-(4-bromophenyl)tetrahydro-2H-pyran-3-yl]propane-2-sulfonamide in approximately a 3.6:1 ratio (100 mg, 0.26 mMol), phenylboronic acid (64 mg, 0.528 mMol), sodium carbonate (140 mg, 1.32 mMol), ethanol (3.2 mL), toluene (0.5 mL), and water (0.5 mL) was evacuated and backfilled with nitrogen three times. Tetrakis(triphenylphosphine)palladium(0) (30 mg, 0.026 mMol) was added and degassing repeated. The reaction was heated to 80° C. for 18 hours, cooled, and filtered. The filtrate was concentrated and subjected to silica gel chromatography (Gradient: 20 to 60% ethyl acetate in heptane) to remove some impurities. This partially purified material was combined with the similarly treated product of a parallel reaction (which also employed 100 mg of a mixture of cis-N-[4-(4-bromophenyl)-4-hydroxytetrahydro-2H-pyran-3-yl]propane-2-sulfonamide and trans-N-[4-hydroxy-4-(4-bromophenyl)tetrahydro-2H-pyran-3-yl]propane-2-sulfonamide, but in an approximately 1:1 ratio) and repurified by reverse phase HPLC using a Waters Xterra C₁₈ column, 30×50 mm, mobile phase A=water+0.1% trifluoroacetic acid; mobile phase B=acetonitrile+0.1% trifluoroacetic acid. Gradient: 30% B to 70% B over 8 minutes) with a flow rate of 40 mL/min. Material with a retention time of 3.98 minutes was collected to afford the racemic mixture of the trans enantiomers trans-N-[4-biphenyl-4-yl-4-hydroxytetrahydro-2H-pyran-3-yl]propane-2-sulfonamide (52 mg, 0.139 mMol, 26%). Material with a retention time of 4.42 minutes was collected to afford the racemic mixture of the cis enantiomers cis-N-[4-biphenyl-4-yl-4-hydroxytetrahydro-2H-pyran-3-yl]propane-2-sulfonamide (53.3 mg, 0.142 mMol, 27%).

Step 10: Isolation of N-[(3S,4R)-4-biphenyl-4-yl-4-hydroxytetrahydro-2H-pyran-3-yl]propane-2-sulfonamide and N-[(3R,4S)-4-biphenyl-4-yl-4-hydroxytetrahydro-2H-pyran-3-yl]propane-2-sulfonamide. The trans enantiomers trans-N-[4-biphenyl-4-yl-4-hydroxytetrahydro-2H-pyran-3-yl]propane-2-sulfonamide (52 mg, 0.139 mMol) were subjected to chiral HPLC chromatography using a Diacel OJ-H column, 5 μm, 10 mm×250 mm, mobile phase: 70/30 mixture of carbon dioxide/propanol at a flow rate of 10 mL/minute.

Material with a retention time of 3.46 minutes was collected to yield N-[(3S,4R)-4-biphenyl-4-yl-4-hydroxytetrahydro-2H-pyran-3-yl]propane-2-sulfonamide. Yield: 16 mg, 0.042 mMol, 30%. LCMS m/z 374.1 (M−1). ¹H NMR (400 MHz, METHANOL-d₄) δ 0.71 (d, J=6.6 Hz, 3H), 0.96 (d, J=6.8 Hz, 3H), 1.68 (br d, J=13.8 Hz, 1H), 2.12 (m, 1H), 2.76 (m, 1H), 3.49 (br s, 1H), 3.79 (br d, J=11.0 Hz, 1H), 3.95 (m, 2H), 4.22 (dd, J=11.4, 1.7 Hz, 1H), 7.34 (br t, J=7.4, Hz, 1H), 7.44 (dd, apparent t, J=7.7, 7.7 Hz, 2H), 7.60 (m, 2H), 7.63 (s, 4H). The absolute configuration of this material was established via X-ray crystallography.

Material with a retention time of 4.41 minutes was collected to yield N-[(3R,4S)-4-biphenyl-4-yl-4-hydroxytetrahydro-2H-pyran-3-yl]propane-2-sulfonamide. Yield: 19 mg, 0.05 mMol, 34%. LCMS m/z 374.1 (M−1). ¹H NMR (400 MHz, METHANOL-d₄) δ 0.71 (d, J=6.6 Hz, 3H), 0.96 (d, J=6.8 Hz, 3H), 1.68 (br d, J=13.9 Hz, 1H), 2.12 (m, 1H), 2.75 (m, 1H), 3.49 (br s, 1H), 3.79 (br d, J=11.2 Hz, 1H), 3.95 (m, 2H), 4.22 (dd, J=11.4, 1.5 Hz, 1H), 7.34 (br t, J=7.4 Hz, 1H), 7.43 (dd, apparent t, J=7.6, 7.6 Hz, 2H), 7.61 (m, 2H), 7.63 (s, 4H).

Step 11: Isolation of N-[(3S,4S)-4-biphenyl-4-yl-4-hydroxytetrahydro-2H-pyran-3-yl]propane-2-sulfonamide and N-[(3R,4R)-4-biphenyl-4-yl-4-hydroxytetrahydro-2H-pyran-3-yl]propane-2-sulfonamide The cis enantiomers cis-N-[4-biphenyl-4-yl-4-hydroxytetrahydro-2H-pyran-3-yl]propane-2-sulfonamide (53.3 mg, 0.142 mMol) were subjected to chiral HPLC chromatography using a Diacel AS-H column, 5 μm, 10 mm×250 mm, mobile phase: 85/15 carbon dioxide/methanol at a flow rate of 10 mL/minute.

Material with a retention time of 5.21 minutes was collected to yield N-[(3S,4S)-4-biphenyl-4-yl-4-hydroxytetrahydro-2H-pyran-3-yl]propane-2-sulfonamide. Yield: 24 mg, 0.063 mMol, 44%. LCMS m/z 374 (M−1). ¹H NMR (400 MHz, METHANOL-d₄) δ 0.64 (d, J=6.6 Hz, 3H), 0.96 (d, J=6.8 Hz, 3H), 1.74 (br d, J=14.3 Hz, 1H), 2.23 (m, 1H), 2.38 (m, 1H), 3.70 (m, 2H), 3.80 (m, 1H), 3.90 (m, 2H), 7.34 (br t, J=7.4 Hz, 1H), 7.44 (dd, J=7.6, 7.6 Hz, 2 H), 7.61 (m, 2H), 7.63 (d, J=8.7 Hz, 2H), 7.68 (d, J=8.7 Hz, 2H). The absolute configuration of this material was established via X-ray crystallography.

Material with a retention time of 7.17 minutes was collected to yield N-[(3R,4R)-4-biphenyl-4-yl-4-hydroxytetrahydro-2H-pyran-3-yl]propane-2-sulfonamide. Yield: 24 mg, 0.063 mMol, 44%. LCMS m/z 374.1 (M−1). ¹H NMR (400 MHz, METHANOL-d₄) δ 0.64 (d, J=6.8 Hz, 3H), 0.96 (d, J=6.8 Hz, 3H), 1.74 (br d, J=14.3 Hz, 1H), 2.24 (m, 1H), 2.38 (m, 1H), 3.71 (m, 2H), 3.80 (m, 1H), 3.90 (m, 2H), 7.34 (br t, J=7.4 Hz, 1H), 7.44 (dd, J=7.7, 7.7 Hz, 2H), 7.61 (m, 2H), 7.63 (d, J=8.5 Hz, 2H), 7.68 (d, J=8.7 Hz, 2H).

### Examples 7 and 8

### Preparation of N-[(3S,4S)-4-(2′-ethoxybiphenyl-4-yl)tetrahydro-2H-pyran-3-yl]propane-2-sulfonamide and N-[(3R,4R)-4-(2′-ethoxybiphenyl-4-yl)tetrahydro-2H-pyran-3-yl]propane-2-sulfonamide

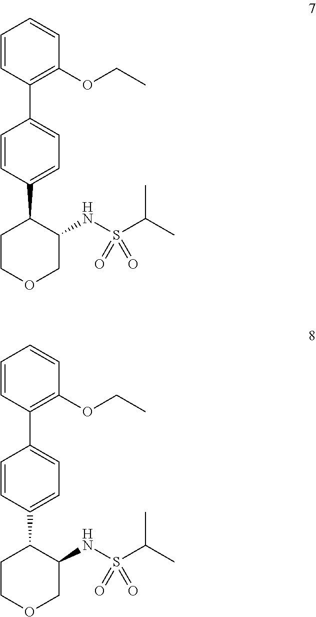

Step 1: Preparation of ethyl 4-{[(trifluoromethyl)sulfonyl]oxy}-3,6-dihydro-2H-pyran-3-carboxylate. A solution of ethyl 4-oxotetrahydro-2H-pyran-3-carboxylate (see Jiao et al., U.S. Pat. Appl. Publ. 20050107422, 2005) (1.0 g, 5.8 mMol) in tetrahydrofuran (5 mL) was added to a slurry of sodium hydride (60% in mineral oil, 302 mg, 7.6 mMol) and the mixture was stirred for 2 hours at room temperature. The reaction was cooled to −78° C. and N-phenyl-bis(trifluoromethanesulfonimide) (2.28 g, 6.4 mMol) was added. The reaction was allowed to warm to room temperature and stir for 16 hours. The reaction mixture was quenched by the addition of saturated aqueous sodium bicarbonate solution (30 mL) and the layers were separated. The aqueous layer was washed with diethyl ether and the combined organic layers dried over sodium sulfate, filtered, and concentrated to give ethyl 4-{[(trifluoromethyl)sulfonyl]oxy}-3,6-dihydro-2H-pyran-3-carboxylate, which was generally used in the next step without further purification. Yield: 1.97 g, assumed quantitative. ¹H NMR (400 MHz, CHLOROFORM-d) δ 1.32 (t, J=7.2 Hz, 3H), 2.52 (m, 2H), 3.88 (t, J=5.5 Hz, 2H), 4.28 (q, J=7.1 Hz, 2H), 4.44 (t, J=2.8 Hz, 2H).

Step 2: Preparation of ethyl 4-[4-(trimethylsilyl)phenyl]-5,6-dihydro-2H-pyran-3-carboxylate. A flask containing ethyl 4-{[(trifluoromethyl)sulfonyl]oxy}-3,6-dihydro-2H-pyran-3-carboxylate isolated from the previous step (1.98 g, 5.8 mMol), (trimethylsilyl)phenylboronic acid (1.26 g, 6.51 mMol), tetrakis(triphenylphosphine)palladium(0) (0.752 g, 0.65 mMol), potassium carbonate (2.88 g, 20.8 mMol), and tetrahydrofuran (30 mL) was heated to 65° C. for 16 hours. The reaction was concentrated and the crude product was purified by silica gel chromatography (Gradient: 10 to 50% ethyl acetate in heptane) to give ethyl 4-[4-(trimethylsilyl)phenyl]-5,6-dihydro-2H-pyran-3-carboxylate in an approximately 2:1 molar ratio with materials from the boronic acid reagent, as an oil. Yield: 1.32 g, <4.34 mMol, <67%. LCMS m/z 305.0 (M+1). ¹H NMR (400 MHz, CHLOROFORM-d), product peaks only, δ 0.28 (s, 9H), 0.90 (t, J=7.2 Hz, 3H), 2.54 (m, 2H), 3.95 (m, 4H), 4.50 (t, J=2.6 Hz, 2H), 7.16 (d, J=8.1 Hz, 2H), 7.51 (d, J=7.9 Hz, 2H).

Step 3: Preparation of ethyl cis-4-[4-(trimethylsilyl)phenyl]tetrahydro-2H-pyran-3-carboxylate. A Parr bottle was charged with ethyl 4-[4-(trimethylsilyl)phenyl]-5,6-dihydro-2H-pyran-3-carboxylate (5.5 g, <18.06 mMol, contaminated in equimolar portion with (trimethylsilyl)phenyl material from earlier step), 10% wt/wt palladium on activated carbon catalyst (384 mg), ethyl acetate (15 mL), and absolute ethanol (90 mL), and shaken under an atmosphere of hydrogen (50 psi) for 48 hours. The catalyst was removed by filtration and the filtrate concentrated to yield crude ethyl cis-4-[4-(trimethylsilyl)phenyl]tetrahydro-2H-pyran-3-carboxylate as an oil that contained an approximately equimolar amount of material derived from (trimethylsilyl)phenylboronic acid. Yield: 4.9 g, <15.99 mMol, <88%. LCMS m/z 307.2 (M+1). ¹H NMR (400 MHz, CHLOROFORM-d), product peaks only, δ 0.29 (s, 9H), 1.02 (t, J=7.1 Hz, 3H), 1.78 (br d, J=13.1 Hz, 1H), 2.77 (m, 1H), 2.95 (br s, 1H), 3.14 (ddd, apparent dt, J=12.2, 4.1 Hz, 4.1 Hz, 1 H), 3.62 (ddd, apparent td, J=11.5, 11.5, 2.1 Hz, 1H), 3.82 (m, 1H), 3.97 (m, 2H), 4.26 (br d, J=13.1 Hz, 1H), 4.36 (d, J=11.4 Hz, 1H), 7.29 (d, J=7.9 Hz, 2H), 7.50 (d, J=8.1 Hz, 2H).

Step 4: Preparation of ethyl trans-4-[4-(trimethylsilyl)phenyl]tetrahydro-2H-pyran-3-carboxylate. A flask was charged with the ester ethyl cis-4-[4-(trimethylsilyl)phenyl]tetrahydro-2H-pyran-3-carboxylate (4.8 g, 15.66 mMol), ethanol (60 mL), and 21 weight % sodium ethoxide in ethanol (6.43 mL, 17.2 mMol) in that order and the mixture refluxed for 16 hours. The reaction was concentrated, the residue was neutralized with saturated aqueous ammonium chloride solution and extracted three times with methylene chloride. The combined organics were dried, filtered, and concentrated to afford ethyl trans-4-[4-(trimethylsilyl)phenyl]tetrahydro-2H-pyran-3-carboxylate as an oil that contained an approximately equimolar amount of material derived from (trimethylsilyl)phenylboronic acid. Yield: 4.4 g, <14.4 mMol, <92%. LCMS m/z 307.1 (M+1). ¹H NMR (400 MHz, CHLOROFORM-d), product peaks only, δ 0.29 (s, 9H), 0.96 (t, J=7.1 Hz, 3H), 1.82 (br d, J=11.8 Hz, 1H), 1.91 (m, 1 H), 2.98 (m, 1H), 3.06 (m, 1H), 3.63 (m, 2H), 3.95 (m, 2H), 4.14 (m, 1H), 4.25 (dd, J=11.2, 3.9 Hz, 1H), 7.24 (d, J=7.9 Hz, 2H), 7.49 (d, J=7.7 Hz, 2 H).

Step 5: Preparation of trans-4-[4-(trimethylsilyl)phenyl]tetrahydro-2H-pyran-3-carboxylic acid. The ester ethyl trans-4-[4-(trimethylsilyl)phenyl]tetrahydro-2H-pyran-3-carboxylate (4.4 g, 14.4 mMol), aqueous sodium hydroxide (2N, 63.4 mMol, 31.7 mL), and methanol (75 mL) were combined and refluxed for 4 hours. The reaction was concentrated and the residue partitioned between water and diethyl ether. The aqueous phase was brought to pH 1 with concentrated hydrochloric acid and extracted three times with methylene chloride. The organics were dried, filtered, and concentrated to afford trans-4-[4-(trimethylsilyl)phenyl]tetrahydro-2H-pyran-3-carboxylic acid as a green solid of sufficient purity to use in the next reaction. Yield: 3.0 g, 10.8 mMol, 75%. LCMS m/z 277.1 (M−1). ¹H NMR (400 MHz, CHLOROFORM-d) δ 0.32 (s, 9H), 1.85 (m, 2H), 3.07 (m, 2H), 3.60 (m, 2H), 4.12 (br d, J=11.5 Hz, 1H), 4.31 (dd, J=11.1, 3.2 Hz, 1H), 7.25 (d, J=7.9 Hz, 2H), 7.52 (d, J=7.9 Hz, 2H).

Step 6: Preparation of benzyl trans-{4-[4-(trimethylsilyl)phenyl]tetrahydro-2H-pyran-3-yl}carbamate To a solution of trans-4-[4-(trimethylsilyl)phenyl]tetrahydro-2H-pyran-3-carboxylic acid (2.7 g, 9.7 mMol) in anhydrous dichloroethane (20 mL) was added triethylamine (2.3 mL, 16.5 mMol) and diphenylphosphoryl azide (DPPA) (3.14 mL, 14.5 mMol). The reaction was stirred at room temperature for 20 minutes then refluxed for 2.5 hours. Benzyl alcohol (1.71 mL, 16.6 mMol) was added and the reaction refluxed for 20 hours. The reaction was concentrated and some solid removed by filtration. The filtrate was purified by silica gel chromatography (Gradient: 0 to 15% ethyl acetate in heptane) to provide benzyl trans-{4-[4-(trimethylsilyl)phenyl]tetrahydro-2H-pyran-3-yl}carbamate as an oil containing residual benzyl alcohol and materials derived from (trimethylsilyl)phenylboronic acid. Yield: 1.45 g, <3.78 mMol, <39%. LCMS m/z 384.5 (M+1). ¹H NMR (400 MHz, METHANOL-d₄) δ Selected peaks: 0.24 (s, 9H), 1.82 (m, 2H), 2.72 (ddd, apparent td, J=11.5, 11.5, 4.5 Hz, 1H), 3.18 (dd, J=10.7, 10.7 Hz, 1H), 3.45 (ddd, apparent td, J=11.5, 11.5, 2.5 Hz, 1H).

Step 7: Preparation of trans-4-[4-(trimethylsilyl)phenyl]tetrahydro-2H-pyran-3-amine. A Parr bottle was charged with a mixture of benzyl trans-{4-[4-(trimethylsilyl)phenyl]tetrahydro-2H-pyran-3-yl}carbamate (3.5 g, 9.1 mMol), absolute ethanol (140 mL), acetic acid (1.57 mL), and 10% wt/wt palladium on activated carbon catalyst (380 mg) and shaken under an atmosphere of hydrogen (45 psi) for 16 hours. The catalyst was removed by filtration and the filtrate concentrated. The residue was dissolved in methylene chloride (140 mL) and neutralized with aqueous sodium hydroxide (1N, 140 mL). The phases were separated and the aqueous solution extracted twice with methylene chloride. The organics were dried over sodium sulfate, filtered, and concentrated to give trans-4-[4-(trimethylsilyl)phenyl]tetrahydro-2H-pyran-3-amine as an oil containing residual benzyl alcohol. Yield: 2.3 g, 9.1 mMol, essentially quantitative. LCMS m/z 250.2 (M+1). ¹H NMR (400 MHz, METHANOL-d₄) δ Selected peaks: 0.25 (s, 9H), 1.72 (m, 1H), 1.87 (m, 1H), 2.47 (ddd, J=12.1, 10.4, 4.0 Hz, 1H), 3.15 (dd, apparent t, J=10.7, 10.7 Hz, 1 H), 3.50 (ddd, apparent td, J=11.9, 11.9, 2.2 Hz, 1H), 4.50 (m, 1H), 7.51 (d, J=8.1 Hz, 2H).

Step 8: Preparation of trans-N-{4-[4-(trimethylsilyl)phenyl]tetrahydro-2H-pyran-3-yl}propane-2-sulfonamide. A flask was charged with a mixture of trans-4-[4-(trimethylsilyl)phenyl]tetrahydro-2H-pyran-3-amine (2.1 g, 8.4 mMol), 1,8-diazabicyclo[5.4.0]undec-7-ene (DBU) (1.76 mL, 11.8 mMol) and methylene chloride (10 mL), and placed in a 0° C. cooling bath. Propane-2-sulfonyl chloride (1.07 mL, 9.26 mMol) was added drop-wise and the reaction was allowed to warm to room temperature and stir for 16 hours. The reaction was concentrated and partitioned between ethyl acetate and water. The organics were dried and concentrated to an oil, which was purified by silica gel chromatography (Gradient: 5 to 90% ethyl acetate in heptane) to provide trans-N-{4-[4-(trimethylsilyl)phenyl]tetrahydro-2H-pyran-3-yl}propane-2-sulfonamide as a white solid. Yield: 1.7 g, 4.78 mMol, 57%. LCMS m/z 356.0 (M+1). ¹H NMR (400 MHz, CHLOROFORM-d) δ 0.26 (s, 9H), 0.56 (d, J=6.6 Hz, 3H), 1.09 (d, J=7.1 Hz, 3H), 1.88 (m, 1H), 2.03 (m, 1H), 2.49 (m, 2H), 3.19 (dd, apparent t, J=10.7, 10.7 Hz, 1H), 3.49 (m, 2H), 3.86 (d, J=7.7 Hz, 1 H), 4.04 (dd, J=11.5, 3.8 Hz, 1H), 4.38 (dd, J=11.2, 4.8 Hz, 1H), 7.26 (d, J=7.9 Hz, 2H), 7.51 (d, J=7.9 Hz, 2H).

Step 9: Preparation of trans-N-[4-(4-bromophenyl)tetrahydro-2H-pyran-3-yl]propane-2-sulfonamide. Trans-N-{4-[4-(trimethylsilyl)phenyl]tetrahydro-2H-pyran-3-yl}propane-2-sulfonamide (0.72 g, 2.02 mMol), potassium bromide (0.361 g, 3.04 mMol), acetic acid (13 mL) and methanol (2.0 mL) were stirred together at 60° C. for 20 minutes. N-Chlorosuccinimide (NCS) (0.324 g, 2.43 mMol) was added and the reaction continued at 60° C. for 4 hours. The reaction was allowed to cool to room temperature and poured into a mixture of sodium hydroxide (16 g) and ice (130 g). The aqueous solution was extracted three times with ethyl acetate and the combined organic layers dried over sodium sulfate, filtered, and concentrated. The crude product was triturated with 10% chloroform/heptane and the white solids collected by filtration to give trans-N-[4-(4-bromophenyl)tetrahydro-2H-pyran-3-yl]propane-2-sulfonamide. Yield: 700 mg, 1.93 mMol, 96%. LCMS m/z 360.0/362.0 (M−1). ¹H NMR (400 MHz, CHLOROFORM-d) δ 0.76 (d, J=6.6 Hz, 3H), 1.14 (d, J=6.8 Hz, 3H), 1.86 (m, 1H), 1.96 (m, 1H), 2.51 (ddd, J=11.4, 11.4, 4.4 Hz, 1H), 2.60 (m, 1 H), 3.18 (dd, J=11.0, 10.4 Hz, 1H), 3.45 (ddd, apparent td, J=11.7, 11.7, 2.4 Hz, 1H), 3.51 (m, 1H), 3.84 (d, J=8.5 Hz, 1H), 4.03 (dd, J=10.6, 3.3 Hz, 1 H), 4.36 (dd, J=11.2, 4.8 Hz, 1H), 7.16 (d, J=8.3 Hz, 2H), 7.50 (d, J=8.3 Hz, 2H).

Step 10: Preparation of N-[(3S,4S)-4-(2′-ethoxybiphenyl-4-yl)tetrahydro-2H-pyran-3-yl]propane-2-sulfonamide and N-[(3R,4R)-4-(2′-ethoxybiphenyl-4-yl)tetrahydro-2H-pyran-3-yl]propane-2-sulfonamide. A vial charged with trans-N-[4-(4-bromophenyl)tetrahydro-2H-pyran-3-yl]propane-2-sulfonamide (150 mg, 0.41 mMol), 2-ethoxyphenyl)boronic acid (103 mg, 0.621 mMol), 2-dicyclohexylphosphino-2′,4′,6′-thisopropyl-1,1′-biphenyl (X-phos) (19.5 mg, 0.041 mMol), palladium(II) acetate (6.1 mg, 0.027 mMol), potassium fluoride (120 mg, 2.07 mMol), methanol (1.5 mL), and toluene (1.5 mL) was evacuated under vacuum then filled with nitrogen three times. The reaction was heated to 130° C. in a microwave reactor for 35 minutes. The reaction was concentrated and partitioned between methylene chloride and saturated aqueous sodium bicarbonate solution, the aqueous phase was extracted twice with methylene chloride, and the combined organics were dried over sodium sulfate, filtered, and concentrated. The crude racemate was purified by silica gel chromatography (Gradient: 10 to 70% ethyl acetate in heptane) to give racemic product. Yield: 160 mg, 0.386 mMol, 94%. This material was then subjected to chiral HPLC chromatography using a Chiralpak AD-H column, 5 m, 2.1 cm×25 cm, mobile phase: 70/30 mixture of carbon dioxide/ethanol at a flow rate of 65 g/minute.

Material with a retention time of 2.36 minutes was collected to yield N-[(3S,4S)-4-(2′-ethoxybiphenyl-4-yl)tetrahydro-2H-pyran-3-yl]propane-2-sulfonamide. Yield: 72 mg, 0.18 mMol, 42%. LCMS m/z 402.1 (M−1). ¹H NMR (400 MHz, CHLOROFORM-d) δ 0.62 (d, J=6.6 Hz, 3H), 1.12 (d, J=6.8 Hz, 3H), 1.35 (t, J=6.9 Hz, 3H), 1.92 (m, 1H), 2.06 (m, 1H), 2.57 (m, 2H), 3.23 (dd, apparent t, J=10.7, 10.7 Hz, 1H), 3.51 (m, 2H), 4.06 (m, 3H), 4.18 (d, J=7.9 Hz, 1H), 4.41 (dd, J=11.2, 4.8 Hz, 1H), 6.98 (br d, J=8.3 Hz, 1H), 7.02 (ddd, apparent td, J=7.5, 7.5, 1.0 Hz, 1H), 7.31 (m, 4H), 7.56 (d, J=8.3 Hz, 2H).

Material with a retention time of 4.63 minutes was collected to yield N-[(3R,4R)-4-(2′-ethoxybiphenyl-4-yl)tetrahydro-2H-pyran-3-yl]propane-2-sulfonamide. Yield: 71 mg, 0.18 mMol, 42%. LCMS m/z 402.1 (M−1). ¹H NMR (400 MHz, CHLOROFORM-d) δ 0.62 (d, J=6.6 Hz, 3H), 1.12 (d, J=6.8 Hz, 3H), 1.35 (t, J=6.9 Hz, 3H), 1.92 (m, 1H), 2.06 (m, 1H), 2.57 (m, 2H), 3.23 (dd, apparent t, J=10.7, 10.7 Hz, 1H), 3.50 (m, 2H), 4.06 (m, 3H), 4.20 (d, J=7.9 Hz, 1H), 4.41 (dd, J=11.1, 4.7 Hz, 1H), 6.98 (br d, J=8.3 Hz, 1H), 7.02 (ddd, apparent, td, J=7.5, 7.5, 0.8 Hz, 1H), 7.31 (m, 4H), 7.56 (d, J=8.3 Hz, 2H). The absolute configuration of this material was established via X-ray crystallography.

### Example 9

### N-[(2R,3S)-2-biphenyl-4-yltetrahydro-2H-pyran-3-yl]propane-2-sulfonamide

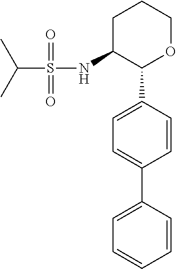

Step 1: Preparation of ethyl trans-2-(4-bromophenyl)tetrahydro-2H-pyran-3-carboxylate. To a solution of ethyl 5-bromopentanoate (24.52 mL, 164.6 mMol) and 4-bromobenzaldehyde (38.1 g, 206 mMol) in tetrahydrofuran (400 mL) at −25° C. was added drop-wise a solution of potassium tert-butoxide in tetrahydrofuran (1M, 346 mL, 346 mMol) while the internal temperature was maintained at −25° C. The solution was stirred for 1 hour at −25° C., then saturated ammonium chloride solution (200 mL) was added and the mixture was allowed to warm to room temperature and stirred for 20 minutes. The reaction was extracted three times with ethyl acetate. The combined organics were washed with saturated aqueous sodium chloride solution, dried over magnesium sulfate, filtered, and concentrated. The crude product was purified by silica gel chromatography (Eluant: 10% ethyl acetate in heptane) to provide ethyl trans-2-(4-bromophenyl)tetrahydro-2H-pyran-3-carboxylate as a light yellow crystalline solid. Yield: 42.0 g, 134 mMol, 81%. ¹H NMR (400 MHz, CHLOROFORM-d) δ 1.02 (t, J=7.2 Hz, 3H), 1.74 (m, 2H), 1.90 (m, 1 H), 2.13 (m, 1H), 2.60 (m, 1H), 3.63 (ddd, apparent td, J=11.6, 11.6, 2.9 Hz, 1H), 3.92 (m, 2H), 4.11 (m, 1H), 4.41 (d, J=10.0 Hz, 1H), 7.22 (d, J=8.5 Hz, 2H), 7.44 (d, J=8.5 Hz, 2H).

Step 2: Preparation of trans-2-(4-bromophenyl)tetrahydro-2H-pyran-3-carboxylic acid To a solution of ethyl trans-2-(4-bromophenyl)tetrahydro-2H-pyran-3-carboxylate (17.0 g, 54.3 mMol) in absolute ethanol (323 mL) was added potassium hydroxide (4.57 g, 81.4 mMol) and water (17 mL) and the mixture was refluxed for 16 hours. The reaction was concentrated in vacuo, diluted with aqueous hydrochloric acid (1N, 200 mL), and extracted three times with diethyl ether. The combined organics were washed with saturated aqueous sodium chloride solution, dried over magnesium sulfate, filtered, and concentrated to produce trans-2-(4-bromophenyl)tetrahydro-2H-pyran-3-carboxylic acid as a yellow solid of sufficient purity to use in the next reaction. Yield: 15.5 g, quantitative. ¹H NMR (400 MHz, CHLOROFORM-d) δ 1.81 (m, 3H), 2.21 (m, 1H), 2.62 (m, 1H), 3.61 (ddd, apparent td, J=11.4, 11.4, 2.9 Hz, 1H), 4.12 (br d, 1H), 4.39 (d, J=10.2 Hz, 1H), 7.22 (d, J=8.5 Hz, 2H), 7.45 (d, J=8.3 Hz, 2H).

Step 3: Preparation of trans-2-(4-bromophenyl)tetrahydro-2H-pyran-3-amine. To a solution of trans-2-(4-bromophenyl)tetrahydro-2H-pyran-3-carboxylic acid (15.5 g, 54.4 mMol) in anhydrous toluene (270 mL) was added diisopropylethylamine (9.47 mL, 54.4 mMol) and diphenylphosphoryl azide (DPPA) (22.4 g, 81.5 mMol). The reaction was stirred at 85° C. for two hours then allowed to cool to room temperature. Tetrahydrofuran (270 mL) and aqueous sodium hydroxide (2N, 135 mL) were added and the reaction was stirred for 16 hours. The reaction was diluted with water (500 mL) and extracted 3 times with ethyl acetate. The combined organics were washed with saturated aqueous sodium chloride solution, dried over sodium sulfate, filtered, and concentrated to an oil. The product was purified by silica gel chromatography (Eluant: 5% methanol in methylene chloride) to provide trans-2-(4-bromophenyl)tetrahydro-2H-pyran-3-amine as a solid. Yield: 10.2 g, 39.8 mMol, 73%. ¹H NMR (400 MHz, CHLOROFORM-d) δ 1.42 (m, 1H), 1.72 (m, 1H), 1.86 (m, 1H), 2.10 (m, 1H), 2.74 (m, 1H), 3.51 (m, 1H), 3.80 (d, J=9.1 Hz, 1H), 4.07 (m, 1H), 7.27 (d, J=8.5 Hz, 2H), 7.49 (d, J=8.5 Hz, 2 H).

Step 4: Preparation of trans-N-[2-(4-bromophenyl)tetrahydro-2H-pyran-3-yl]propane-2-sulfonamide. A flask was charged with a mixture of trans-2-(4-bromophenyl)tetrahydro-2H-pyran-3-amine (10.0 g, 39 mMol), 1,8-diazabicyclo[5.4.0]undec-7-ene (DBU) (11.9 mL, 78.1 mMol), and methylene chloride (100 mL), and placed in a 0° C. cooling bath. Propane-2-sulfonyl chloride (9.0 mL, 78.1 mMol) was added drop-wise and the reaction was allowed to warm slowly to room temperature and stir for 16 hours. The reaction was diluted with water (300 mL), the phases were separated, and the aqueous extracted with methylene chloride. The combined organics were washed with saturated aqueous sodium chloride solution, dried over magnesium sulfate, filtered, and concentrated to a solid. The product was purified by silica gel chromatography (Eluant: 5% methanol in methylene chloride) to provide trans-N-[2-(4-bromophenyl)tetrahydro-2H-pyran-3-yl]propane-2-sulfonamide as a solid. Yield: 9.0 g, 24.8 mMol, 64%. ¹H NMR (400 MHz, CHLOROFORM-d) δ 0.78 (d, J=6.6 Hz, 3H), 1.08 (d, J=6.8 Hz, 3 H), 1.60 (m, 1H), 1.75 (m, 1H), 1.88 (m, 1H), 2.33 (m, 1H), 2.44 (m, 1H), 3.32 (m, 1H), 3.49 (m, 1H), 3.94 (d, J=9.7 Hz, 1H), 4.04 (m, 1H), 4.40 (d, J=9.5 Hz, 1H), 7.27 (d, J=8.3 Hz, 2H), 7.49 (d, J=8.5 Hz, 2H).

Step 5: Isolation of N-[(2S,3R)-2-(4-bromophenyl)tetrahydro-2H-pyran-3-yl]propane-2-sulfonamide and N-[(2R,3S)-2-(4-bromophenyl)tetrahydro-2H-pyran-3-yl]propane-2-sulfonamide. The racemic material trans-N-[2-(4-bromophenyl)tetrahydro-2H-pyran-3-yl]propane-2-sulfonamide (2.2 g, 6.07 mMol) was subjected to chiral HPLC chromatography using a Chiralpak AD-H column, 5 μm, 2.1 cm×25 cm, mobile phase: 70/30 carbon dioxide/ethanol at a flow rate of 65 g/minute.

Material with a retention time of 2.62 minutes was collected to yield N-[(2S,3R)-2-(4-bromophenyl)tetrahydro-2H-pyran-3-yl]propane-2-sulfonamide. Yield: 970 mg, 2.67 mMol, 44%. MS (APCI) m/z 361.6 (M−1). ¹H NMR (400 MHz, CHLOROFORM-d) δ 0.80 (d, J=6.6 Hz, 3H), 1.09 (d, J=6.8 Hz, 3H), 1.60 (m, 1H), 1.77 (m, 1H), 1.90 (m, 1H), 2.35 (m, 1H), 2.49 (m, 1H), 3.36 (m, 1H), 3.50 (ddd, apparent td, J=11.8, 11.8, 2.3 Hz, 1H), 3.92 (d, J=9.3 Hz, 1H), 3.95 (d, J=9.7 Hz, 1H), 4.06 (m, 1H), 7.29 (d, J=7.9 Hz, 2H), 7.51 (d, J=8.3 Hz, 2H).

Material with a retention time of 3.70 minutes was collected to yield N-[(2R,3S)-2-(4-bromophenyl)tetrahydro-2H-pyran-3-yl]propane-2-sulfonamide. Yield: 900 mg, 2.5 mMol, 41%. LCMS m/z 361.6 (M−1). ¹H NMR (400 MHz, CHLOROFORM-d) δ 0.80 (d, J=6.6 Hz, 3H), 1.10 (d, J=7.1 Hz, 3H), 1.61 (m, 1H), 1.77 (m, 1H), 1.90 (m, 1H), 2.36 (m, 1H), 2.49 (m, 1H), 3.36 (m, 1H), 3.50 (ddd, apparent td, J=11.8, 11.8, 2.5 Hz, 1H), 3.88 (d, J=9.3 Hz, 1H), 3.95 (d, J=9.5 Hz, 1H), 4.06 (m, 1H), 7.28 (d, J=8.1 Hz, 2H), 7.51 (d, J=8.3 Hz, 2H). The absolute configuration of this material was established via X-ray crystallography.

Step 6: Preparation of N-[(2R,3S)-2-biphenyl-4-yltetrahydro-2H-pyran-3-yl]propane-2-sulfonamide. The title compound was prepared in a manner analogous to the preparation of Example 2, except that N-[(2R,3S)-2-(4-bromophenyl)tetrahydro-2H-pyran-3-yl]propane-2-sulfonamide was used as the starting material. Yield: 195 mg, 0.54 mMol, 49%. MS (APCI) m/z 357.6 (M−1). ¹H NMR (500 MHz, CHLOROFORM-d) δ 0.74 (d, J=6.6 Hz, 3H), 1.06 (d, J=6.8 Hz, 3H), 1.63 (m, 1H), 1.79 (br d, J=13.9 Hz, 1H), 1.94 (m, 1H), 2.32 (m, 1H), 2.54 (m, 1H), 3.47 (m, 1H), 3.54 (dd, apparent td, J=11.9, 2.3 Hz, 1H), 3.84 (d, J=9.0 Hz, 1H), 4.03 (d, J=9.5 Hz, 1H), 4.10 (m, 1H), 7.37 (m, 1H), 7.46 (m, 4H), 7.57 (m, 2H), 7.61 (d, J=8.3 Hz, 2H).

### Example 10

### N-[(2S,3R)-2-(2′-ethoxybiphenyl-4-yl)-6-oxopiperidin-3-yl]propane-2-sulfonamide

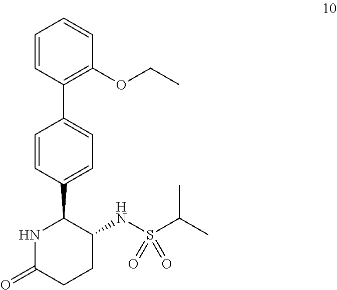

Step 1: Preparation of trans-6-(4-bromophenyl)-5-nitropiperidin-2-one. A flask was charged with 4-bromobenzaldehyde (25.0 g, 135.1 mMol), methyl 4-nitrobutanoate (23.9 g, 162 mMol), ammonium acetate (20.8 g, 270 mMol), and absolute ethanol (400 mL) and the mixture was refluxed for 16 hours. The reaction was cooled to room temperature and the solids were collected by filtration and rinsed with diethyl ether to provide trans-6-(4-bromophenyl)-5-nitropiperidin-2-one. Yield: 36.67 g, 123 mMol, 91%. LCMS m/z 299/301 (M+1). ¹H NMR (400 MHz, METHANOL-d₄) δ 2.28 (m, 1H), 2.55 (m, 3H), 5.01 (m, 1H), 5.24 (d, J=5.8 Hz, 1H), 7.33 (d, J=8.5 Hz, 2H), 7.59 (d, J=8.5 Hz, 2H).

Step 2: Preparation of trans-5-amino-6-(4-bromophenyl)piperidin-2-one A flask was charged with trans-6-(4-bromophenyl)-5-nitropiperidin-2-one (15.0 g, 50.15 mMol), tetrahydrofuran (700 mL), methanol (150 mL), and water (21 mL). Aluminum foil pieces (13.5 g, 501 mMol) were washed sequentially with diethyl ether, 2% aqueous mercuric chloride solution, and diethyl ether, then added to the reaction flask. A slight exotherm occurred and the flask was allowed to cool and stir at room temperature for 16 hours. The reaction was filtered through Celite and the filtrate concentrated to yield trans-5-amino-6-(4-bromophenyl)piperidin-2-one as a solid. Yield: 9.39 g, 34.9 mMol, 70%. LCMS m/z 269/271 (M+1). ¹H NMR (400 MHz, CHLOROFORM-d) δ 1.82 (m, 1H), 2.03 (m, 1H), 2.59 (m, 2H), 3.00 (m, 1H), 4.09 (d, J=8.3 Hz, 1H), 7.24 (d, J=8.5 Hz, 2H), 7.54 (d, J=8.3 Hz, 2H).

Step 3: Preparation of trans-N-[2-(4-bromophenyl)-6-oxopiperidin-3-yl]propane-2-sulfonamide. A flask was charged with trans-5-amino-6-(4-bromophenyl)piperidin-2-one (9.39 g, 34.9 mMol) and methylene chloride (100 mL), and 1,8-diazabicyclo[5.4.0]undec-7-ene (DBU) (7.83 mL, 52.3 mMol) was added to the slurry. The reaction was cooled to 0° C. and propane-2-sulfonyl chloride (4.93 mL, 41.9 mMol) was added drop-wise. The reaction was allowed to warm to room temperature and stir for 16 hours, then washed with saturated aqueous sodium chloride solution and dried over magnesium sulfate. The organic layer was filtered and the filtrate concentrated. The crude product was purified by silica gel chromatography (Gradient: 0 to 5% methanol in methylene chloride) to provide trans-N-[2-(4-bromophenyl)-6-oxopiperidin-3-yl]propane-2-sulfonamide as a white solid. Yield: 4.71 g, 12.6 mMol, 36%. LCMS m/z 374.9/376.9 (M+1). ¹H NMR (400 MHz, CHLOROFORM-d) δ 1.27 (d, J=6.6 Hz, 3H), 1.32 (d, J=6.8 Hz, 3H), 1.80 (m, 1H), 2.04 (m, 1H), 2.57 (m, 1H), 2.71 (m, 1H), 3.01 (m, 1H), 3.74 (m, 1H), 4.75 (dd, apparent t, J=3.5, 3.2 Hz, 1H), 6.08 (d, J=9.1 Hz, 1H), 6.70 (br d, J=2.1 Hz, 1H), 7.23 (d, J=8.3 Hz, 2H), 7.53 (d, J=8.5 Hz, 2H).

Step 4: Isolation of N-[(2S,3R)-2-(4-bromophenyl)-6-oxopiperidin-3-yl]propane-2-sulfonamide and N-[(2R,3S)-2-(4-bromophenyl)-6-oxopiperidin-3-yl]propane-2-sulfonamide. The racemic material trans-N-[2-(4-bromophenyl)-6-oxopiperidin-3-yl]propane-2-sulfonamide (4.5 g, 12 mMol) was subjected to chiral HPLC chromatography using a Chiralcel OD-H column, 5 μm, 2.1 cm×25 cm, mobile phase: 80/20 carbon dioxide/methanol at a flow rate of 65 g/minute.

Material with a retention time of 4.57 minutes was collected to yield N-[(2S,3R)-2-(4-bromophenyl)-6-oxopiperidin-3-yl]propane-2-sulfonamide. Yield: 2.14 g, 5.71 mMol, 48%. LCMS m/z 374.9/376.9 (M+1). ¹H NMR (500 MHz, CHLOROFORM-d) δ 1.31 (d, J=6.8 Hz, 3H), 1.35 (d, J=6.8 Hz, 3H), 1.77 (m, 1H), 1.98 (m, 1H), 2.53 (m, 1H), 2.76 (m, 1H), 3.08 (m, 1H), 3.75 (m, 1H), 4.82 (dd, apparent t, J=3.2, 3.2 Hz, 1H), 6.61 (d, J=9.3 Hz, 1H), 6.99 (d, J=2.9 Hz, 1H), 7.23 (d, J=8.5 Hz, 2H), 7.52 (d, J=8.5 Hz, 2H). The absolute configuration of this material was established via X-ray crystallography.

Material with a retention time of 6.61 minutes was collected to yield N-[(2R,3S)-2-(4-bromophenyl)-6-oxopiperidin-3-yl]propane-2-sulfonamide. Yield: 2.17 g, 5.79 mMol, 48%. LCMS m/z 375/377 (M+1). ¹H NMR (500 MHz, CHLOROFORM-d) δ 1.33 (d, J=6.8 Hz, 3H), 1.35 (d, J=6.8 Hz, 3H), 1.76 (m, 1H), 1.96 (m, 1H), 2.52 (m, 1H), 2.78 (m, 1H), 3.11 (m, 1H), 3.75 (m, 1H), 4.85 (br s, 1H), 6.88 (d, J=9.5 Hz, 1H), 7.12 (d, J=2.9 Hz, 1H), 7.23 (d, J=8.5 Hz, 2H), 7.52 (d, J=8.5 Hz, 2H).

Step 5: Preparation of N-[(2S,3R)-2-(2′-ethoxybiphenyl-4-yl)-6-oxopiperidin-3-yl]propane-2-sulfonamide. A mixture of N-[(2S,3R)-2-(4-bromophenyl)-6-oxopiperidin-3-yl]propane-2-sulfonamide (200 mg, 0.533 mMol), (2-ethoxyphenyl)boronic acid (88.5 mg, 0.533 mMol), tetrakis(triphenylphosphine)palladium(0) (31 mg, 0.027 mMol) sodium carbonate (282 mg, 2.66 mMol), degassed methanol (4.0 mL), degassed toluene (0.5 mL), and degassed water (0.5 mL) was evacuated and backfilled with nitrogen three times. The reaction was heated to 80° C. for 16 hours, then concentrated. The residue was partitioned between saturated aqueous sodium chloride solution and methylene chloride, and the organic layer was dried over magnesium sulfate, filtered, and concentrated. The product was purified by silica gel chromatography (Gradient: 0 to 100% ethyl acetate in heptane) to give title compound as a foam. Yield: 95 mg, 0.23 mMol, 43%. LCMS m/z 417.0 (M+1). ¹H NMR (500 MHz, CHLOROFORM-d) δ 1.19 (d, J=6.8 Hz, 3H), 1.29 (d, J=6.6 Hz, 3H), 1.36 (t, J=7.0 Hz, 3H), 1.84 (m, 1H), 2.22 (m, 1H), 2.61 (m, 1H), 2.75 (m, 1H), 2.92 (m, 1H), 3.81 (m, 1H), 4.07 (q, J=7.0 Hz, 2H), 4.74 (m, 1H), 5.91 (d, J=9.0 Hz, 1H), 6.66 (br s, 1H), 6.99 (d, J=8.1 Hz, 1H), 7.03 (dd, apparent t, J=7.4, 7.4 Hz, 1H), 7.31 (d, J=7.8 Hz, 1H), 7.32 (m, 1H), 7.38 (d, J=8.1 Hz, 2H), 7.60 (d, J=8.3 Hz, 2 H).

### Examples 11 and 12

### N-[(2R,3S)-2-biphenyl-4-yl-6-oxopiperidin-3-yl]propane-2-sulfonamide and N-[(2S,3R)-2-biphenyl-4-yl-6-oxopiperidin-3-yl]propane-2-sulfonamide

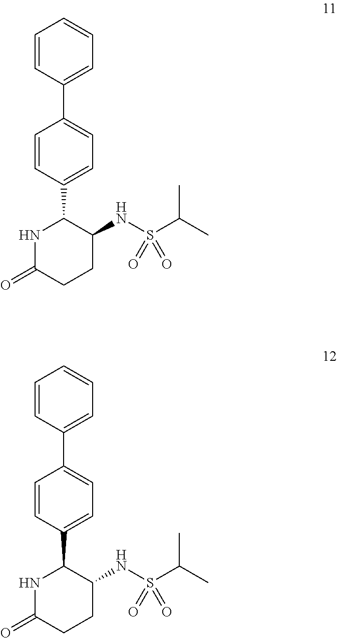

Racemic trans-N-[2-biphenyl-4-yl-6-oxopiperidin-3-yl]propane-2-sulfonamide (prepared using procedures analogous to the preparation of Example 10, except that biphenyl-4-carbaldehyde was employed instead of 4-bromobenzaldehyde) (1.4 g, 3.76 mMol) was subjected to chiral HPLC chromatography using a Chiralcel OD-H column, 5 μm, 1.0 cm×25 cm, mobile phase: 75/25 carbon dioxide/methanol at a flow rate of 10 mL/minute.

Material with a retention time of 7.46 minutes was collected to yield N-[(2R,3S)-2-biphenyl-4-yl-6-oxopiperidin-3-yl]propane-2-sulfonamide. Yield: 0.36 g, 0.97 mMol, 26%. LCMS m/z 373 (M+1). ¹H NMR (400 MHz, CHLOROFORM-d) δ 1.31 (d, J=6.6 Hz, 3H), 1.35 (d, J=6.8 Hz, 3H), 1.80 (m, 1H), 2.09 (m, 1H), 2.57 (m, 1H), 2.80 (m, 1H), 3.08 (m, 1H), 3.84 (m, 1H), 4.92 (br s, 1H), 6.84 (d, J=9.1 Hz, 1H), 7.13 (br s, 1H), 7.41 (m, 5H), 7.59 (m, 4H).

Material with a retention time of 10.8 minutes was collected to yield N-[(2S,3R)-2-biphenyl-4-yl-6-oxopiperidin-3-yl]propane-2-sulfonamide. Yield: 0.43 g, 1.15 mMol, 31%. LCMS m/z 373 (M+1). ¹H NMR (400 MHz, CHLOROFORM-d) δ 1.29 (d, J=6.8 Hz, 3H), 1.34 (d, J=6.6 Hz, 3H), 1.81 (m, 1H), 2.10 (m, 1H), 2.58 (m, 1H), 2.81 (m, 1H), 3.06 (m, 1H), 3.83 (m, 1H), 4.90 (br s, 1H), 6.72 (d, J=9.1 Hz, 1H), 7.05 (br s, 1H), 7.42 (m, 5H), 7.59 (m, 4H). The absolute configurations shown for the title compounds are tentatively assigned.

### Example 13

### N-[(3S,4R)-4-biphenyl-4-yltetrahydro-2H-pyran-3-yl]propane-2-sulfonamide

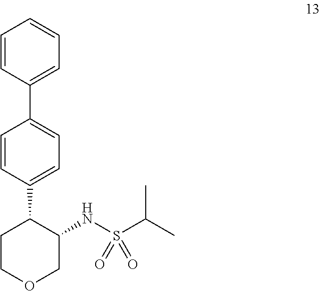

Step 1: Preparation of ethyl 4-phenyl-5,6-dihydro-2H-pyran-3-carboxylate. A flask containing ethyl 4-{[(trifluoromethyl)sulfonyl]oxy}-3,6-dihydro-2H-pyran-3-carboxylate (36.0 g, 118.5 mMol), phenylboronic acid (20.66 g, 169 mMol), potassium carbonate (55.6 g, 402 mMol), and tetrahydrofuran (700 mL) was evacuated and back filled with nitrogen three times. Tetrakis(triphenylphosphine)palladium(0) (13.7 g, 11.9 mMol) was added and the flask was evacuated and back filled with nitrogen three times. The reaction was heated to 65° C. for 4 hours. The cooled reaction was partitioned between ethyl acetate and water. The aqueous layer was re-extracted three times with ethyl acetate and the combined organic layers were dried over sodium sulfate, filtered, and concentrated. The crude product was purified by silica gel chromatography (Gradient: 0 to 20% ethyl acetate in heptane) to give ethyl 4-phenyl-5,6-dihydro-2H-pyran-3-carboxylate as an oil, still contaminated with extraneous aromatic material. Yield: 25 g, <120 mMol, <90%. ¹H NMR (400 MHz, CHLOROFORM-d) δ 0.89 (t, J=7.1 Hz, 3H), 2.49 (m, 2H), 3.89 (t, J=5.5 Hz, 2H), 3.92 (q, J=7.1 Hz, 2H), 4.46 (t, J=2.5 Hz, 2 H), 7.13 (m, 2H), 7.28 (m, 3H).

Step 2: Preparation of ethyl cis-4-phenyltetrahydro-2H-pyran-3-carboxylate. A Parr bottle was charged with ethyl 4-phenyl-5,6-dihydro-2H-pyran-3-carboxylate (10 g, 43 mMol), 10% wt/wt palladium on activated carbon catalyst (6 g), and absolute ethanol (150 mL), and shaken under an atmosphere of hydrogen (50 psi) for 16 hours. The catalyst was removed by filtration and the filtrate concentrated to yield crude ethyl cis-4-phenyltetrahydro-2H-pyran-3-carboxylate, which was generally used in the next step without further purification. Yield: assumed quantitative. ¹H NMR (400 MHz, CHLOROFORM-d) δ 1.01 (t, J=7.1 Hz, 3H), 1.70 (br d, 1H), 2.74 (m, 1H), 2.88 (br s, 1H), 3.07 (ddd, apparent dt, J=12.3, 4.2, 4.2 Hz, 1H), 3.54 (ddd, apparent td, J=11.5, 11.5, 2.5 Hz, 1H), 3.74 (dd, J=11.7, 3.3 Hz, 1 H), 3.93 (m, 2H), 4.17 (m, 1H), 4.28 (br d, J=11.7 Hz, 1H), 7.19 (m, 2H), 7.28 (m, 3H).

Step 3: Preparation of ethyl cis-4-(4-iodophenyl)tetrahydro-2H-pyran-3-carboxylate. A flask was charged with compound ethyl cis-4-phenyltetrahydro-2H-pyran-3-carboxylate (9.0 g, 38.5 mMol), a solution of iodine monochloride (1N in methylene chloride, 154 mL, 154 mMol) was added, and the mixture was heated to 35° C. for 16 hours. Additional iodine monochloride solution was added (50 mL, 50 mMol) and the reaction heated for another 5 hours, at which point additional iodine monochloride solution was added (50 mL, 50 mMol) and the reaction heated for another 16 hours. The reaction was cooled and concentrated, and the residue chromatographed on silica gel (Eluant: 0-80% ethyl acetate in heptane). The later fractions were rechromatographed on silica gel (Eluant: 1-60% ethyl acetate in heptane) to provide ethyl cis-4-(4-iodophenyl)tetrahydro-2H-pyran-3-carboxylate as a solid. Yield: 11.5 g, 31.9 mMol, 83%. LCMS m/z 361.5 (M+1). ¹H NMR (400 MHz, CHLOROFORM-d) δ 1.06 (t, J=7.1 Hz, 3H), 1.68 (br d, J=13.2 Hz, 1H), 2.69 (m, 1H), 2.85 (br s, 1H), 3.01 (ddd, apparent dt, J=12.3, 4.1, 4.1 Hz, 1H), 3.53 (ddd, apparent td, J=11.6, 11.6, 2.4 Hz, 1H), 3.73 (dd, J=11.7, 3.3 Hz, 1H), 3.96 (m, 2H), 4.16 (m, 1H), 4.28 (br d, J=11.7 Hz, 1H), 7.02 (d, J=8.2 Hz, 2H), 7.60 (d, J=8.4 Hz, 2H).

Step 4: Preparation of cis-4-(4-iodophenyl)tetrahydro-2H-pyran-3-carboxylic acid. The ester ethyl cis-4-(4-iodophenyl)tetrahydro-2H-pyran-3-carboxylate (14.5 g, 40.3 mMol), aqueous hydrochloric acid (3N, 544 mL, 1632 mMol,), and 1,4-dioxane (180 mL) were combined and refluxed for 22 hours. The reaction was cooled, and the white solid collected by filtration and dried using a toluene azeotrope to give 11.7 g of cis-4-(4-iodophenyl)tetrahydro-2H-pyran-3-carboxylic acid. The filtrate was concentrated and partitioned between water and methylene chloride. The organics were dried over sodium sulfate, filtered, concentrated, and purified by silica gel chromatography (Eluant: 1-10% methanol in methylene chloride) to afford an additional 3.5 g of cis-4-(4-iodophenyl)tetrahydro-2H-pyran-3-carboxylic acid. Total yield: 15.2 g, assumed quantitative. LCMS m/z 331.4 (M−1). ¹H NMR (400 MHz, METHANOL-d₄) δ 1.69 (br d, J=13.1 Hz, 1H), 2.66 (m, 1H), 2.92 (br s, 1H), 3.08 (ddd, apparent dt, J=12.3, 4.2, 4.2 Hz, 1 H), 3.59 (ddd, apparent td, J=11.6, 11.6, 2.5 Hz, 1H), 3.80 (dd, J=11.7, 3.3 Hz, 1H), 4.12 (m, 1H), 4.26 (br d, J=11.7 Hz, 1H), 7.11 (d, J=8.0 Hz, 2H), 7.62 (d, J=8.6 Hz, 2H).

Step 5: Preparation of cis-4-(4-iodophenyl)tetrahydro-2H-pyran-3-amine. To a solution of cis-4-(4-iodophenyl)tetrahydro-2H-pyran-3-carboxylic acid (9.0 g, 27.1 mMol) in anhydrous toluene (140 mL) was added diisopropylethylamine (7.08 mL, 40.5 mMol) and diphenylphosphoryl azide (DPPA) (8.79 mL, 40.7 mMol). The reaction was stirred at 85° C. for 3.5 hours then allowed to cool to room temperature. Tetrahydrofuran (140 mL) and aqueous sodium hydroxide (2N, 70 mL, 140 mMol) were added and the reaction was stirred for 16 hours. The reaction was diluted with water (150 mL) and extracted four times with ethyl acetate. The combined organics were washed with saturated aqueous sodium chloride solution, dried over sodium sulfate, filtered, and concentrated. The material was triturated with diethyl ether, filtered, and the solids discarded. The filtrate was extracted with aqueous hydrochloric acid (1N). The aqueous layer was basified with aqueous sodium hydroxide (2N), extracted with diethyl ether and then with methylene chloride. The combined organics were dried over sodium sulfate, filtered, and concentrated to afford cis-4-(4-iodophenyl)tetrahydro-2H-pyran-3-amine as a solid. Yield: 9.5 g, assumed quantitative. LCMS m/z 304.0 (M+1). ¹H NMR (400 MHz, METHANOL-d₄) δ 1.49 (br d, J=13.6 Hz, 1H), 2.25 (m, 1 H), 2.90 (br s, 1H), 3.02 (ddd, apparent dt, J=13.1, 3.4, 3.4 Hz, 1H), 3.49 (ddd, apparent td, J=11.8, 11.8, 2.2 Hz, 1H), 3.68 (dd, J=11.5, 2.0 Hz, 1H), 3.86 (d, J=11.5 Hz, 1H), 4.02 (dd, J=11.3, 4.5 Hz, 1H), 7.02 (d, J=8.2 Hz, 2 H), 7.65 (d, J=8.4 Hz, 2H).

Step 6: Preparation of cis-N-[4-(4-iodophenyl)tetrahydro-2H-pyran-3-yl]propane-2-sulfonamide. A flask was charged with a mixture of cis-4-(4-iodophenyl)tetrahydro-2H-pyran-3-amine (5.2 g, 17.2 mMol), 1,8-diazabicyclo[5.4.0]undec-7-ene (DBU) (5.13 mL, 34.3 mMol), and methylene chloride (50 mL), and placed in a 0° C. cooling bath. Propane-2-sulfonyl chloride (3.83 mL, 34.3 mMol) was added drop-wise and the reaction was allowed to warm to room temperature and stir for 16 hours. The reaction was combined with a similar crude reaction mixture from subjection of 5.0 g of cis-4-(4-iodophenyl)tetrahydro-2H-pyran-3-amine to the same procedure. The combined reactions were concentrated, then partitioned between methylene chloride and water. The aqueous phase was re-extracted three times with methylene chloride. The combined organics were washed with saturated aqueous sodium chloride solution, dried over sodium sulfate, filtered, and concentrated. The crude product was triturated with methylene chloride and the solid collected by filtration to afford pure cis-N-[4-(4-iodophenyl)tetrahydro-2H-pyran-3-yl]propane-2-sulfonamide. Yield: 8.9 g, 21.7 mMol, 64%. LCMS m/z 410.0 (M+1). ¹H NMR (400 MHz, CHLOROFORM-d) δ 0.90 (d, J=6.8 Hz, 3H), 1.09 (d, J=6.8 Hz, 3H), 1.72 (br d, J=13.3 Hz, 1H), 2.09 (m, 1H), 2.29 (m, 1H), 3.03 (ddd, apparent dt, J=13.2, 3.5, 3.5 Hz, 1H), 3.55 (ddd, apparent td, J=11.9, 11.9, 2.3 Hz, 1H), 3.71 (m, 2H), 4.14 (m, 2H), 4.52 (d, J=9.8 Hz, 1H), 7.01 (d, J=8.2 Hz, 2H), 7.69 (d, J=8.4 Hz, 2H).

Step 7: Isolation of N-[(3S,4R)-4-(4-iodophenyl)tetrahydro-2H-pyran-3-yl]propane-2-sulfonamide and N-[(3R,4S)-4-(4-iodophenyl)tetrahydro-2H-pyran-3-yl]propane-2-sulfonamide. Racemic compound cis-N-[4-(4-iodophenyl)tetrahydro-2H-pyran-3-yl]propane-2-sulfonamide (8.7 g, 21.3 mMol) was subjected to chiral HPLC chromatography using a Chiralcel OJ-H column, 5 μm, 250 mm×21.0 mm, mobile phase: 85/15 carbon dioxide/methanol at a flow rate of 65 g/minute.

Material with a retention time of 2.65 minutes was collected to yield N-[(3S,4R)-4-(4-iodophenyl)tetrahydro-2H-pyran-3-yl]propane-2-sulfonamide as a solid. Yield: 3.2 g, 7.82 mMol, 37%. LCMS m/z 410.0 (M+1). ¹H NMR (400 MHz, CHLOROFORM-d) δ 0.89 (d, J=6.6 Hz, 3H), 1.09 (d, J=6.6 Hz, 3H), 1.72 (br d, J=14.1 Hz, 1H), 2.09 (m, 1H), 2.28 (m, 1H), 3.05 (m, 1H), 3.54 (dd, apparent t, J=11.9, 11.9 Hz, 1H), 3.70 (m, 2H), 4.13 (m, 2H), 4.58 (d, J=9.6 Hz, 1H), 7.01 (d, J=8.0 Hz, 2H), 7.69 (d, J=8.2 Hz, 2H). The absolute configuration of N-[(3S,4R)-4-(4-iodophenyl)tetrahydro-2H-pyran-3-yl]propane-2-sulfonamide was established via X-ray crystallography.

Material with a retention time of 3.56 minutes was collected to yield N-[(3R,4S)-4-(4-iodophenyl)tetrahydro-2H-pyran-3-yl]propane-2-sulfonamide as a solid. Yield: 3.3 g, 8.06 mMol, 38%. LCMS m/z 410.0 (M+1). ¹H NMR (400 MHz, CHLOROFORM-d) δ 0.89 (d, J=6.8 Hz, 3H), 1.09 (d, J=7.0 Hz, 3H), 1.72 (br d, J=13.7 Hz, 1H), 2.09 (m, 1H), 2.29 (m, 1H), 3.03 (m, 1H), 3.55 (ddd, apparent td, J=11.9, 11.9, 2.2 Hz, 1H), 3.69 (m, 2H), 4.14 (m, 2H), 4.55 (d, J=9.6 Hz, 1H), 7.01 (d, J=8.4 Hz, 2H), 7.69 (d, J=8.4 Hz, 2H).

Step 8: Preparation of N-[(3S,4R)-4-biphenyl-4-yltetrahydro-2H-pyran-3-yl]propane-2-sulfonamide. A vial charged with N-[(3S,4R)-4-(4-iodophenyl)tetrahydro-2H-pyran-3-yl]propane-2-sulfonamide (100 mg, 0.24 mMol), phenylboronic acid (47 mg, 0.383 mMol), 2-dicyclohexylphosphino-2′,4′,6′-triisopropyl-1,1′-biphenyl (X-phos) (11.4 mg, 0.024 mMol), palladium(II) acetate (3.6 mg, 0.016 mMol), potassium fluoride (77 mg, 1.32 mMol), methanol (0.75 mL), and toluene (0.75 mL) was evacuated under vacuum then filled with nitrogen three times. The reaction was heated to 130° C. in a microwave reactor for 30 minutes. The reaction was concentrated and partitioned between ethyl acetate and saturated aqueous sodium chloride solution, the aqueous phase extracted twice with ethyl acetate, and the combined organics were dried and concentrated in vacuo. The crude product was purified by silica gel chromatography (Gradient: 0 to 80% ethyl acetate in heptane) to give pure N-[(3S,4R)-4-biphenyl-4-yltetrahydro-2H-pyran-3-yl]propane-2-sulfonamide. Yield: 75 mg, 0.209 mMol, 87%. LCMS m/z 360.1 (M+1). ¹H NMR (500 MHz, CHLOROFORM-d) δ 0.83 (d, J=6.7 Hz, 3H), 1.04 (d, J=6.8 Hz, 3H), 1.81 (br d, J=13.7 Hz, 1H), 2.19 (m, 2H), 3.14 (ddd, apparent dt, J=13.1, 3.5, 3.5 Hz, 1H), 3.59 (ddd, apparent td, J=11.9, 11.9, 2.2 Hz, 1H), 3.77 (m, 2H), 4.18 (m, 2H), 4.53 (d, J=9.4 Hz, 1H), 7.33 (d, J=8.1 Hz, 2H), 7.37 (m, 1H), 7.46 (m, 2H), 7.58 (m, 4H).

### Example 14

### Preparation of N-[(2R,3R)-2-biphenyl-4-yl-6-oxopiperidin-3-yl]propane-2-sulfonamide (14)

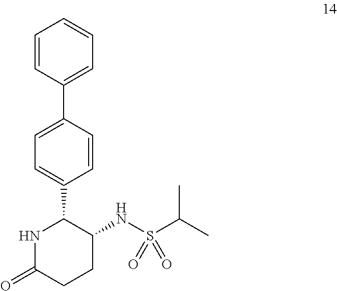

Step 1: Preparation of 6-(4-bromophenyl)piperidine-2,5-dione. A flask was charged with trans-6-(4-bromophenyl)-5-nitropiperidin-2-one (15.2 g, 50.8 mMol) and methylene chloride (200 mL). Potassium tert-butoxide (5.7 g, 50.8 mMol) was added followed by addition of methanol (200 mL), and the mixture was stirred at room temperature for 15 minutes. The reaction was cooled to −78° C. and a stream of ozone was passed through the mixture for 90 minutes. Dimethyl sulfide (20 mL, 272 mMol) was added and the reaction was allowed to slowly warm to room temperature and stir for 16 hours. The reaction was concentrated in vacuo and partitioned between water and methylene chloride. The organics were dried over magnesium sulfate, filtered, and concentrated. The crude product was combined with the crude product from a similar scale reaction and the material was purified by silica gel chromatography (Eluant: 5% methanol in methylene chloride) to provide 6-(4-bromophenyl)piperidine-2,5-dione as a white solid. Yield: 15.56 g, 58 mMol, 57%. ¹H NMR (500 MHz, CHLOROFORM-d) δ 2.76 (m, 4H), 4.96 (d, J=2.4 Hz, 1H), 6.26 (br s, 1H), 7.27 (d, J=8.8 Hz, 2H), 7.55 (d, J=8.5 Hz, 2H).

Step 2: Preparation of 6-(4-bromophenyl)piperidine-2,5-dione 5-oxime. A flask was charged with compound 6-(4-bromophenyl)piperidine-2,5-dione (14.42 g, 53.78 mMol) and ethanol (300 mL) to form a slurry. In a separate flask hydroxylamine hydrochloride (11.3 g, 156 mMol) and sodium acetate (22.7 g, 269 mMol) were dissolved in water (100 mL). The aqueous solution was added to the slurry and the mixture was stirred for 16 hours. The reaction was concentrated at reduced pressure to approximately 100 mL volume and this was poured into ice water (350 mL). The resulting precipitate was filtered and washed with diethyl ether to give the product 6-(4-bromophenyl)piperidine-2,5-dione 5-oxime. Yield: 13.55 g, 47.9 mMol, 89%. ¹H NMR (500 MHz, DMSO-d₆, major and minor oxime isomers in 85:15 ratio) major isomer: δ 2.15 (m, 1H), 2.25 (m, 1H), 2.49 (m, 1H assumed, obscured by solvent), 2.73 (m, 1H), 5.04 (d, J=4.1 Hz, 1H), 7.25 (d, J=8.3 Hz, 2H), 7.58 (d, J=8.5 Hz, 2H), 8.35 (d, J=4.1 Hz, 1H), 11.06 (s, 1H); minor isomer, selected peaks: 5.69 (d, J=2.9 Hz, 1H), 7.34 (d, J=8.3 Hz, 2 H), 7.56 (d, J=8.5 Hz, 2H), 8.06 (d, J=2.7 Hz, 1H), 11.11 (s, 1H).

Step 3: Preparation of cis-5-amino-6-(4-bromophenyl)piperidin-2-one. A Parr bottle was charged with 6-(4-bromophenyl)piperidine-2,5-dione 5-oxime (7.1 g, 25.0 mMol), absolute ethanol (100 mL), and Raney Nickel (estimated 3-5 g, washed several times with water and then once with ethanol) and shaken under an atmosphere of hydrogen (45 psi) for 16 hours. The catalyst was removed by filtration through Celite and the filtrate concentrated to give the crude product cis-5-amino-6-(4-bromophenyl)piperidin-2-one, contaminated with a roughly equimolar amount of its des-bromo analogue cis-5-amino-6-phenylpiperidin-2-one. Yield: 5.42 g, approximately 11.8 mMol of the title product, approximately 47% yield. LCMS m/z 271/269 (M+1). ¹H NMR (400 MHz, METHANOL-d₄) δ 1.83-2.05 (m, 2H from each compound), 2.43-2.63 (m, 2H from each compound), 3.42-3.51 (m, 1H from each compound), 4.72 (d, J=4.5 Hz, 1H from cis-5-amino-6-(4-bromophenyl)piperidin-2-one), 4.77 (d, J=4.7 Hz, 1H from cis-5-amino-6-phenylpiperidin-2-one), 7.27 (d, J=8.4 Hz, 2H from cis-5-amino-6-(4-bromophenyl)piperidin-2-one), 7.33-7.47 (m, 5H from cis-5-amino-6-phenylpiperidin-2-one), 7.59 (d, J=8.4 Hz, 2H from cis-5-amino-6-(4-bromophenyl)piperidin-2-one).

Step 4: Preparation of cis-N-[2-(4-bromophenyl)-6-oxopiperidin-3-yl]propane-2-sulfonamide. A flask was charged with a mixture of cis-5-amino-6-(4-bromophenyl)piperidin-2-one from the previous step (1.5 g, approximately 3.3 mMol of substrate), 1,8-diazabicyclo[5.4.0]undec-7-ene (DBU) (1.25 mL, 8.36 mMol), and methylene chloride (15 mL), and placed in a 0° C. cooling bath. Propane-2-sulfonyl chloride (0.984 mL, 8.36 mMol) was added drop-wise and the reaction was allowed to warm slowly to room temperature and stir for 16 hours. The reaction was diluted with saturated aqueous sodium chloride solution and the phases separated. The organic phase was dried over magnesium sulfate, filtered, and concentrated. The product was purified by silica gel chromatography (Eluant: 5% methanol in methylene chloride) to provide cis-N-[2-(4-bromophenyl)-6-oxopiperidin-3-yl]propane-2-sulfonamide as a solid containing a roughly equimolar quantity of its des-bromo analogue cis-6-oxo-2-phenylpiperidin-3-yl]propane-2-sulfonamide. Yield: 674 mg, approximately 1.00 mMol of the title product, approximately 30%. LCMS m/z 375 (M−1). ¹H NMR (500 MHz, CHLOROFORM-d) δ 1.10, 1.19, 1.22 and 1.25 (4 doublets, J=6.6, 6.8, 6.8, 6.8 Hz, total of 6H from each compound), 1.92-2.15 (m, 4H), 2.55-2.73 (m, 4 H), 2.90 (m, 1H), 2.97 (m, 1H), 3.95-4.01 (m, 2H), 4.16 and 4.27 (2 doublets, J=9.5, 10.0 Hz, 1H from each compound), 4.83 and 4.86 (2 multiplets, 1H from each compound), 6.02 and 6.07 (2 broad singlets, 1H from each compound), 7.24 (d, J=8.5 Hz, 2H from cis-N-[2-(4-bromophenyl)-6-oxopiperidin-3-yl]propane-2-sulfonamide), 7.34-7.46 (m, 5H from cis-6-oxo-2-phenylpiperidin-3-yl]propane-2-sulfonamide), 7.57 (d, J=8.5 Hz, 2H from cis-N-[2-(4-bromophenyl)-6-oxopiperidin-3-yl]propane-2-sulfonamide).

Step 5: Isolation of N-[(2S,3S)-2-(4-bromophenyl)-6-oxopiperidin-3-yl]propane-2-sulfonamide and N-[(2R,3R)-2-(4-bromophenyl)-6-oxopiperidin-3-yl]propane-2-sulfonamide. Racemic compound cis-N-[2-(4-bromophenyl)-6-oxopiperidin-3-yl]propane-2-sulfonamide (936 mg of a mixture similar to that isolated in the previous step, approximately 1.39 mMol desired substrate) was subjected to chiral HPLC chromatography using a Chiralcel OJ-H column, 5 uM, 2.1 cm×25 cm, mobile phase: 85/15 mixture of carbon dioxide/methanol at a flow rate of 65 g/minute. Material with a retention time of 5.94 minutes was collected to give N-[(2S,3S)-2-(4-bromophenyl)-6-oxopiperidin-3-yl]propane-2-sulfonamide as a solid. Yield: 240 mg, 0.639 mMol, approximately 46%. ¹H NMR (400 MHz, CHLOROFORM-d) δ 1.19 (d, J=6.8 Hz, 3H), 1.26 (d, J=6.8 Hz, 3H), 1.91-2.06 (m, 2H), 2.56-2.72 (m, 2H), 2.97 (m, 1H), 3.97 (m, 1H), 4.13 (d, J=10.0 Hz, 1H), 4.83 (dd, J=4.5, 2.7 Hz, 1 H), 6.06 (br s, 1H), 7.24 (d, J=8.4 Hz, 2H), 7.58 (d, J=8.4 Hz, 2H).

Material with a retention time of 7.11 minutes was collected to yield N-[2R,3R)-2-(4-bromophenyl)-6-oxopiperidin-3-yl]propane-2-sulfonamide as a solid. Yield: 284 mg, 0.775 mMol, approximately 50%. ¹H NMR (400 MHz, CHLOROFORM-d) δ 1.20 (d, J=6.6 Hz, 3H), 1.27 (d, J=6.8 Hz, 3H), 1.91-2.03 (m, 2H), 2.57-2.72 (m, 2H), 2.98 (m, 1H), 3.98 (m, 2H), 4.84 (m, 1H), 5.96 (br s, 1H), 7.24 (d, J=8.4 Hz, 2H), 7.58 (d, J=8.4 Hz, 2H). The absolute configuration of this material was established via X-ray crystallography.

Step 6: Preparation of N-[(2R,3R)-2-biphenyl-4-yl-6-oxopiperidin-3-yl]propane-2-sulfonamide. A vial charged with N-[(2R,3R)-2-(4-bromophenyl)-6-oxopiperidin-3-yl]propane-2-sulfonamide (211 mg, 0.562 mMol), phenylboronic acid (71.9 mg, 0.59 mMol), 2-dicyclohexylphosphino-2′,4′,6′-triisopropyl-1,1′-biphenyl (X-phos) (25.7 mg, 0.054 mMol), palladium(II) acetate (8.3 mg, 0.037 mMol), potassium fluoride (163 mg, 2.81 mMol), methanol (1.0 mL), and toluene (1.0 mL)) was evacuated under vacuum then filled with nitrogen three times. The reaction was heated to 120° C. in a microwave reactor for 20 minutes. The reaction was filtered and the filtrate was concentrated and partitioned between methylene chloride and saturated aqueous sodium chloride solution. The organic phase was dried over magnesium sulfate, filtered, and concentrated. The crude product was purified by silica gel chromatography (Gradient: 0 to 100% ethyl acetate in heptane) to give pure N-[(2R,3R)-2-biphenyl-4-yl-6-oxopiperidin-3-yl]propane-2-sulfonamide as a white solid. Yield: 65 mg, 0.175 mMol, 31%. LCMS m/z 371.1 (M−1). ¹H NMR (500 MHz, CHLOROFORM-d) δ 1.14 (d, J=6.8 Hz, 3 H), 1.24 (d, J=6.8 Hz, 3H), 1.99 (m, 1H), 2.12 (m, 1H), 2.62 (m, 1H), 2.72 (m, 1H), 2.94 (m, 1H), 4.01 (m, 1H), 4.16 (d, J=9.8 Hz, 1H), 4.90 (m, 1H), 6.08 (br s, 1H), 7.39 (br t, J=7.3 Hz, 1H), 7.42 (d, J=8.3 Hz, 2H), 7.47 (broad dd, apparent br t, J=7.6, 7.6 Hz, 2H), 7.59 (br d, J=8.3 Hz, 2H), 7.67 (d, J=8.3 Hz, 2H).

Additional Examples are provided in Table 1

| Ex. | Structure | IUPAC Name | Physical Data |
|---|---|---|---|
| 15 | 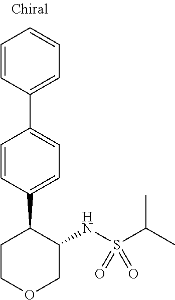 | N-[(3S,4S)-4- biphenyl-4- yltetrahydro- 2H-pyran-3- yl]propane-2- sulfonamide | LCMS m/z 358.1 (M − 1). ¹H NMR (400 MHz, CHLOROFORM-d) δ 0.64 (d, J = 6.8 Hz, 3 H), 1.11 (d, J = 6.8 Hz, 3 H), 1.92 (m, 1 H), 2.06 (m, 1 H), 2.58 (m, 2 H), 3.23 (dd, apparent t, J = 10.7, 10.7 Hz, 1 H), 3.49 (ddd, apparent td, J = 11.7, 11.7, 2.1 Hz, 1 H), 3.53 (m, 1 H), 4.06 (m, 2 H), 4.41 (dd, J = 11.1, 4.7 Hz, 1 H), 7.35 (d, J = 8.3 Hz, 2 H), 7.38 (m, 1 H), 7.46 (dd, apparent t, J = 7.3, 7.3 Hz, 2 H), 7.57 (d, J = 7.3 Hz, 2 H), 7.59 (d, J = 8.3 Hz, 2 H). Retention time (HPLC)¹ 3.10 minutes |
| 16 | 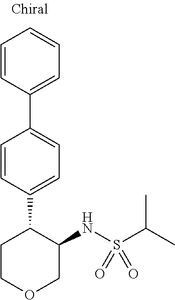 | N-[(3R,4R)-4- biphenyl-4- yltetrahydro- 2H-pyran-3- yl]propane-2- sulfonamide | LCMS m/z 358.1 (M − 1). ¹H NMR (400 MHz, CHLOROFORM-d) δ 0.64 (d, J = 6.8 Hz, 3 H), 1.11 (d, J = 6.8 Hz, 3 H), 1.91 (m, 1 H), 2.06 (m, 1 H), 2.58 (m, 2 H), 3.23 (dd, apparent t, J = 10.7, 10.7 Hz, 1 H), 3.49 (ddd, apparent td, J = 11.8, 11.8, 2.3 Hz, 1 H), 3.53 (m, 1 H), 4.06 (dd, J = 11.6, 3.7 Hz, 1 H), 4.14 (d, J = 7.9 Hz, 1 H), 4.41 (dd, J = 11.1, 4.7 Hz, 1 H), 7.35 (d, J = 8.1 Hz, 2 H), 7.38 (m, 1 H), 7.46 (dd, apparent t, J = 7.6, 7.6 Hz, 2 H), 7.57 (d, J = 7.1 Hz, 2 H), 7.59 (d, J = 8.3 Hz, 2 H). Retention time (HPLC)¹ 6.20 minutes |
| 17 | 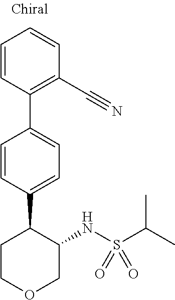 | N-[(3S,4S)-4- (2′-cyano biphenyl-4- yl)tetrahydro- 2H-pyran-3- yl]propane-2- sulfonamide | LCMS m/z 383.1 (M − 1). ¹H NMR (400 MHz, CHLOROFORM-d) δ 0.72 (d, J = 6.6 Hz, 3 H), 1.12 (d, J = 6.8 Hz, 3 H), 1.93 (m, 1 H), 2.06 (m, 1 H), 2.61 (m, 2 H), 3.23 (dd, apparent t, J = 10.7, 10.7 Hz, 1 H), 3.49 (ddd, apparent td, J = 11.8, 11.8, 2.3 Hz, 1 H), 3.58 (m, 1 H), 4.07 (m, 2 H), 4.41 (dd, J = 11.2, 4.8 Hz, 1 H), 7.42 (d, J = 8.3 Hz, 2 H), 7.48 (d, J = 7.3 Hz, 2 H), 7.57 (d, J = 8.3 Hz, 2 H), 7.67 (m, 1 H), 7.78 (d, J = 7.7 Hz, 1 H). Retention time (HPLC)¹ 2.99 minutes |
| 18 | 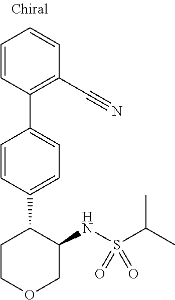 | N-[(3R,4R)-4- (2′-cyano biphenyl-4- yl)tetrahydro- 2H-pyran-3- yl]propane-2- sulfonamide | LCMS m/z 383.1 (M − 1). ¹H NMR (400 MHz, CHLOROFORM-d) δ 0.72 (d, J = 6.6 Hz, 3 H), 1.13 (d, J = 6.8 Hz, 3 H), 1.93 (m, 1 H), 2.06 (m, 1 H), 2.60 (m, 2 H), 3.23 (dd, apparent t, J = 10.7, 10.7 Hz, 1 H), 3.49 (ddd, apparent td, J = 11.8, 11.8, 2.1 Hz, 1 H), 3.58 (m, 1 H), 3.99 (d, J = 8.5 Hz, 1 H), 4.07 (dd, J = 11.5, 3.6 Hz, 1 H), 4.41 (dd, J = 11.2, 4.8 Hz, 1 H), 7.42 (d, J = 8.1 Hz, 2 H), 7.48 (m, 2 H), 7.57 (d, J = 8.3 Hz, 2 H), 7.67 (m, 1 H), 7.79 (d, J = 7.9 Hz, 1 H). Retention time (HPLC)¹ 6.95 minutes |
| 19 | 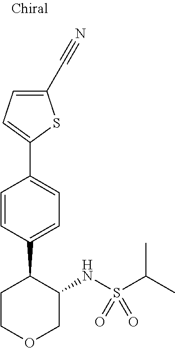 | N-{(3S,4S)-4- [4-(5-cyano-2- thienyl)phenyl] tetrahydro-2H- pyran-3- yl}propane-2- sulfonamide | LCMS m/z 389.0 (M − 1). ¹H NMR (400 MHz, CHLOROFORM-d) δ 0.76 (d, J = 6.8 Hz, 3 H), 1.13 (d, J = 6.8 Hz, 3 H), 1.89 (m, 1 H), 2.01 (m, 1 H), 2.62 (m, 2 H), 3.22 (dd, apparent t, J = 10.7, 10.7 Hz, 1 H), 3.48 (ddd, apparent td, J = 11.8, 11.8, 2.2 Hz, 1 H), 3.58 (m, 1 H), 3.96 (d, J = 7.9 Hz, 1 H), 4.05 (dd, J = 11.7, 3.2 Hz, 1 H), 4.37 (dd, J = 11.2, 4.6 Hz, 1 H), 7.28 (d, J = 3.9 Hz, 1 H), 7.35 (d, J = 8.3 Hz, 2 H), 7.61 (d, J = 3.9 Hz, 1 H), 7.61 (d, J = 8.3 Hz, 2 H). Retention time (HPLC)² 3.22 minutes |
| 20 | 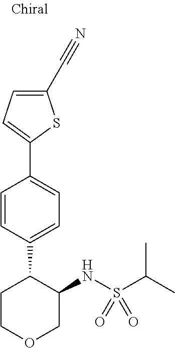 | N-{(3R,4R)-4- [4-(5-cyano-2- thienyl)phenyl] tetrahydro-2H- pyran-3- yl}propane-2- sulfonamide | LCMS m/z 389.0 (M − 1). ¹H NMR (400 MHz, CHLOROFORM-d) δ 0.78 (d, J = 6.6 Hz, 3 H), 1.14 (d, J = 6.8 Hz, 3 H), 1.89 (m, 1 H), 2.01 (m, 1 H), 2.60 (ddd, apparent td, J = 11.4, 11.4, 4.2 Hz, 1 H), 2.64 (m, 1 H), 3.22 (dd, apparent t, J = 10.6, 10.6 Hz, 1 H), 3.48 (ddd, apparent td, J = 11.8, 11.8, 2.3 Hz, 1 H), 3.59 (ddd, apparent td, J = 10.6, 10.6, 4.8 Hz, 1 H), 3.84 (br s, 1 H), 4.06 (dd, J = 11.5, 3.2 Hz, 1 H), 4.38 (dd, J = 11.1, 4.7 Hz, 1 H), 7.28 (d, J = 3.9 Hz, 1 H), 7.35 (d, J = 8.3 Hz, 2 H), 7.61 (d, J = 3.9 Hz, 1 H), 7.61 (d, J = 8.3 Hz, 2 H). Retention time (HPLC)² 9.90 minutes |
| 21 | 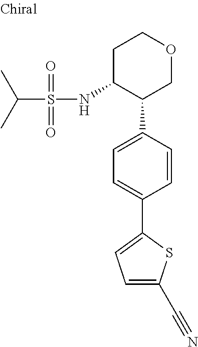 | N-{(3R,4R)-3- [4-(5-cyano-2- thienyl)phenyl] tetrahydro-2H- pyran-4- yl}propane-2- sulfonamide | LCMS m/z 389 (M − 1). ¹H NMR (400 MHz, CHLOROFORM-d) δ 1.27 (d, J = 6.8 Hz, 3 H), 1.30 (d, J = 6.8 Hz, 3 H), 1.80 (m, 2 H), 3.06 (m, 1 H), 3.15 (m, 1 H), 3.64 (m, 2 H), 3.86 (m, 2 H), 4.11 (m, 1 H), 4.18 (dd, J = 12.0, 2.5 Hz, 1 H), 7.28 (d, J = 3.9 Hz, 1 H), 7.59 (d, J = 7.9, 2 H), 7.60 (m, 1 H), 7.66 (d, J = 8.5 Hz, 2 H) |
| 22 | 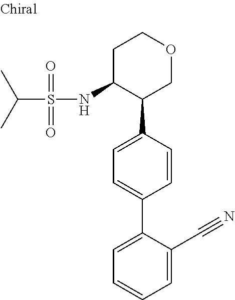 | N-[(3S,4S)-3- (2′-cyano biphenyl-4- yl)tetrahydro- 2H-pyran-4- yl]propane-2- sulfonamide | LCMS m/z 385 (M + 1). ¹H NMR (400 MHz, CHLOROFORM-d) δ 1.19 (d, J = 6.6 Hz, 3 H), 1.28 (d, J = 6.4 Hz, 3 H), 1.92 (m, 2 H), 2.98 (m, 1 H), 3.20 (m, 1 H), 3.70 (m, 1 H), 3.91 (m, 2 H), 4.10 (m, 2 H), 4.19 (dd, J = 11.8, 4.1 Hz, 1 H), 7.47 (m, 2 H), 7.53 (d, J = 8.1 Hz, 2 H), 7.61 (d, J = 7.7 Hz, 2 H), 7.63 (m, 1 H), 7.77 (d, J = 7.7 Hz, 1 H) |
| 23 | 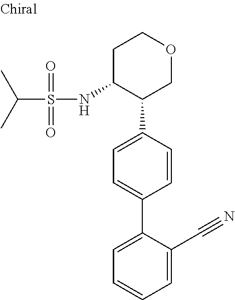 | N-[(3R,4R)-3- (2′-cyano biphenyl-4- yl)tetrahydro- 2H-pyran-4- yl]propane-2- sulfonamide | LCMS m/z 385 (M + 1). ¹H NMR (400 MHz, CHLOROFORM-d) δ 1.23 (d, J = 7.1 Hz, 3 H), 1.30 (d, J = 6.6 Hz, 3 H), 1.84 (m, 1 H), 1.95 (m, 1 H), 3.01 (m, 1 H), 3.21 (m, 1 H), 3.71 (m, 1 H), 3.90 (m, 2 H), 3.94 (dd, J = 12.0, 3.3 Hz, 1 H), 4.08 (m, 1 H), 4.20 (dd, J = 12.0, 4.1 Hz, 1 H), 7.49 (m, 2 H), 7.57 (d, J = 8.3 Hz, 2 H), 7.63 (d, J = 8.3 Hz, 2 H), 7.65 (m, 1 H), 7.78 (dd, J = 7.9, 0.9 Hz, 1 H) |
| 24 | 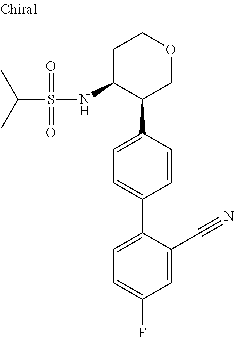 | N-[(3S,4S)-3- (2′-cyano-4′- fluorobiphenyl- 4-yl)tetra hydro-2H- pyran-4-yl] propane-2- sulfonamide | LCMS m/z 401 (M − 1). ¹H NMR (400 MHz, CHLOROFORM-d) δ 1.22 (d, J = 6.6 Hz, 3 H), 1.29 (d, J = 6.6 Hz, 3 H), 1.89 (m, 2 H), 3.01 (m, 1 H), 3.20 (m, 1 H), 3.70 (m, 1 H), 3.92 (m, 3 H), 4.07 (m, 1 H), 4.19 (dd, J = 11.8, 3.9 Hz, 1 H), 7.38 (ddd, J = 7.9, 7.9, 2.8 Hz, 1 H), 7.48 (m, 2 H), 7.52 (d, J = 8.3 Hz, 2 H), 7.65 (d, J = 8.3 Hz, 2 H) |
| 25 | 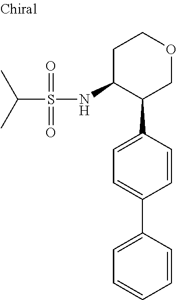 | N-[(3S,4S)-3- biphenyl-4- yltetrahydro- 2H-pyran-4- yl]propane-2- sulfonamide | LCMS m/z 360.0 (M + 1). ¹H NMR (400 MHz, CHLOROFORM- d) δ 1.23 (d, J = 6.8 Hz, 3 H), 1.31 (d, J = 6.8 Hz, 3 H), 1.83 (m, 1 H), 1.95 (m, 1 H), 3.04 (m, 1 H), 3.17 (m, 1 H), 3.70 (m, 1 H), 3.79 (d, J = 9.8 Hz, 1 H), 3.87 (m, 1 H), 3.94 (dd, J = 12.0, 3.3 Hz, 1 H), 4.10 (m, 1 H), 4.21 (dd, J = 11.9, 3.6 Hz, 1 H), 7.38 (m, 1 H), 7.46 (m, 2 H), 7.59 (m, 6 H) |
| 26 | 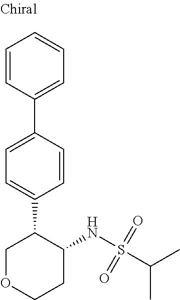 | N-[(3R,4R)-3- biphenyl-4- yltetrahydro- 2H-pyran-4- yl]propane-2- sulfonamide | LCMS m/z 360.0 (M + 1). ¹H NMR (400 MHz, CHLOROFORM- d) δ 1.23 (d, J = 6.8 Hz, 3 H), 1.31 (d, J = 6.8 Hz, 3 H), 1.83 (m, 1 H), 1.95 (m, 1 H), 3.04 (m, 1 H), 3.17 (m, 1 H), 3.70 (m, 1 H), 3.79 (d, J = 9.8 Hz, 1 H), 3.87 (m, 1 H), 3.94 (dd, J = 12.0, 3.3 Hz, 1 H), 4.10 (m, 1 H), 4.21 (dd, J = 11.8, 3.7 Hz, 1 H), 7.38 (m, 1 H), 7.46 (m, 2 H), 7.59 (m, 6H) |
| 27 | 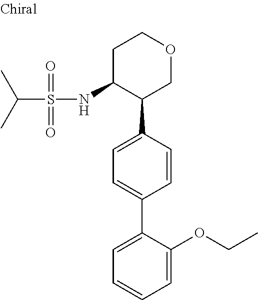 | N-[(3S,4S)-3- (2′-ethoxy biphenyl-4- yl)tetrahydro- 2H-pyran-4- yl]propane-2- sulfonamide | LCMS m/z 404 (M + 1). ¹H NMR (400 MHz, CHLOROFORM-d) δ 1.18 (d, J = 6.8 Hz, 3 H), 1.30 (d, J = 6.8 Hz, 3 H), 1.38 (t, J = 6.9 Hz, 3 H), 1.86 (m, 1 H), 2.00 (m, 1 H), 3.00 (m, 1 H), 3.16 (m, 1 H), 3.71 (ddd, J = 11.9, 9.1, 3.1 Hz, 1 H), 3.89 (m, 2 H), 3.95 (dd, J = 11.9, 3.4 Hz, 1 H), 4.07 (m, 1 H), 4.08 (d, J = 7.0 Hz, 2 H), 4.20 (dd, J = 11.8, 4.4 Hz, 1 H), 6.99 (d, J = 8.3 Hz, 1 H), 7.03 (ddd, apparent td, J = 7.5, 7.5, 0.8 Hz, 1 H), 7.32 (m, 2 H), 7.52 (d, J = 8.3 Hz, 2 H), 7.58 (d, J = 8.3 Hz, 2 H) |
| 28 | 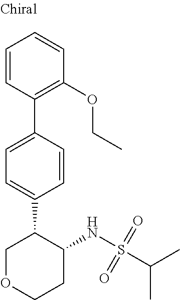 | N-[(3R,4R)-3- (2′-ethoxy biphenyl-4- yl)tetrahydro- 2H-pyran-4- yl]propane-2- sulfonamide | LCMS m/z 404 (M + 1). ¹H NMR (400 MHz, CHLOROFORM-d) δ 1.18 (d, J = 6.6 Hz, 3 H), 1.30 (d, J = 7.1 Hz, 3 H), 1.38 (t, J = 7.1 Hz, 3 H), 1.86 (m, 1 H), 2.00 (m, 1 H), 3.00 (m, 1 H), 3.16 (m, 1 H), 3.72 (ddd, J = 12.0, 9.1, 2.9 Hz, 1 H), 3.89 (m, 2 H), 3.95 (dd, J = 12.0, 3.3 Hz, 1 H), 4.07 (m, 1 H), 4.08 (q, J = 7.1 Hz, 2 H), 4.20 (dd, J = 11.8, 4.4 Hz, 1 H), 6.99 (d, J = 7.9 Hz, 1 H), 7.03 (dd, apparent t, J = 7.5, 7.5 Hz, 1 H), 7.32 (m, 2 H), 7.52 (d, J = 7.9 Hz, 2 H), 7.58 (d, J = 8.3 Hz, 2 H) |
| 29 | 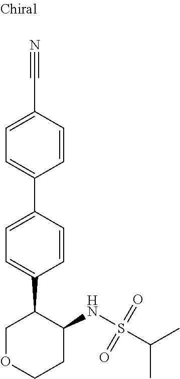 | N-[(3S,4S)-3- (4′-cyano biphenyl-4- yl)tetrahydro- 2H-pyran-4- yl]propane-2- sulfonamide | LCMS m/z 383.1 (M − 1). ¹H NMR (400 MHz, CHLOROFORM-d) δ 1.28 (d, J = 6.6 Hz, 3 H), 1.32 (d, J = 6.6 Hz, 3 H), 1.87 (m, 2 H), 3.07 (m, 1 H), 3.19 (m, 1 H), 3.68 (m, 1 H), 3.77 (d, J = 9.9 Hz, 1 H), 3.87 (m, 1 H), 3.92 (dd, J = 11.9, 3.2 Hz, 1 H), 4.11 (m, 1 H), 4.21 (dd, J = 11.9, 2.6 Hz, 1 H), 7.59 (m, 2 H), 7.68 (m, 4 H), 7.74 (d, J = 8.3 Hz, 2 H) |
| 30 | 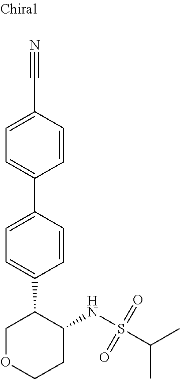 | N-[(3R,4R)-3- (4′-cyano biphenyl-4- yl)tetrahydro- 2H-pyran-4- yl]propane-2- sulfonamide | LCMS m/z 383.1 (M − 1). ¹H NMR (400 MHz, CHLOROFORM-d) δ 1.29 (d, J = 6.6 Hz, 3 H), 1.33 (d, J = 6.8 Hz, 3 H), 1.87 (m, 2 H), 3.08 (m, 1 H), 3.19 (m, 1 H), 3.70 (d, J = 10.3 Hz, 2 H), 3.88 (m, 1 H), 3.92 (dd, J = 12.0, 3.2 Hz, 1 H), 4.12 (m, 1 H), 4.22 (dd, J = 12.1, 2.5 Hz, 1 H), 7.59 (d, J = 8.2 Hz, 2 H), 7.69 (m, 4 H), 7.76 (d, J = 8.4 Hz, 2 H) |
| 31 | 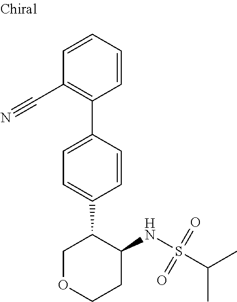 | N-[(3R,4S)-3- (2′-cyano biphenyl-4- yl)tetra hydro- 2H-pyran-4-yl] propane-2- sulfonamide | LCMS m/z 385 (M + 1). ¹H NMR (400 MHz, METHANOL-d₄) δ 0.72 (d, J = 6.9 Hz, 3 H), 1.05 (d, J = 6.9 Hz, 3 H), 1.80 (m, 1 H), 2.17 (m, 1 H), 2.43 (m, 1 H), 2.84 (ddd, J = 11.2, 11.2, 4.6 Hz, 1 H), 3.63 (m, 2 H), 3.72 (ddd, J = 11.3, 11.3, 4.5 Hz, 1 H), 3.93 (dd, J = 11.5, 4.5 Hz, 1 H), 4.03 (dd, J = 11.7, 4.5 Hz, 1 H), 7.53 (m, 6 H), 7.74 (ddd, apparent td, J = 7.7, 7.7, 1.2 Hz, 1 H), 7.84 (d, J = 7.9 Hz, 1 H) |
| 32 | 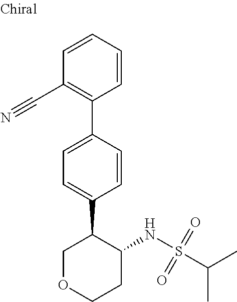 | N-[(3S,4R)-3- (2′-cyano biphenyl-4- yl)tetrahydro- 2H-pyran-4- yl]propane-2- sulfonamide | LCMS m/z 385 (M + 1). ¹H NMR (400 MHz, METHANOL-d₄) δ 0.72 (d, J = 6.8 Hz, 3 H), 1.05 (d, J = 6.8 Hz, 3 H), 1.79 (m, 1 H), 2.17 (m, 1 H), 2.43 (m, 1 H), 2.85 (ddd, apparent td, J = 11.3, 11.3, 4.6 Hz, 1 H), 3.64 (m, 2 H), 3.71 (m, 1 H), 3.93 (dd, J = 11.6, 4.6 Hz, 1 H), 4.03 (dd, J = 11.8, 4.6 Hz, 1 H), 7.52 (m, 6 H), 7.73 (ddd, apparent td, J = 7.7, 7.7, 1.1 Hz, 1 H), 7.83 (d, J = 7.6 Hz, 1 H) |
| 33 | 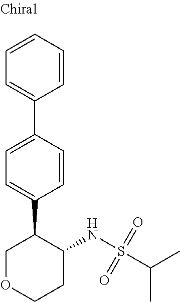 | N-[(3S,4R)-3- biphenyl-4- yltetrahydro- 2H-pyran-4- yl]propane-2- sulfonamide | LCMS m/z 358 (M − 1). ¹H NMR (400 MHz, CHLOROFORM-d) δ 0.76 (d, J = 6.6 Hz, 3 H), 1.13 (d, J = 6.8 Hz, 3 H), 1.78 (m, 1 H), 2.35 (m, 1 H), 2.53 (m, 1 H), 2.74 (ddd, apparent td, J = 11.0, 11.0, 4.5 Hz, 1 H), 3.53 (dd, apparent t, J = 11.5, 11.5 Hz, 1 H), 3.61 (ddd, apparent td, J = 12.2, 12.2, 2.0 Hz, 1 H), 3.71 (m, 1 H), 3.96 (d, J = 7.7 Hz, 1 H), 4.03 (dd, J = 11.9, 4.5 Hz, 1 H), 4.09 (m, 1 H ), 7.32 (d, J = 8.3 Hz, 2 H), 7.37 (m, 1 H), 7.46 (dd, apparent t, J = 7.6 Hz, 2 H), 7.57 (m, 4H) |
| 34 | 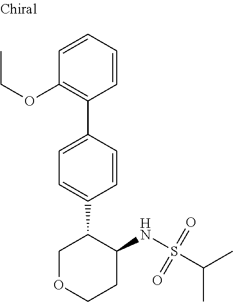 | N-[(3R,4S)-3- (2′-ethoxy biphenyl-4- yl)tetrahydro- 2H-pyran-4- yl]propane-2- sulfonamide | APCI m/z 401.6 (M − 1). ¹H NMR (400 MHz, CHLOROFORM-d) δ 0.71 (d, J = 6.8 Hz, 3 H), 1.13 (d, J = 6.8 Hz, 3 H), 1.35 (t, J = 6.9 Hz, 3 H), 1.81 (m, 1 H), 2.34 (m, 1 H), 2.50 (m, 1 H), 2.75 (ddd, apparent td, J = 11.1, 11.1, 4.6 Hz, 1 H), 3.55 (dd, apparent t, J = 11.4, 11.4 Hz, 1 H), 3.61 (ddd, apparent td, J = 12.2, 12.2, 1.7 Hz, 1 H), 3.67 (m, 1 H), 4.06 (q, J = 6.8 Hz, 2 H), 4.07 (m, 2 H), 4.46 (d, J = 8.3 Hz, 1 H), 6.98 (d, J = 8.3 Hz, 1 H), 7.02 (ddd, apparent td, J = 7.5, 7.5, 0.8 Hz, 1 H), 7.29 (m, 4 H), 7.55 (d, J = 8.3 Hz, 2 H) |
| 35 | 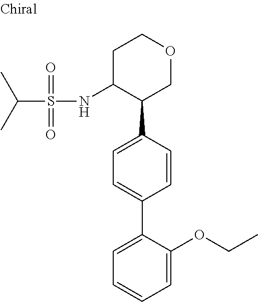 | N-[(3S,4R)-3- (2′- ethoxybiphenyl- 4- yl)tetrahydro- 2H-pyran-4- yl]propane-2- sulfonamide | LCMS m/z 402 (M − 1). ¹H NMR (400 MHz, CHLOROFORM-d) δ 0.74 (d, J = 6.8 Hz, 3 H), 1.14 (d, J = 6.8 Hz, 3 H), 1.35 (t, J = 7.1 Hz, 3 H), 1.78 (m, 1 H), 2.36 (m, 1 H), 2.53 (m, 1 H), 2.72 (ddd, apparent td, J = 11.1, 11.1, 4.6 Hz, 1 H), 3.55 (dd, apparent t, J = 11.4, 11.4 Hz, 1 H), 3.61 (ddd, apparent td, J = 12.2, 12.2, 1.8 Hz, 1 H), 3.69 (m, 1 H), 3.88 (d, J = 7.9 Hz, 1 H), 4.06 (q, J = 6.8 Hz, 2 H), 4.07 (m, 2 H), 6.98 (d, J = 8.1 Hz, 1 H), 7.02 (ddd, apparent td, J = 7.5, 7.5, 1.0 Hz, 1 H), 7.30 (m, 4 H), 7.55 (d, J = 8.1 Hz, 2 H) |
| 36 | 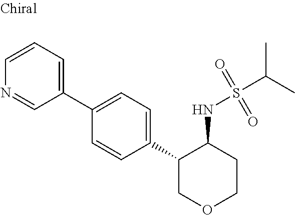 | N-[(3R,4S)-3- (4-pyridin-3- ylphenyl)tetra hydro-2H- pyran-4- yl]propane-2- sulfonamide | LCMS m/z 361.4 (M + 1). ¹H NMR (400 MHz, METHANOL-d₄) δ 0.72 (d, J = 6.6 Hz, 3 H), 1.06 (d, J = 6.8 Hz, 3 H), 1.79 (m, 1 H), 2.17 (m, 1 H), 2.52 (m, 1 H), 2.82 (ddd, apparent td, J = 11.2, 11.2, 4.6 Hz, 1 H), 3.58 (dd, apparent t, J = 11.3, 11.3 Hz, 1 H), 3.63 (m, 1 H), 3.71 (ddd, apparent td, J = 11.1, 11.1, 4.3 Hz, 1 H), 3.91 (dd, J = 11.5, 4.5 Hz, 1 H), 4.03 (dd, J = 11.7, 4.5 Hz, 1 H), 7.48 (d, J = 8.2 Hz, 2 H), 7.52 (dd, J = 8.0, 4.9 Hz, 1 H), 7.66 (d, J = 8.2 Hz, 2 H), 8.09 (ddd, apparent dt, J = 8.0, 1.9, 1.9 Hz, 1 H), 8.52 (dd, J = 4.9, 1.4 Hz, 1 H), 8.78 (d, J = 1.8 Hz, 1 H) |
| 37 | 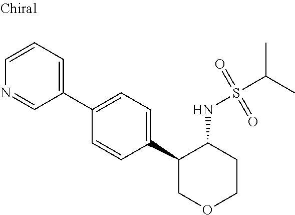 | N-[(3S,4R)-3- (4-pyridin-3- ylphenyl)tetra hydro-2H- pyran-4- yl]propane-2- sulfonamide | LCMS m/z 361.4 (M + 1). ¹H NMR (400 MHz, METHANOL-d₄) δ 0.72 (d, J = 6.6 Hz, 3 H), 1.06 (d, J = 6.8 Hz, 3 H), 1.79 (m, 1 H), 2.17 (m, 1 H), 2.52 (m, 1 H), 2.82 (ddd, apparent td, J = 11.2, 11.2, 4.6 Hz, 1 H), 3.59 (dd, apparent t, J = 11.3, 11.3 Hz, 1 H), 3.64 (m, 1 H), 3.71 (ddd, apparent td, J = 11.1, 11.1, 4.5 Hz, 1 H), 3.91 (dd, J = 11.4, 4.4 Hz, 1 H), 4.03 (dd, J = 11.7, 4.5 Hz, 1 H), 7.49 (d, J = 8.2 Hz, 2 H), 7.52 (dd, J = 8.0, 4.9 Hz, 1 H), 7.66 (d, J = 8.4 Hz, 2 H), 8.09 ((ddd, J = 8.3, 1.9, 1.7 Hz, 1 H)), 8.52 (dd, J = 4.9, 1.6 Hz, 1 H), 8.78 (d, J = 1.6 Hz, 1 H) |
| 38 | 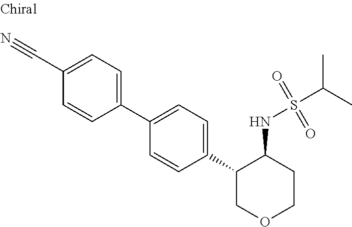 | N-[(3R,4S)-3- (4′- cyanobiphenyl- 4- yl)tetrahydro- 2H-pyran-4- yl]propane-2- sulfonamide | LCMS m/z 383.6 (M − 1). ¹H NMR (400 MHz, CHLOROFORM-d) δ 0.84 (d, J = 6.8 Hz, 3 H), 1.15 (d, J = 6.8 Hz, 3 H), 1.79 (m, 1 H), 2.34 (m, 1 H), 2.59 (m, 1 H), 2.78 (ddd, apparent td, J = 11.0, 11.0, 4.4 Hz, 1 H), 3.51 (dd, apparent t, J = 11.4, 11.4 Hz, 1 H), 3.61 (ddd, apparent td, J = 12.2, 12.2, 2.0 Hz, 1 H), 3.75 (m, 1 H), 3.92 (d, J = 7.9 Hz, 1 H), 4.01 (dd, J = 11.7, 4.5 Hz, 1 H), 4.09 (dd, J = 12.0, 4.4 Hz, 1 H), 7.38 (d, J = 8.3 Hz, 2 H), 7.59 (d, J = 8.3 Hz, 2 H), 7.66 (d, J = 8.5 Hz, 2 H), 7.74 (d, J = 8.3 Hz, 2 H) |
| 39 | 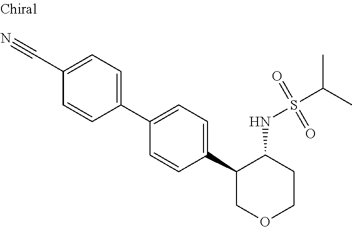 | N-[(3S,4R)-3- (4′- cyanobiphenyl- 4- yl)tetrahydro- 2H-pyran-4- yl]propane-2- sulfonamide | LCMS m/z 383.1 (M − 1). ¹H NMR (400 MHz, CHLOROFORM-d) δ 0.84 (d, J = 6.6 Hz, 3 H), 1.15 (d, J = 6.8 Hz, 3 H), 1.79 (m, 1 H), 2.34 (br d, J = 11.8 Hz, 1 H), 2.60 (m, 1 H), 2.78 (ddd, apparent td, J = 10.9, 10.9, 4.4 Hz, 1 H), 3.52 (dd, apparent t, J = 11.4, 11.4 Hz, 1 H), 3.61 (dd, apparent t, J = 11.6, 11.6 Hz, 1 H), 3.74 (m, 1 H), 3.93 (d, J = 7.7 Hz, 1 H), 4.01 (dd, J = 11.6, 4.1 Hz, 1 H), 4.09 (br d, J = 11.4 Hz, 1 H), 7.38 (d, J = 7.9 Hz, 2 H), 7.59 (d, J = 8.1 Hz, 2 H), 7.66 (d, J = 8.1 Hz, 2 H), 7.74 (d, J = 8.3 Hz, 2 H) |
| 40 | 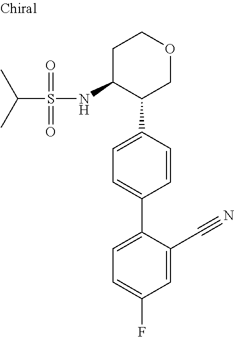 | N-[(3R,4S)-3- (2′-cyano-4′- fluorobiphenyl- 4- yl)tetrahydro- 2H-pyran-4- yl]propane-2- sulfonamide | LCMS m/z 403 (M + 1). ¹H NMR (400 MHz, CHLOROFORM-d) δ 0.77 (d, J = 6.8 Hz, 3 H), 1.12 (d, J = 6.8 Hz, 3 H), 1. 80 (m, 1 H), 2.31 (m, 1 H), 2.49 (m, 1 H), 2.79 (ddd, J = 11.1, 11.1, 4.6 Hz, 1 H), 3.52 (dd, apparent t, J = 11.5, 11.5 Hz, 1 H), 3.60 (m, 1 H), 3.70 (m, 1 H), 4.03 (m, 2 H), 4.52 (d, J = 8.7 Hz, 1 H), 7.38 (m, 3 H), 7.46 (m, 2 H), 7.50 (d, J = 8.1 Hz, 2 H) |
| 41 | 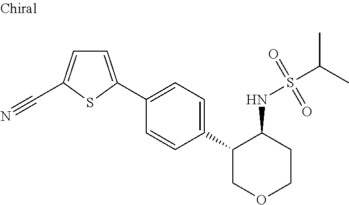 | N-{(3R,4S)-3- [4-(5-cyano-2- thienyl)phenyl] tetrahydro-2H- pyran-4- yl}propane-2- sulfonamide | LCMS m/z 389.2 (M − 1). ¹H NMR (400 MHz, CHLOROFORM-d) δ 0.88 (d, J = 6.8 Hz, 3 H), 1.16 (d, J = 6.8 Hz, 3 H), 1.77 (m, 1 H), 2.33 (m, 1 H), 2.63 (m, 1 H), 2.76 (ddd, J = 10.9, 10.9, 4.4 Hz, 1 H), 3.48 (dd, apparent t, J = 11.3, 11.3 Hz, 1 H), 3.60 (ddd, apparent td, J = 12.2, 12.2, 2.0 Hz, 1 H ), 3.74 (m, 1 H), 3.85 (d, J = 7.9 Hz, 1 H), 3.99 (dd, J = 11.7, 4.5 Hz, 1 H), 4.09 (dd, J = 11.8, 4.1 Hz, 1 H), 7.28 (d, J = 4.2 Hz, 1 H), 7.33 (d, J = 8.3 Hz, 2 H), 7.59 (d, J = 8.1 Hz, 2 H), 7.60 (d, J = 3.7 Hz, 1 H) |
| 42 | 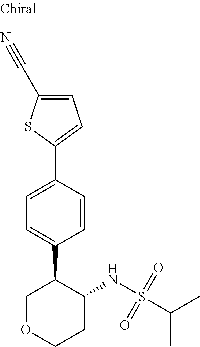 | N-{(3S,4R)-3- [4-(5-cyano-2- thienyl)phenyl] tetrahydro-2H- pyran-4- yl}propane-2- sulfonamide | LCMS m/z 389.0 (M − 1). ¹H NMR (400 MHz, CHLOROFORM-d) δ 0.88 (d, J = 6.8 Hz, 3 H), 1.16 (d, J = 6.8 Hz, 3 H), 1.77 (m, 1 H), 2.33 (m, 1 H), 2.62 (m, 1 H), 2.76 (ddd, apparent td, J = 10.9, 10.9, 4.5 Hz, 1 H), 3.48 (m, 1 H), 3.60 (ddd, apparent td, J = 12.2, 12.2, 2.0 Hz, 1 H), 3.74 (m, 1 H), 3.87 (d, J = 8.1 Hz, 1 H), 3.99 (dd, J = 11.7, 4.3 Hz, 1 H), 4.09 (dd, J = 11.9, 4.3 Hz, 1 H), 7.28 (d, J = 4.3 Hz, 1 H), 7.33 (d, J = 8.3 Hz, 2 H), 7.60 (d, J = 8.3 Hz, 2 H), 7.61 (d, J = 3.9 Hz, 1 H) |
| 43 | 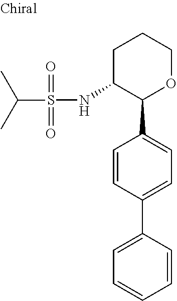 | N-[(2S,3R)-2- biphenyl-4- yltetrahydro- 2H-pyran-3- yl]propane-2- sulfonamide | MS (APCI) m/z 357.6 (M − 1). ¹H NMR (400 MHz, CHLOROFORM- d) δ 0.73 (d, J = 6.6 Hz, 3 H), 1.06 (d, J = 6.8 Hz, 3 H), 1.64 (m, 1 H), 1.79 (br d, J = 13.7 Hz, 1 H), 1.93 (m, 1 H), 2.31 (m, 1 H), 2.54 (m, 1 H), 3.46 (m, 1 H), 3.54 (ddd, apparent td, J = 11.9, 11.9, 2.4 Hz, 1 H), 3.84 (d, J = 8.9 Hz, 1 H), 4.02 (d, J = 9.7 Hz, 1 H), 4.10 (m, 1 H), 7.37 (m, 1 H), 7.46 (m, 4 H), 7.59 (m, 4 H) |
| 44 | 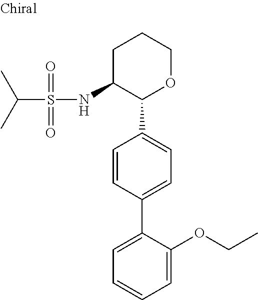 | N-[(2R,3S)-2- (2′-ethoxy biphenyl-4- yl)tetrahydro- 2H-pyran-3- yl]propane-2- sulfonamide | MS (APCI) m/z 402.3 (M − 1). ¹H NMR (500 MHz, CHLOROFORM- d) δ 0.72 (d, J = 6.6 Hz, 3 H), 1.07 (d, J = 7.1 Hz, 3 H), 1.33 (t, J = 7.1 Hz, 3 H), 1.63 (m, 1 H), 1.78 (br d, J = 13.9 Hz, 1 H), 1.94 (m, 1 H), 2.32 (m, 1 H), 2.55 (m, 1 H), 3.47 (m, 1 H), 3.54 (ddd, apparent td, J = 11.8, 11.8, 2.2 Hz, 1 H), 3.86 (d, J = 9.0 Hz, 1 H), 4.01 (d, J = 9.8 Hz, 1 H), 4.05 (q, J = 7.1 Hz, 2 H), 4.10 (m, 1 H), 6.98 (d, J = 8.1 Hz, 1 H), 7.02 (ddd, apparent td, J = 7.6, 7.6, 0.8 Hz, 1 H), 7.26 (m, 1 H), 7.31 (m, 1 H), 7.42 (d, J = 8.3 Hz, 2 H), 7.56 (d, J = 8.1 Hz, 2 H) |
| 45 | 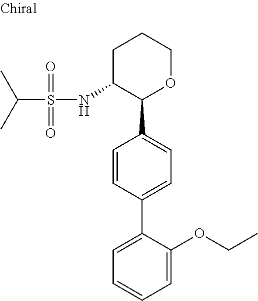 | N-[(2S,3R)-2- (2′- ethoxybiphenyl- 4- yl)tetrahydro- 2H-pyran-3- yl]propane-2- sulfonamide | MS (APCI) m/z 401.7 (M − 1). ¹H NMR (500 MHz, CHLOROFORM- d) δ 0.72 (d, J = 6.8 Hz, 3 H), 1.07 (d, J = 7.1 Hz, 3 H), 1.33 (t, J = 7.0 Hz, 3 H), 1.62 (m, 1 H), 1.79 (br d, J = 13.7 Hz, 1 H), 1.94 (m, 1 H), 2.32 (m, 1 H), 2.56 (m, 1 H), 3.48 (m, 1 H), 3.54 (ddd, apparent td, J = 11.9, 11.9, 2.3 Hz, 1 H), 3.77 (d, J = 8.8 Hz, 1 H), 4.00 (d, J = 9.8 Hz, 1 H), 4.04 (q, J = 6.9 Hz, 2 H), 4.10 (m, 1 H), 6.98 (d, J = 8.3 Hz, 1 H), 7.02 (ddd, apparent td, J = 7.3, 7.3, 1.0 Hz, 1 H), 7.26 (m, 1 H), 7.31 (m, 1 H), 7.42 (d, J = 8.1 Hz, 2 H), 7.57 (d, J = 8.1 Hz, 2 H) |
| 46 | 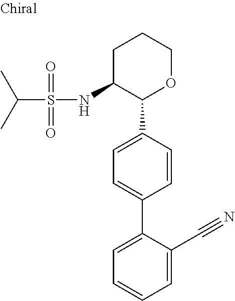 | N-[(2R,3S)-2- (2′-cyano biphenyl-4- yl)tetrahydro- 2H-pyran-3- yl]propane-2- sulfonamide | MS (APCI) m/z 382.8 (M − 1). ¹H NMR (500 MHz, CHLOROFORM- d) δ 0.81 (d, J = 6.6 Hz, 3 H), 1.08 (d, J = 6.8 Hz, 3 H), 1.64 (m, 1 H), 1.80 (m, 1 H), 1.94 (m, 1 H), 2.33 (m, 1 H), 2.55 (br d, J = 12.9 Hz, 1 H), 3.51 (m, 2 H), 3.81 (d, J = 9.3 Hz, 1 H), 4.05 (br d, J = 9.8 Hz, 1 H), 4.10 (m, 1 H), 7.47 (m, 2 H), 7.55 (d, J = 8.1 Hz, 2 H), 7.58 (d, J = 7.8 Hz, 2 H), 7.66 (dd, apparent t, J = 7.6, 7.6 Hz, 1 H), 7.78 (d, J = 7.6 Hz, 1 H) |
| 47 | 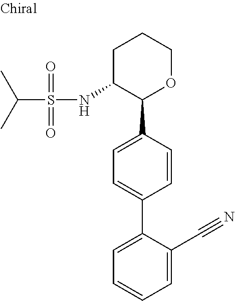 | N-[(2S,3R)-2- (2′- cyanobiphenyl- 4- yl)tetrahydro- 2H-pyran-3- yl]propane-2- sulfonamide | MS (APCI) m/z 382.8 (M − 1). ¹H NMR (400 MHz, CHLOROFORM- d) δ 0.80 (d, J = 6.8 Hz, 3 H), 1.08 (d, J = 6.8 Hz, 3 H), 1.64 (m, 1 H), 1.80 (br d, J = 13.7 Hz, 1 H), 1.95 (m, 1 H), 2.32 (m, 1 H), 2.54 (m, 1 H), 3.51 (m, 2 H), 3.89 (d, J = 9.4 Hz, 1 H), 4.05 (d, J = 9.8 Hz, 1 H), 4.10 (m, 1 H), 7.47 (m, 2 H), 7.54 (d, J = 8.4 Hz, 2 H), 7.58 (d, J = 8.4 Hz, 2 H), 7.66 (ddd, apparent td, J = 7.7, 7.7, 1.3 Hz 1 H), 7.78 (dd, J = 8.0, 1.4 Hz, 1 H) |
| 48 | 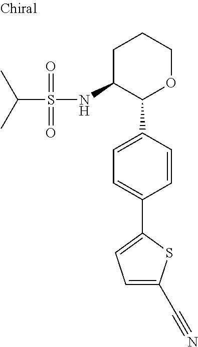 | N-{(2R,3S)-2- [4-(5-cyano-2- thienyl)phenyl] tetrahydro-2H- pyran-3- yl}propane-2- sulfonamide | LCMS m/z 388.7 (M − 1). ¹H NMR (500 MHz, CHLOROFORM-d) δ 0.84 (d, J = 6.6 Hz, 3 H), 1.09 (d, J = 6.8 Hz, 3 H), 1.64 (m, 1 H), 1.80 (br d, J = 13.9 Hz, 1 H), 1.93 (m, 1 H), 2.43 (m, 1 H), 2.51 (m, 1 H), 3.43 (m, 1 H), 3.53 (ddd, apparent td, J = 11.9, 11.9, 2.3 Hz, 1 H), 3.77 (d, J = 9.0 Hz, 1 H), 4.03 (d, J = 9.5 Hz, 1 H), 4.09 (m, 1 H), 7.29 (d, J = 3.9 Hz, 1 H), 7.48 (d, J = 8.3 Hz, 2 H), 7.61 (m, 3 H) |
| 49 | 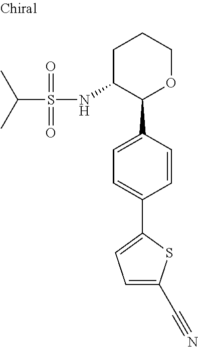 | N-{(2S,3R)-2- [4-(5-cyano-2- thienyl)phenyl] tetrahydro-2H- pyran-3- yl}propane-2- sulfonamide | MS (APCI) m/z 388.7 (M − 1). ¹H NMR (400 MHz, CHLOROFORM- d) δ 0.84 (d, J = 6.6 Hz, 3 H), 1.09 (d, J = 6.8 Hz, 3 H), 1.64 (m, 1 H), 1.80 (br d, J = 13.7 Hz, 1 H), 1.92 (m, 1 H), 2.43 (m, 1 H), 2.51 (m, 1 H), 3.43 (m, 1 H), 3.53 (ddd apparent td, J = 11.9, 11.9, 2.4 Hz, 1 H), 3.78 (d, J = 9.2 Hz, 1 H), 4.03 (d, J = 9.8 Hz, 1 H), 4.09 (m, 1 H), 7.29 (d, J = 3.9 Hz, 1 H), 7.48 (d, J = 8.2 Hz, 2 H), 7.61 (m, 3 H) |
| 50 | 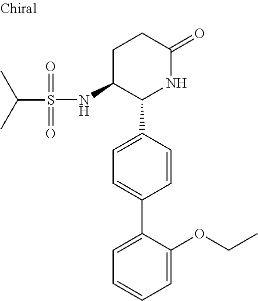 | N-[(2R,3S)-2- (2′- ethoxybiphenyl- 4-yl)-6- oxopiperidin- 3-yl]propane- 2-sulfonamide | LCMS m/z 417.0 (M + 1). ¹H NMR (500 MHz, CHLOROFORM- d) δ 1.19 (d, J = 6.6 Hz, 3 H), 1.29 (d, J = 6.8 Hz, 3 H), 1.36 (t, J = 7.0 Hz, 3 H), 1.84 (m, 1 H), 2.22 (m, 1 H), 2.61 (m, 1 H), 2.75 (m, 1 H), 2.92 (m, 1 H), 3.81 (m, 1 H), 4.06 (q, J = 7.1 Hz, 2 H), 4.74 (br s, 1 H), 5.90 (d, J = 9.0 Hz, 1 H), 6.63 (br s, 1 H), 6.99 (d, J = 8.1 Hz, 1 H), 7.03 (dd, apparent t, J = 7.4 Hz, 1 H), 7.31 (d, J = 7.8 Hz, 1 H), 7.31 (m, 1 H 8 Hz, 1 H), 7.32 (m, 1 H), 7.38 (d, J = 8.1 Hz, 2 H), 7.60 (d, J = 8.1 Hz, 2H) |
| 51 | 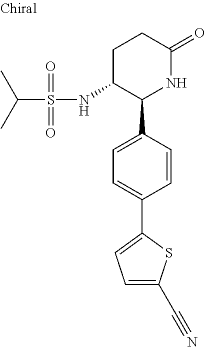 | N-{(2S,3R)-2- [4-(5-cyano-2- thienyl)phenyl]- 6- oxopiperidin- 3-yl}propane- 2-sulfonamide | LCMS m/z 404.0 (M + 1). ¹H NMR (500 MHz, CHLOROFORM- d) δ 1.36 (d, J = 6.8 Hz, 3 H), 1.37 (d, J = 6.8 Hz, 3 H), 1.79 (m, 1 H), 2.00 (m, 1 H), 2.56 (m, 1 H), 2.81 (m, 1 H), 3.14 (m, 1 H), 3.83 (m, 1 H), 4.94 (br s, 1 H), 6.87 (d, J = 9.3 Hz, 1 H), 7.15 (d, J = 3.4 Hz, 1 H), 7.29 (d, J = 4.1 Hz, 1 H), 7.43 (d, J = 8.3 Hz, 2 H), 7.61 (m, 3 H) |
| 52 | 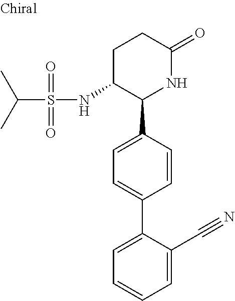 | N-[(2S,3R)-2- (2′- cyanobiphenyl- 4-yl)-6- oxopiperidin- 3-yl]propane- 2-sulfonamide | LCMS m/z 396.1 (M − 1). ¹H NMR (400 MHz, CHLOROFORM-d) δ 1.25 (d, J = 6.8 Hz, 3 H), 1.31 (d, J = 6.8 Hz, 3 H), 1.86 (m, 1 H), 2.17 (m, 1 H), 2.62 (m, 1 H), 2.75 (m, 1 H), 2.97 (m, 1 H), 3.84 (m, 1 H), 4.82 (dd, apparent t, J = 3.7, 3.7 Hz, 1 H), 5.98 (d, J = 8.8 Hz, 1 H), 6.70 (br d, J = 2.1 Hz, 1 H), 7.49 (m, 4 H), 7.60 (d, J = 8.2 Hz, 2 H), 7.68 (ddd, apparent td, J = 7.7, 7.7, 1.3 Hz, 1 H), 7.79 (br d, J = 7.8 Hz, 1 H) |
| 53 | 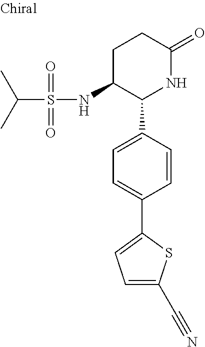 | N-{(2R,3S)-2- [4-(5-cyano-2- thienyl)phenyl]- 6- oxopiperidin- 3-yl}propane- 2-sulfonamide | LCMS m/z 404.1 (M + 1). ¹H NMR (400 MHz, CHLOROFORM- d) δ 1.37 (d, J = 6.8 Hz, 3 H), 1.38 (d, J = 6.8 Hz, 3 H), 1.78 (m, 1 H), 1.98 (m, 1 H), 2.54 (m, 1 H), 2.82 (m, 1 H), 3.16 (m, 1 H), 3.83 (m, 1 H), 4.97 (br s, 1 H), 7.09 (d, J = 9.4 Hz, 1 H), 7.26 (d, J = 3.7 Hz, 1 H), 7.29 (d, J = 3.9 Hz, 1 H), 7.43 (d, J = 8.2 Hz, 2 H), 7.61 (m, 3 H) |
| 54 | 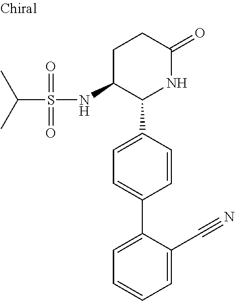 | N-[(2R,3S)-2- (2′- cyanobiphenyl- 4-yl)-6- oxopiperidin- 3-yl]propane- 2-sulfonamide | LCMS m/z 398.1 (M + 1). ¹H NMR (400 MHz, CHLOROFORM- d) δ 1.31 (d, J = 6.8 Hz, 3 H), 1.35 (d, J = 6.8 Hz, 3 H), 1.82 (m, 1 H), 2.11 (m, 1 H), 2.58 (m, 1 H), 2.81 (m, 1 H), 3.06 (m, 1 H), 3.85 (m, 1 H), 4.92 (dd, apparent t, J = 3.2, 3.2 Hz, 1 H), 6.73 (d, J = 9.2 Hz, 1 H), 7.07 (br d, J = 3.1 Hz, 1 H), 7.48 (m, 4 H), 7.59 (d, J = 8.2 Hz, 2 H), 7.67 (ddd, apparent td, J = 7.7, 7.7, 1.2 Hz, 1 H), 7.78 (d, J = 7.8 Hz, 1 H) |
| 55 | 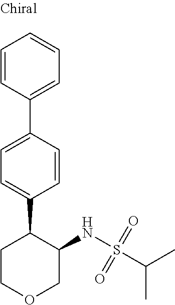 | N-[(3R,4S)-4- biphenyl-4- yltetrahydro- 2H-pyran-3- yl]propane-2- sulfonamide | LCMS m/z 360.1 (M + 1). ¹H NMR (500 MHz, CHLOROFORM- d) δ 0.83 (d, J = 6.7 Hz, 3 H), 1.04 (d, J = 7.0 Hz, 3 H), 1.81 (br d, J = 13.5 Hz, 1 H), 2.19 (m, 2 H), 3.14 (m, 1 H), 3.59 (dd, apparent t, J = 11.7, 11.7 Hz, 1 H), 3.75 (m, 2 H), 4.16 (m, 2 H), 4.55 (d, J = 9.4 Hz, 1 H), 7.33 (d, J = 8.1 Hz, 2 H), 7.37 (t, J = 7.3 Hz, 1 H), 7.46 (dd, apparent t, J = 7.6, 7.6 Hz, 2 H), 7.58 (m, 4H) |
| 56 | 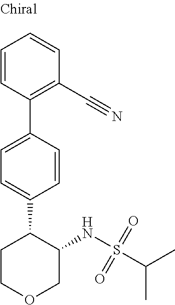 | N-[(3S,4R)-4- (2′- cyanobiphenyl- 4- yl)tetrahydro- 2H-pyran-3- yl]propane-2- sulfonamide | LCMS m/z 385.6 (M + 1). ¹H NMR (500 MHz, CHLOROFORM- d) δ 0.89 (d, J = 6.7 Hz, 3 H), 1.07 (d, J = 7.0 Hz, 3 H), 1.82 (br d, J = 12.4 Hz, 1 H), 2.18 (m, 1 H), 2.26 (m, 1 H), 3.16 (ddd, apparent dt, J = 13.1, 3.3, 3.3 Hz, 1 H), 3.59 (ddd, apparent dt, J = 11.9, 11.9, 2.0 Hz, 1 H), 3.76 (dd, J = 11.7, 1.0 Hz, 1 H), 3.81 (br d, J = 8.8 Hz, 1 H), 4.19 (m, 2 H), 4.58 (d, J = 9.8 Hz, 1 H), 7.40 (d, J = 8.1 Hz, 2 H), 7.48 (m, 2 H), 7.57 (d, J = 8.2 Hz, 2 H), 7.66 (ddd, apparent td, J = 7.7, 7.7, 1.1 Hz, 1 H), 7.78 (d, J = 7.6 Hz, 1 H) |
| 57 | 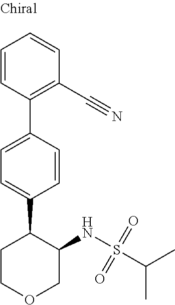 | N-[(3R,4S)-4- (2′-cyano biphenyl-4- yl)tetrahydro- 2H-pyran-3- yl]propane-2- sulfonamide | LCMS m/z 385.1 (M + 1). ¹H NMR (500 MHz, CHLOROFORM- d) δ 0.89 (d, J = 6.7 Hz, 3 H), 1.07 (d, J = 6.8 Hz, 3 H), 1.82 (br d, J = 13.7 Hz, 1 H), 2.23 (m, 2 H), 3.16 (ddd, apparent dt, J = 13.0, 3.4, 3.4 Hz, 1 H), 3.59 (ddd, apparent td, J = 12.0, 12.0, 2.2 Hz, 1 H), 3.76 (dd, J = 11.7, 1.3 Hz, 1 H), 3.82 (d, J = 9.7 Hz, 1 H), 4.20 (m, 2 H), 4.57 (d, J = 9.8 Hz, 1 H), 7.40 (d, J = 8.2 Hz, 2 H), 7.47 (m, 2 H), 7.57 (d, J = 8.2 Hz, 2 H), 7.66 (ddd, apparent td, J = 7.7, 7.7, 1.2 Hz, 1 H), 7.78 (d, J = 7.5 Hz, 1 H) |
| 58 | 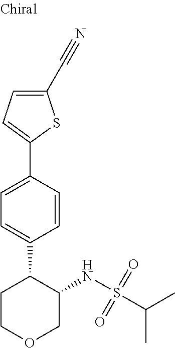 | N-{(3S,4R)-4- [4-(5-cyano-2- thienyl)phenyl] tetrahydro-2H- pyran-3- yl}propane-2- sulfonamide | LCMS m/z 389.6 (M − 1). ¹H NMR (500 MHz, CHLOROFORM-d) δ 0.92 (d, J = 6.7 Hz, 3 H), 1.09 (d, J = 6.8 Hz, 3 H), 1.79 (br d, J = 13.7 Hz, 1 H), 2.16 (m, 1 H), 2.38 (m, 1 H), 3.12 (ddd, apparent dt, J = 13.1, 3.1, 3.1 Hz, 1 H), 3.58 (ddd, apparent td, J = 11.9, 11.9, 2.0 Hz, 1 H), 3.74 (m, 2 H), 4.13 (d, J = 10.4 Hz, 1 H), 4.18 (dd, J = 11.6, 4.3 Hz, 1 H), 4.57 (d, J = 9.6 Hz, 1 H), 7.28 (m, 1 H), 7.34 (d, J = 8.2 Hz, 2 H), 7.60 (m, 3 H) |
| 59 | 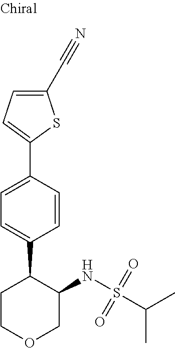 | N-{(3R,4S)-4- [4-(5-cyano-2- thienyl)phenyl] tetrahydro-2H- pyran-3- yl}propane-2- sulfonamide | LCMS m/z 389.0 (M − 1). ¹H NMR (500 MHz, CHLOROFORM-d) δ 0.91 (d, J = 6.8 Hz, 3 H), 1.08 (d, J = 6.8 Hz, 3 H), 1.77 (br d, J = 13.5 Hz, 1 H), 2.17 (m, 1 H), 2.38 (m, 1 H), 3.12 (ddd, apparent dt, J = 13.1, 3.3, 3.3 Hz, 1 H), 3.58 (ddd, apparent dt, J = 11.9, 11.9, 1.8 Hz, 1 H), 3.74 (m, 2 H), 4.14 (m, 1 H), 4.18 (dd, J = 11.5, 4.3 Hz, 1 H), 4.64 (d, J = 9.6 Hz, 1 H), 7.28 (m, 1 H), 7.33 (d, J = 8.3 Hz, 2 H), 7.60 (m, 3 H) |
| 60 | 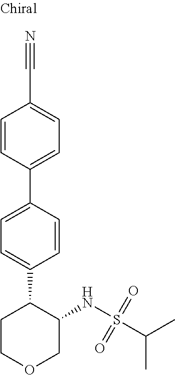 | N-[(3S,4R)-4- (4′- cyanobiphenyl- 4- yl)tetrahydro- 2H-pyran-3- yl]propane-2- sulfonamide | LCMS m/z 383.6 (M − 1). ¹H NMR (500 MHz, CHLOROFORM-d) δ 0.89 (d, J = 6.7 Hz, 3 H), 1.07 (d, J = 7.0 Hz, 3 H), 1.81 (br d, J = 13.7 Hz, 1 H), 2.19 (m, 1 H), 2.33 (m, 1 H), 3.15 (ddd, apparent dt, J = 13.0, 3.2, 3.2 Hz, 1 H), 3.59 (ddd, apparent td, J = 11.8, 11.8, 2.1 Hz, 1 H), 3.76 (d, J = 11.7 Hz, 1 H), 3.78 (br d, J = 9.4 Hz, 1 H), 4.15 (d, J = 10.5 Hz, 1 H), 4.19 (dd, J = 11.6, 4.1 Hz, 1 H), 4.56 (d, J = 9.5 Hz, 1 H), 7.39 (d, J = 8.3 Hz, 2 H), 7.60 (d, J = 8.3 Hz, 2 H), 7.67 (d, J = 8.3 Hz, 2 H), 7.74 (d, J = 8.3 Hz, 2 H) |
| 61 | 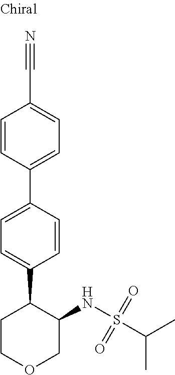 | N-[(3R,4S)-4- (4′- cyanobiphenyl- 4- yl)tetrahydro- 2H-pyran-3- yl]propane-2- sulfonamide | LCMS m/z 383.1 (M − 1). ¹H NMR (500 MHz, CHLOROFORM-d) δ 0.90 (d, J = 6.7 Hz, 3 H), 1.07 (d, J = 7.0 Hz, 3 H), 1.80 (br d, J = 12.0 Hz, 1 H), 2.18 (m, 1 H), 2.33 (m, 1 H), 3.15 (ddd, apparent dt, J = 13.0, 3.4, 3.4 Hz, 1 H), 3.59 (ddd, apparent td, J = 11.9, 11.9, 2.2 Hz, 1 H), 3.75 (dd, J = 11.8, 1.3 Hz, 1 H), 3.78 (br d, J = 10.1 Hz, 1 H), 4.16 (m, 1 H), 4.19 (dd, J = 11.5, 4.2 Hz, 1 H), 4.55 (d, J = 9.4 Hz, 1 H), 7.39 (d, J = 8.2 Hz, 2 H), 7.60 (d, J = 8.3 Hz, 2 H), 7.67 (d, J = 8.3 Hz, 2 H), 7.74 (d, J = 8.3 Hz, 2 H) |
| 62 | 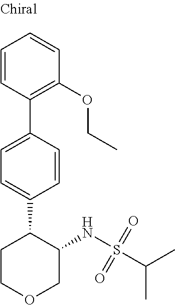 | N- [(3S,4R)-4-(2′- ethoxybiphenyl- 4- yl)tetrahydro- 2H-pyran-3- yl]propane-2- sulfonamide | LCMS m/z 402.7 (M − 1). ¹H NMR (500 MHz, CHLOROFORM-d) δ 0.81 (d, J = 6.7 Hz, 3 H), 1.04 (d, J = 6.8 Hz, 3 H), 1.36 (t, J = 7.0 Hz, 3 H), 1.81 (br d, J = 12.1 Hz, 1 H), 2.19 (m, 2 H), 3.13 (ddd, apparent dt, J = 13.2, 3.5, 3.5 Hz, 1 H), 3.58 (ddd, apparent td, J = 11.9, 11.9, 2.1 Hz, 1 H), 3.75 (dd, J = 11.7, 1.5 Hz, 1 H), 3.78 (br d, J = 9.3 Hz, 1 H), 4.07 (q, J = 7.0 Hz, 2 H), 4.18 (m, 2 H), 4.54 (d, J = 9.5 Hz, 1 H), 6.99 (d, J = 8.2 Hz, 1 H), 7.03 (ddd, apparent td, J = 7.5, 7.5, 1.0 Hz, 1 H), 7.28 (m, 3 H), 7.31 (m, 1 H), 7.56 (d, J = 8.3 Hz, 2 H) |
| 63 | 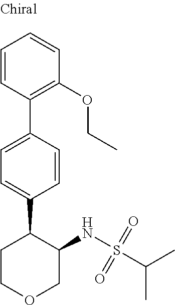 | N-[(3R,4S)-4- (2′-ethoxy biphenyl-4- yl)tetrahydro- 2H-pyran-3- yl]propane-2- sulfonamide | LCMS m/z 404.1 (M + 1). ¹H NMR (500 MHz, CHLOROFORM- d) δ 0.81 (d, J = 6.7 Hz, 3 H), 1.04 (d, J = 7.0 Hz, 3 H), 1.35 (t, J = 7.0 Hz, 3 H), 1.82 ( br d, J = 11.8 Hz, 1 H), 2.18 (m, 2 H), 3.13 (ddd, apparent dt, J = 13.1, 3.6, 3.6 Hz, 1 H), 3.58 (ddd, apparent td, J = 11.9, 11.9, 2.2 Hz, 1 H), 3.75 (dd, J = 11.7, 1.5 Hz, 1 H), 3.78 (br d, J = 9.0 Hz, 1 H), 4.07 (q, J = 7.0 Hz, 2 H), 4.18 (m, 2 H), 4.53 (d, J = 9.4 Hz, 1 H), 6.99 (d, J = 8.2 Hz, 1 H), 7.03 (ddd, apparent td, J = 7.4, 7.4, 1.0 Hz, 1 H), 7.28 (m, 3 H), 7.31 (m, 1 H), 7.56 (d, J = 8.2 Hz, 2 H) |
| 64 | 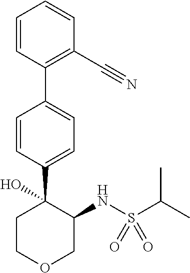 | N-[(3S,4R)-4- (2′- cyanobiphenyl- 4-yl)-4- hydroxytetrahydro- 2H-pyran- 3-yl]propane-2- sulfonamide | LCMS m/z 399.6 (M − 1). ¹H NMR (500 MHz, METHANOL-d₄) δ 0.77 (d, J = 6.6 Hz, 3 H), 0.99 (d, J = 6.8 Hz, 3 H), 1.69 (br d, J = 13.9 Hz, 1 H), 2.18 (m, 1 H), 2.77 (m, 1 H), 3.52 (brs, 1 H), 3.80 (br d, J = 11.5 Hz, 1 H), 3.92 (dd, J = 11.0, 5.1 Hz, 1 H), 3.98 (ddd, apparent td, J = 11.6, 11.6, 2.1 Hz, 1 H), 4.23 (dd, J = 11.3,1.6 Hz, 1 H), 7.54 (m, 2 H), 7.59 (d, J = 8.3 Hz, 2 H), 7.71 (d, J = 8.3 Hz, 2 H), 7.74 (m, 1 H), 7.83 (br d, J = 7.8 Hz, 1 H). Retention time (HPLC)³ 3.80 min |
| 65 | 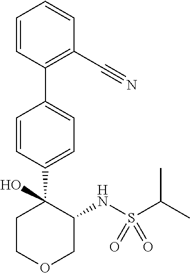 | N-[(3R,4S)-4- (2′- cyanobiphenyl- 4-yl)-4- hydroxytetrahydro- 2H- pyran-3- yl]propane-2- sulfonamide | LCMS m/z 399.6 (M − 1) ¹H NMR (500 MHz, METHANOL-d₄) δ 0.77 (d, J = 6.6 Hz, 3H), 0.99 (d, J = 6.8 Hz, 3H), 1.70 (br d, J = 14.0 Hz, 1H), 2.19 (septet, J = 6.8 Hz, 1H), 2.77 (m, 1H), 3.52 (brs, 1H), 3.80 (br d, J = 11.5 Hz, 1H), 3.92 (br dd, J = 10.8, 5.0 Hz, 1H), 3.98 (m, 1H), 4.24 (dd, J = 11.2, 1.7 Hz, 1H), 7.54 (m, 1H), 7.56 (br d, J = 8 Hz, 1H), 7.59 (d, J = 8.5 Hz, 2H), 7.71 (d, J = 8.5 Hz, 2H), 7.74 (ddd, J = 7.8, 7.8, 1.5 Hz, 1H), 7.84 (br d, J = 7.8 Hz, 1H). Retention time (HPLC)³ 4.80 min |
| 66 | 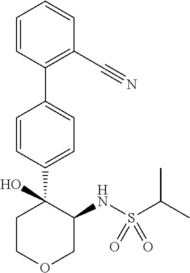 | N-[(3S,4S)-4- (2′- cyanobiphenyl- 4-yl)-4- hydroxytetrahydro- 2H- pyran-3- yl]propane-2- sulfonamide | LCMS m/z 399.6 (M − 1). ¹H NMR (500 MHz, METHANOL-d₄) δ 0.70 (d, J = 6.7 Hz, 3H), 1.00 (d, J = 6.8 Hz, 3H), 1.75 (br d, J = 14.3 Hz, 1H), 2.26 (septet, J = 6.8 Hz, 1H), 2.40 (m, 1H), 3.73 (m, 2H), 3.81 (br dd, J = 11.4, 4.6 Hz, 1H), 3.87-3.92 (m, 2H), 7.54 (ddd, J = 7.7, 7.7, 1.2 Hz, 1H), 7.57 (br d, J = 7.9 Hz, 1H), 7.60 (d, J = 8.5 Hz, 2H), 7.74 (m, 1H), 7.76 (d, J = 8.5 Hz, 2H), 7.84 (dd, J = 7.7 Hz, 0.9 Hz, 1H). Retention time (HPLC)⁴ 5.05 min |
| 67 | 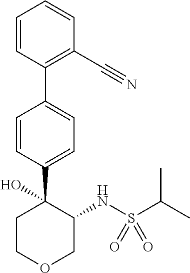 | N-[(3R,4R)-4- (2′- cyanobiphenyl- 4-yl)-4- hydroxytetrahydro- 2H- pyran-3- yl]propane-2- sulfonamide | LCMS m/z 399.6 (M − 1). ¹H NMR (400 MHz, METHANOL-d₄) δ 0.70 (d, J = 6.7 Hz, 3H), 1.00 (d, J = 6.8 Hz, 3H), 1.75 (br d, J = 14.2 Hz, 1H), 2.26 (septet, J = 6.8 Hz, 1H), 2.40 (m, 1H), 3.72 (m, 2H), 3.81 (br d, J = 11.2, 4.5 Hz, 1H), 3.87-3.94 (m, 2H), 7.51-7.58 (m, 2H), 7.60 (d, J = 8.7 Hz, 2H), 7.74 (m, 1H), 7.76 (d, J = 8.7 Hz, 2H), 7.84 (br d, J = 7.8 Hz, 1H). Retention time (HPLC)⁴ 7.42 min |
| 68 | 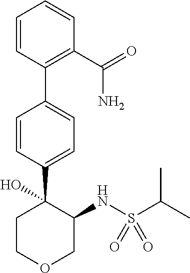 | 4′-{(3S,4R)-4- hydroxy-3- [(isopropylsulfonyl) amino] tetrahydro-2H- pyran-4- yl}biphenyl-2- carboxamide | LCMS m/z 419.7 (M + 1). ¹H NMR (400 MHz, METHANOL-d₄) δ 0.81 (d, J = 6.6 Hz, 3H), 1.02 (d, J = 6.8 Hz, 3H), 1.66 (br d, J = 14.0 Hz, 1H), 2.19 (septet, J = 6.7 Hz, 1H), 2.74 (m, 1H), 3.50 (brs, 1H), 3.78 (br d, J = 11.3 Hz, 1H), 3.91 (br dd, J = 11, 5 Hz, 1H), 3.97 (m, 1H), 4.22 (dd, J = 11.4, 1.7 Hz, 1H), 7.36 (br d, J = 7.6 Hz, 1H), 7.42 (ddd, J = 7.5, 7.5, 1.3 Hz, 1H), 7.48 (d, J = 8.6 Hz, 2H), 7.51 (m, 1H), 7.55 (br d, J = 7.5 Hz, 1H), 7.60 (d, J = 8.7 Hz, 2H). Retention time (HPLC)⁵ 4.40 min |
| 69 | 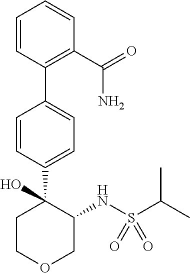 | 4′-{(3R,4S)-4- hydroxy-3- [(isopropylsulfonyl) amino] tetrahydro-2H- pyran-4- yl}biphenyl-2- carboxamide | LCMS m/z 419.7 (M + 1). ¹H NMR (400 MHz, METHANOL-d₄) δ 0.81 (d, J = 6.6 Hz, 3H), 1.02 (d, J = 6.8 Hz, 3H), 1.66 (br d, J = 14.0 Hz, 1H), 2.19 (septet, J = 6.8 Hz, 1H), 2.74 (m, 1H), 3.50 (brs, 1H), 3.78 (br d, J = 11.4 Hz, 1H), 3.91 (br dd, J = 11, 5 Hz, 1H), 3.97 (m, 1H), 4.22 (dd, J = 11.4, 1.7 Hz, 1H), 7.36 (br d, J = 7.7 Hz, 1H), 7.42 (ddd, J = 7.5, 7.5, 1.4 Hz, 1H), 7.48 (d, J = 8.6 Hz, 2H), 7.51 (m, 1H), 7.55 (br d, J = 7.5 Hz, 1H), 7.60 (d, J = 8.6 Hz, 2H). Retention time (HPLC)⁵ 6.78 min |
| 70 | 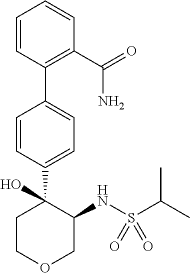 | 4′-{(3S,4S)-4- hydroxy-3- [(isopropylsulfonyl) amino] tetrahydro-2H- pyran-4- yl}biphenyl-2- carboxamide | LCMS m/z 419.7 (M + 1). ¹H NMR (400 MHz, METHANOL-d₄) δ 0.73 (d, J = 6.7 Hz, 3H), 1.01 (d, J = 6.8 Hz, 3H), 1.72 (br d, J = 14.2 Hz, 1H), 2.26 (septet, J = 6.7 Hz, 1H), 2.36 (m, 1H), 3.71 (m, 2H), 3.79 (br dd, J = 11, 5, 1H), 3.85-3.92 (m, 2H), 7.36 (br dd, J = 7.6, 1.4 Hz, 1H), 7.42 (ddd, J = 7.5, 7.5, 1.4 Hz, 1H), 7.49 (d, J = 8.6 Hz, 2H), 7.50 (m, 1H), 7.54 (br dd, J = 7.5, 1.6 Hz, 1H), 7.63 (d, J = 8.6 Hz, 2H). Retention time (HPLC)⁵ 6.00 min |
| 71 | 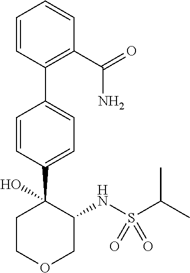 | 4′-{(3R,4R)-4- hydroxy-3- [(isopropylsulfonyl) amino] tetrahydro-2H- pyran-4- yl}biphenyl-2- carboxamide | LCMS m/z 419.7 (M + 1). ¹H NMR(400 MHz, METHANOL-d₄) δ 0.73 (d, J = 6.6 Hz, 3H), 1.01 (d, J = 6.8 Hz, 3H), 1.71 (br d, J = 14.4 Hz, 1H), 2.26 (septet, J = 6.6 Hz, 1H), 2.36 (m, 1H), 3.71 (m, 2H), 3.79 (br dd, J = 11, 5 Hz, 1H), 3.85-3.92 (m, 2H), 7.36 (br d, J = 7.7 Hz, 1H), 7.42 (br dd, J = 7.4, 7.4 Hz, 1H), 7.49 (d, J = 7.9 Hz, 2H), 7.48-7.55 (m, 2H), 7.63 (d, J = 8.0 Hz, 2H). Retention time (HPLC)⁵ 8.00 min |
| 72 | 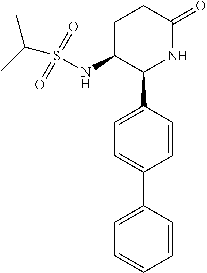 | N-[(2S,3S)-2- biphenyl-4-yl- 6- oxopiperidin- 3-yl]propane- 2-sulfonamide | LCMS m/z 371.1 (M − 1). ¹H NMR (500 MHz, CHLOROFORM-d) δ 1.13 (d, J = 6.8 Hz, 3 H), 1.24 (d, J = 6.8 Hz, 3 H), 1.99 (m, 1 H), 2.12 (m, 1 H), 2.63 (m, 1 H), 2.72 (m, 1 H), 2.93 (m, 1 H), 4.01 (m, 1H), 4.19 (d, J = 9.8 Hz, 1 H), 4.90 (br s, 1 H), 6.11 (br s, 1H), 7.39 (br t, J = 7.3 Hz, 1 H), 7.42 (d, J = 8.1 Hz, 2 H), 7.47 (dd, apparent t, J = 7.6 Hz, 2 H), 7.59 (d, J = 8.4 Hz, 2 H), 7.67 (d, J = 8.3 Hz, 2 H) |

¹HPLC conditions: Chiralpak AD-H column; 5 μm, 2.1 × 25 cm; Flow rate 65 g/min; Eluant: 70:30 CO₂:ethanol.
²HPLC conditions: Chiralpak AS-H column; 5 μm, 1.0 × 25 cm; Flow rate 10 g/min; Eluant: 70:30 CO₂:methanol.
³HPLC conditions: Chiralpak AS-H column; 5 μm, 1 × 25 cm; Flow rate 10 g/min; Eluant: 75:25 CO₂:ethanol.
⁴HPLC conditions: Chiralpak AS-H column; 5 μm, 1 × 25 cm; Flow rate 10 g/min; Eluant: 80:20 CO₂:methanol.
⁵HPLC conditions: Chiralpak AS-H column; 5 μm, 1 × 25 cm; Flow rate 10 g/min; Eluant: 65:35 CO₂:ethanol.

In the foregoing Table, Examples 14-23, 28-30, 35-41, 45-48, and 50-62 were performed using methods analogous to those of Example 1. Examples 24-27, 32-34, 42-44 and 49 were performed using methods analogous to Example 2. Example 31 was performed using methods analogous to Example 9, but using KF and THF rather than sodium carbonate and methanol/toluene/water.

Biological Protocols

Materials and Methods

Growth and Maintenance of ES Cells

The murine ES cell line used was E14-Sx1-16C, which has a targeted mutation in the Sox1 gene, a neuroectodermal marker, that offers G418 resistance when the Sox1 gene is expressed (Stem Cell Sciences). ES cells were maintained undifferentiated as previously described (Roach). Briefly, ES cells were grown in SCML media that had a base medium of Knockout™ D-MEM (Invitrogen), supplemented with 15% ES qualified Fetal Bovine Serum (FBS) (Invitrogen), 0.2 mM L-Glutamine (Invitrogen), 0.1 mM MEM non-essential amino acids (Invitrogen), 30 μg/ml Gentamicin (Invitrogen), 1000 u/ml ESGRO (Chemicon) and 0.1 mM 2-Mercaptoethanl (Sigma). ES cells were plated on gelatin-coated dishes (BD Biosciences), the media was changed daily and the cells were dissociated with 0.05% Trypsin EDTA (Invitrogen) every other day.

Neural In Vitro Differentiation of ES Cells

Embryoid Body Formation: Prior to embryoid body (EB) formation the ES cells were weaned from FBS onto Knockout Serum Replacement (KSR) (Invitrogen). To form EBs, ES cells were dissociated into a single cell suspension, then 3×106 cells were plated in bacteriology dishes (Nunc 4014) and grown as a suspension culture in NeuroEB-I medium that consisted of Knockout™ D-MEM (Invitrogen), supplemented with 10% KSR (Invitrogen), 0.2 mM L-Glutamine (Invitrogen), 0.1 mM MEM non-essential amino acids (Invitrogen), 30 μg/ml Gentamicin (Invitrogen), 1000 u/ml ESGRO (Chemicon), 0.1 mM 2-Mercaptoethanl (Sigma) and 150 ng/ml Transferrin (Invitrogen). The plates were put on a Stovall Belly Button shaker in an atmospheric oxygen incubator. The media was changed on day 2 of EB formation with NeuroEB-I and on day 4 with NeuroEB-II (NeuroEB-I plus 1 μg/ml mNoggin [R&D Systems]).

Neuronal Precursor Selection and Expansion: On day 5 of EB formation, EBs were dissociated with 0.05% Trypsin EDTA, and 4×10⁶cells/100 mm dish were plated on Laminin coated tissue culture dishes in Neuroll-G418 medium that consisted of a base medium of a 1:1 mixture of D-MEM/F12 supplemented with N2 supplements and NeuroBasal Medium supplemented with B27 supplement and 0.1 mM L-Glutamine (all from Invitrogen). The base medium was then supplemented with 10 ng/ml bFGF (Invitrogen), 1 μg/ml mNoggin, 500 ng/ml SHH—N, 100 ng/ml FGF-8b (R&D Systems), 1 μg/ml Laminin and 200 μg/ml G418 (Invitrogen) for selection of neuronal precursors expressing Sox-1. The plates were put in an incubator that contained 2% Oxygen and were maintained in these conditions. During the 6-day selection period, the Neuroll media was changed daily. On day 6, the surviving neuronal precursor foci were dissociated with 0.05% Trypsin EDTA and the cells were plated at a density of 1.5×10⁶ cells/100 mm Laminin coated dish in Neuroll-G418 medium. The cells were dissociated every other day for expansion, and prepared for Cryopreservation at passage 3 or 4. The crypreservation medium contained 50% KSR, 10% Dimethyl Sulfoxide (DMSO) (Sigma) and 40% Neurol-G418I medium. Neuronal precursors were crypreserved at a concentration of 4×10⁶ cells/ml and 1 ml/cryovial in a controlled rate freezer overnight then transferred to an ultra-low freezer or liquid nitrogen for long-term storage.

Neuronal Differentiation: Cryopreserved ES cell-derived neuronal precursors were thawed by the rapid thaw method in a 37-degree water-bath. The cells were transferred from the cryovial to a 100 mm Laminin coated tissue culture dish that already contained Neuroll-G418 that had been equilibrated in a 2% Oxygen incubator. The media was changed with fresh Neuroll-G418 the next day. The cells were dissociated every other day as described above for expansion to generate enough cells to plate for the screen. For the screen, the cells were plated into 384-well poly-d-lysine coated tissue culture dishes (BD Biosciences) by the automated SelecT at a cell density of 6K cells/well in differentiation medium Neurolll that contained a 4:1 ratio of the NeuroBasalMedium/B27:D-MEM/F12/N2 supplemented with 1 μM cAMP (Sigma), 200 μM Ascorbic Acid (Sigma), 1 μg/ml Laminin (Invitrogen) and 10 ng/ml BDNF (R&D Systems). The plates were put in an incubator with 2% Oxygen and allowed to complete the differentiation process for 7 days. The cells could then be used over a 5-day period for the high throughput screen.

In Vitro Assays

Procedure for AMPA ES Cell FLIPR Screen

FLIPR Methods and Data Analysis:

On the day of the assay, the FLIPR assay may be performed using the following methods:

Assay buffer:

| Compound | g/L | MW | [concentration] |
|---|---|---|---|
| NaCl | 8.47 | 58.44 | 145 mM |
| Glucose | 1.8 | 180.2 | 10 mM |
| KCl | .37 | 74.56 | 5 mM |
| MgSO₄ | 1 ml 1M Stock | 246.48 | 1 mM |
| HEPES | 2.38 | 238.3 | 10 mM |
| CaCl₂ | 2 ml 1M Stock | 110.99 | 2 mM |

The pH is adjusted to 7.4 with 1M NaOH. Prepare a 2 mM (approx.) stock solution of Fluo-4,am (Invitrogen) dye in DMSO-22 μl DMSO per 50 μg vial (440 μL per 1 mg vial). Make a 1 mM (approx.) flou-4, PA working solution per vial by adding 22 μl of 20% pluronic acid (PA) (Invitrogen) in DMSO to each 50 μg vial (440 μL per 1 mg vial). Prepare a 250 mM Probenecid (Sigma) stock solution. Make 4 μM (approx.) dye incubation media by adding the contents of 2 50 μg vials per 11 ml DMEM high glucose without glutamine (220 ml DMEM per 1 mg vial). Add 110 μL probenecid stock per 11 ml media (2.5 mM final concentration). Dye concentrations ranging from 2 μM to 8 μM dye can be used without altering agonist or potentiator pharmacology. Add probenecid to the assay buffer used for cell washing (but not drug preparation) at 110 μl probenecid stock per 11 ml buffer.

Remove growth media from cell plates by flicking. Add 50 μl/well dye solution. Incubate 1 hour at 37° C. and 5% CO₂. Remove dye solution and wash 3 times with assay buffer+probenecid (100 μl probenecid stock per 10 ml buffer), leaving 30 μL/well assay buffer. Wait at least 10-15 minutes. Compound and agonist challenge additions are performed with the FLIPR (Molecular Devices). The 1^{st} addition is for test compounds, which are added as 15 μL of a 4× concentration. The second 2^{nd} addition is 15 μL of 4× concentration of agonist or challenge. This achieves 1× concentration of all compounds only after 2^{nd} addition. Compounds are pretreated at least 5 minutes before agonist addition.

Several baseline images are collected with the FLIPR before compound addition, and images are collected for least one minute after compound addition. Results are analyzed by subtracting the minimum fluorescent FLIPR value after compound or agonist addition from the peak fluorescent value of the FLIPR response after agonist addition to obtain the change in fluorescence. The change in fluorescence (RFUs, relative fluorescent units) are then analyzed using standard curve fitting algorithms. The negative control is defined by the AMPA challenge alone, and the positive control is defined by the AMPA challenge plus a maximal concentration of cyclothiazide (10 uM or 32 uM).

Compounds are delivered as DMSO stocks or as powders. Powders are solubilized in DMSO. Compounds are then added to assay drug buffer as 40 μL top [concentration] (4× the top screening concentration). The standard agonist challenge for this assay is 32 uM AMPA.

EC₅₀ values of the compounds of the invention are preferably 10 micromolar or less, more preferably 1 micromolar or less, even more preferably 100 nanomolar or less. The data for specific compounds of the invention is provided below in Table 1.

TABLE 1

| Example | AMPA Potentiator | |
|---|---|---|
| Number | Assay EC₅₀ | |
| 1 | 11.0 | uM |
| 2 | 0.365 | uM |
| 3 | 4.30 | uM |
| 4 | >31.6 | uM |
| 5 | 7.66 | uM |
| 6 | >31.6 | uM |
| 7 | >31.6 | uM |
| 8 | 0.403 | uM |
| 9 | 4.45 | uM* |
| 10 | >7.52 | uM* |
| 11 | >31.6 | uM |
| 12 | 12.2 | uM* |
| 13 | >31.6 | uM |
| 14 | | |
| 15 | >31.6 | uM |
| 16 | 1.84 | uM |
| 17 | >31.6 | uM |
| 18 | 0.415 | uM |
| 19 | >31.6 | uM |
| 20 | 9.79 | uM |
| 21 | >31.6 | uM* |
| 22 | 0.429 | uM* |
| 23 | 10.3 | uM |
| 24 | 0.378 | uM |
| 25 | 0.684 | uM |
| 26 | >31.6 | uM |
| 27 | 0.320 | uM |
| 28 | >31.6 | uM |
| 29 | 4.04 | uM* |
| 30 | >31.6 | uM* |
| 31 | 0.318 | uM* |
| 32 | >26.6 | uM* |
| 33 | >31.6 | uM |
| 34 | 0.220 | uM* |
| 35 | 4.57 | uM |
| 36 | >31.6 | uM |
| 37 | >4.67 | uM* |
| 38 | 1.04 | uM |
| 39 | >31.6 | uM |
| 40 | 0.255 | uM |
| 41 | 0.826 | uM* |
| 42 | >31.6 | uM |
| 43 | >31.6 | uM |
| 44 | 0.493 | uM* |
| 45 | >9.61 | uM* |
| 46 | 1.24 | uM* |
| 47 | 20.3 | uM |
| 48 | 5.64 | uM* |
| 49 | >31.6 | uM |
| 50 | 1.71 | uM* |
| 51 | 1.71 | uM* |
| 52 | >31.6 | uM |
| 53 | 2.36 | uM |
| 54 | 2.29 | uM |
| 55 | >31.6 | uM |
| 56 | >31.6 | uM |
| 57 | 0.748 | uM |
| 58 | >31.6 | uM |
| 59 | 2.10 | uM |
| 60 | >31.6 | uM |
| 61 | >31.6 | uM |
| 62 | >31.6 | uM |
| 63 | 0.669 | uM |

*Value represents the geometric mean of 2-9 EC₅₀ determinations

When introducing elements of the present invention or the exemplary embodiment(s) thereof, the articles “a,” “an,” “the” and “said” are intended to mean that there are one or more of the elements. The terms “comprising,” “including” and “having” are intended to be inclusive and mean that there may be additional elements other than the listed elements. Although this invention has been described with respect to specific embodiments, the details of these embodiments are not to be construed as limitations to the invention, the scope of which is defined by the appended claims.

1. A compound of Formula I, or a pharmaceutically acceptable salt thereof, 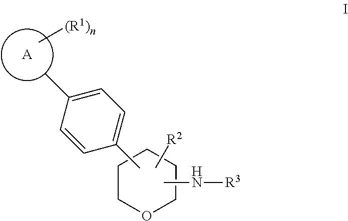
wherein R¹ is hydrogen, fluoro, (C₁-C₂)alkyl optionally substituted with one to five fluoro, (C₁-C₂)alkoxy optionally substituted with one to five fluoro, —(C═O)—NH₂, —(C═O)—NH—(C₁-C₂)alkyl, —(C═O)—N[C₁-C₂)alkyl]₂ or —CN;
n is an integer from zero, one, or two;
R² is hydrogen or hydroxyl;
R³ is (C₁-C₅)alkyl-(C═O)—, [(C₁-C₂)alkyl]₂N—(C═O)—, (C₁-C₅)alkyl-SO₂—, (C₃-C₅)cycloalkyl-SO₂—, [(C₁-C₂)alkyl]₂N—SO₂—; wherein said (C₁-C₂)alkyl moieties of said [(C₁-C₂)alkyl]₂N—(C═O)— and [(C₁-C₂)alkyl]₂N—SO₂— may optionally be taken together with the Nitrogen atom to which they are attached to form a three to six membered heterocyclic ring;
and ring “A” is phenyl, pyridyl, or thienyl.

2. A compound of Formula I, according to claim 1, with the stereochemistry depicted below for formula Ia:

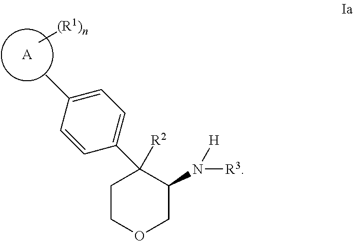

3. A compound of Formula I, according to claim 1, with the stereochemistry depicted below for formula Ib:

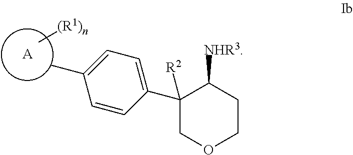

4. A compound of Formula I according to claim 1, with the stereochemistry about the pyranyl ring as depicted below for formula Ic:

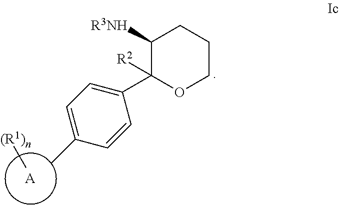

5. A compound of Formula I, according to claim 1, with the stereochemistry about the pyranyl ring as depicted below for formula Id:

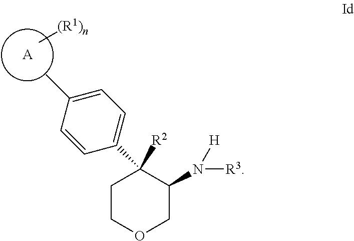

6. A compound of Formula I according to claim 1, with the stereochemistry about the pyranyl ring as depicted below for formula Ie:

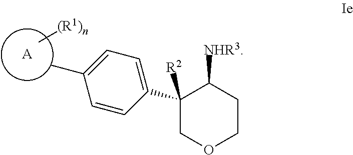

7. A compound of Formula I according to claim 1, with the stereochemistry about the pyranyl ring as depicted below for formula If:

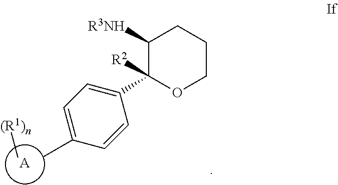

8. A compound of Formula I, according to claim 1, with the stereochemistry about the pyranyl ring as depicted below for Formula Ig:

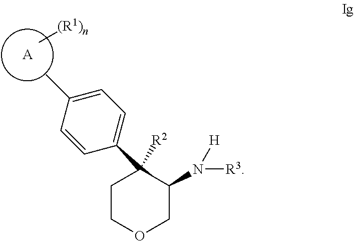

9. A compound of Formula I, according to claim 1, with the stereochemistry about the pyranyl ring as depicted below for Formula Ih:

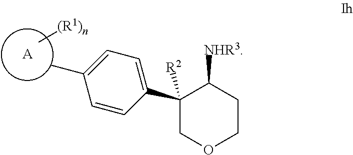

10. A compound of Formula I, according to claim 1, with the stereochemistry about the pyranyl ring as depicted below for Formula Ii:

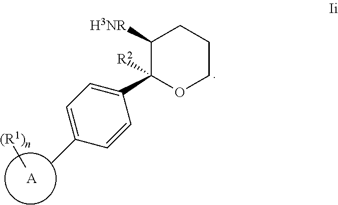

11. A compound according to claim 1, or a pharmaceutically acceptable salt thereof, wherein said compound is:
N-[(3R,4S)-3-biphenyl-4-yltetrahydro-2H-pyran-4-yl]propane-2-sulfonamide;
N-[(3R,4R)-4-(2′-ethoxybiphenyl-4-yl)tetrahydro-2H-pyran-3-yl]propane-2-sulfonamide;
N-[(3R,4R)-4-biphenyl-4-yltetrahydro-2H-pyran-3-yl]propane-2-sulfonamide;
N-[(3R,4R)-4-(2′-cyano biphenyl-4-yl)tetrahydro-2H-pyran-3-yl]propane-2-sulfonamide;
N-[(3S,4S)-3-(2′-cyano biphenyl-4-yl)tetrahydro-2H-pyran-4-yl]propane-2-sulfonamide;
N-[(3S,4S)-3-(2′-cyano-4′-fluorobiphenyl-4-yl)tetrahydro-2H-pyran-4-yl] propane-2-sulfonamide;
N-[(3S,4S)-3-biphenyl-4-yltetrahydro-2H-pyran-4-yl]propane-2-sulfonamide;
N-[(3S,4S)-3-(2′-ethoxybiphenyl-4-yl)tetrahydro-2H-pyran-4-yl]propane-2-sulfonamide;
N-[(3R,4S)-3-(2′-cyanobiphenyl-4-yl)tetrahydro-2H-pyran-4-yl]propane-2-sulfonamide;
N-[(3R,4S)-3-(2′-ethoxybiphenyl-4-yl)tetrahydro-2H-pyran-4-yl]propane-2-sulfonamide;
N-[(3R,4S)-3-(4′-cyanobiphenyl-4-yl)tetrahydro-2H-pyran-4-yl]propane-2-sulfonamide;
N-[(3R,4S)-3-(2′-cyano-4′-fluorobiphenyl-4-yl)tetrahydro-2H-pyran-4-yl]propane-2-sulfonamide;
N-{(3R,4S)-3-[4-(5-cyano-2-thienyl)phenyl]tetrahydro-2H-pyran-4-yl}propane-2-sulfonamide;
N-[(2R,3S)-2-(2′-ethoxybiphenyl-4-yl)tetrahydro-2H-pyran-3-yl]propane-2-sulfonamide;
N-[(2R,3S)-2-(2′-cyanobiphenyl-4-yl)tetrahydro-2H-pyran-3-yl]propane-2-sulfonamide;
N-[(3R,4S)-4-(2′-cyanobiphenyl-4-yl)tetrahydro-2H-pyran-3-yl]propane-2-sulfonamide; or
N-[(3R,4S)-4-(2′-ethoxybiphenyl-4-yl)tetrahydro-2H-pyran-3-yl]propane-2-sulfonamide.

12. A compound according to claim 1, or a pharmaceutically acceptable salt thereof, wherein said compound is:
N-[(3R,4R)-4-(2′-cyanobiphenyl-4-yl)tetrahydro-2H-pyran-3-yl]propane-2-sulfonamide;
N-{(3R,4R)-4-[4-(5-cyano-2-thienyl)phenyl]tetrahydro-2H-pyran-3-yl}propane-2-sulfonamide;
N-[(3R,4R)-3-(2′-cyanobiphenyl-4-yl)tetrahydro-2H-pyran-4-yl]propane-2-sulfonamide;
N-{(3S,4S)-3-[4-(5-cyano-2-thienyl)phenyl]tetrahydro-2H-pyran-4-yl}propane-2-sulfonamide;
N-[(3S,4S)-3-(2′-cyanobiphenyl-4-yl)tetrahydro-2H-pyran-4-yl]propane-2-sulfonamide;
N-[(3S,4S)-3-(2′-cyano-4′-fluorobiphenyl-4-yl)tetrahydro-2H-pyran-4-yl]propane-2-sulfonamide;
N-[(3S,4S)-3-(4′-cyanobiphenyl-4-yl)tetrahydro-2H-pyran-4-yl]propane-2-sulfonamide;
N-[(3R,4S)-3-(2′-cyanobiphenyl-4-yl)tetrahydro-2H-pyran-4-yl]propane-2-sulfonamide;
N-{(3R,4S)-4-[4-(5-cyano-2-thienyl)phenyl]tetrahydro-2H-pyran-3-yl}propane-2-sulfonamide;
N-[(3S,4R)-3-(2′-ethoxybiphenyl-4-yl)tetrahydro-2H-pyran-4-yl]propane-2-sulfonamide;
N-[(3S,4R)-3-(4-pyridin-3-ylphenyl)tetrahydro-2H-pyran-4-yl]propane-2-sulfonamide;
N-[(2S,3R)-2-(2′-ethoxybiphenyl-4-yl)tetrahydro-2H-pyran-3-yl]propane-2-sulfonamide;
N-[(2S,3R)-2-(2′-cyanobiphenyl-4-yl)tetrahydro-2H-pyran-3-yl]propane-2-sulfonamide;
N-[(2R,3S)-2-biphenyl-4-yltetrahydro-2H-pyran-3-yl]propane-2-sulfonamide;
N-{(2R,3S)-2-[4-(5-cyano-2-thienyl)phenyl]tetrahydro-2H-pyran-3-yl}propane-2-sulfonamide;
N-[(3S,4R)-4-biphenyl-4-yl-4-hydroxytetrahydro-2H-pyran-3-yl]propane-2-sulfonamide; or
N-[(3R,4S)-4-biphenyl-4-yl-4-hydroxytetrahydro-2H-pyran-3-yl]propane-2-sulfonamide.

13. A compound of Formula II, or a pharmaceutically acceptable salt thereof, 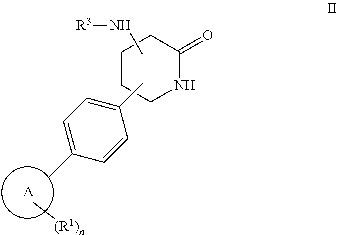
wherein R¹ is hydrogen, fluoro, (C₁-C₂)alkyl optionally substituted with one to five fluoro, (C₁-C₂)alkoxy optionally substituted with one to five fluoro, —(C═O)—NH₂, —(C═O)—NH—(C₁-C₂)alkyl, —(C═O)—N[(C₁-C₂)alkyl]₂ or —CN;
n is an integer from zero, one, or two;
R³ is (C₁-C₅)alkyl-(C═O)—, [(C₁-C₂)alkyl]₂N—(C═O)—, (C₁-C₅)alkyl-SO₂—, (C₃-C₅)cycloalkyl-SO₂—, or [(C₁-C₂)alkyl]₂N—SO₂—; wherein said (C₁-C₂)alkyl moieties of said [(C₁-C₂)alkyl]₂N—(C═O)— and [(C₁-C₂)alkyl]₂N—SO₂— may optionally be taken together with the nitrogen atom to which they are attached to form a three to six membered heterocyclic ring;
and ring “A” is phenyl, pyridyl, or thienyl.

14. A compound of Formula II according to claim 13, with the stereochemistry about the piperidone ring as depicted below for Formula IIa:

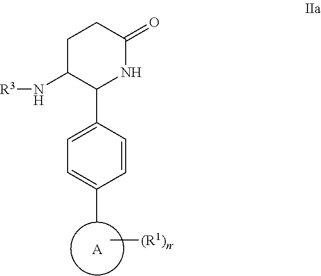

15. A compound of Formula II according to claim 13, with the stereochemistry about the piperidone ring as depicted below for Formula IIb:

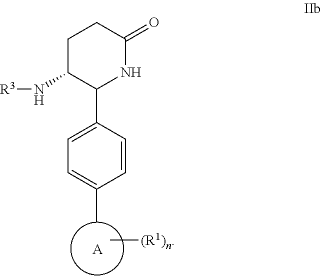

16. A compound of Formula II according to claim 13, with the stereochemistry about the piperidone ring as depicted below for Formula IIc:

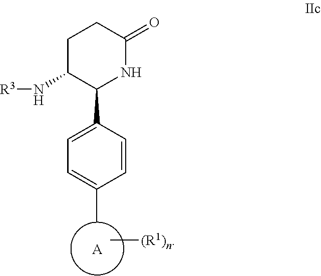

17. The compound according to claim 13, or a pharmaceutically acceptable salt thereof, wherein said compound is
N-[(2S,3R)-2-(2′-ethoxybiphenyl-4-yl)-6-oxopiperidin-3-yl]propane-2-sulfonamide;
N-[(2R,3S)-2-(2′-ethoxybiphenyl-4-yl)-6-oxopiperidin-3-yl]propane-2-sulfonamide;
N-[(2S,3R)-2-biphenyl-4-yl-6-oxopiperidin-3-yl]propane-2-sulfonamide;
N-{(2S,3R)-2-[4-(5-cyano-2-thienyl)phenyl]-6-oxopiperidin-3-yl}propane-2-sulfonamide;
N-{(2R,3S)-2-[4-(5-cyano-2-thienyl)phenyl]-6-oxopiperidin-3-yl}propane-2-sulfonamide;
or
N-[(2R,3S)-2-(2′-cyanobiphenyl-4-yl)-6-oxopiperidin-3-yl]propane-2-sulfonamide, or the pharmaceutically acceptable salts thereof.

18. A compound according to claim 1 or a pharmaceutically acceptable salt thereof, wherein R¹ is cyano, ethoxy or fluoro and is in the ortho or para position.

19. A method for the treatment or prevention in a mammal of a condition selected from the group consisting of acute neurological and psychiatric disorders, stroke, cerebral ischemia, spinal cord trauma, head trauma, perinatal hypoxia, cardiac arrest, hypoglycemic neuronal damage, dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine, urinary incontinence, substance tolerance, substance withdrawal, psychosis, schizophrenia, anxiety, mood disorders, trigeminal neuralgia, hearing loss, tinnitus, macular degeneration of the eye, emesis, brain edema, pain, tardive dyskinesia, sleep disorders, attention deficit/hyperactivity disorder, attention deficit disorder, and conduct disorder, comprising administering a compound of claim 1, or a pharmaceutically acceptable salt thereof, to the mammal.

20. A pharmaceutical composition comprising a compound according to claim 1 or a pharmaceutically acceptable salt thereof, and a pharmaceutically acceptable carrier.
